(19) United States
(12) Patent Application Publication
(10) Pub. No.: US 2013/0095039 A1
(43) Pub. Date: Apr. 18, 2013

(54) NUCLEIC ACID-MEDIATED SHAPE CONTROL OF NANOPARTICLES

(71) Applicant: The Board of Trustees of the University of Illinois, Urbana, IL (US)

(72) Inventors: Yi Lu, Champaign, IL (US); Zidong Wang, Urbana, IL (US)

(73) Assignee: The Board of Trustees of the University of Illinois, Urbana, IL (US)

(21) Appl. No.: 13/717,535

(22) Filed: Dec. 17, 2012

Related U.S. Application Data
(63) Continuation-in-part of application No. 13/249,070, filed on Sep. 29, 2011.
(60) Provisional application No. 61/404,410, filed on Sep. 30, 2010.
(60) Provisional application No. 61/576,867, filed on Dec. 16, 2011.

(51) Int. Cl. A61K 49/00

(52) U.S. Cl. CPC A61K 49/0002; Y10S 977/81; Y10S 977/904; Y10S 977/906; B82Y 5/00
USPC 424/9.1; 435/375; 428/403; 977/810; 977/904; 977/906

(57) ABSTRACT

Embodiments of a method to use nucleic acid oligomer sequences for modulating the shape of nanoparticles are disclosed, as well as nanoparticles and methods of using the nanoparticles. Systematic variations of the nucleic acid sequences offer mechanistic insights into the morphology control. A plurality of nucleic acid oligomers is adsorbed onto a metal nanoseed to provide an oligomer-functionalized nanoparticle. Additional metal is deposited onto the oligomer-functionalized nanoparticle to produce a shaped nanoparticle having a morphology based at least in part on the nanoseed morphology and the oligomer's sequence composition. Embodiments of methods for using the shaped nanoparticles also are disclosed.

### CROSS REFERENCE TO RELATED APPLICATIONS

This application is a continuation-in-part of U.S. application Ser. No. 13/249,070, filed Sep. 29, 2011, which claims the benefit of the earlier filing date of U.S. Provisional Patent Application No. 61/404,410, filed Sep. 30, 2010, and also claims the benefit of the earlier filing date of U.S. Provisional Application No. 61/576,867, filed Dec. 16, 2011, all of which are incorporated herein by reference in their entirety.

### ACKNOWLEDGMENT OF GOVERNMENT SUPPORT

This invention was made with government support under Grant Nos. CMMI0749028, CTS0120978, and DMR0117792 awarded by the National Science Foundation, and DEFG02-91-ER45439, awarded by the U.S. Department of Energy. The government has certain rights in the invention.

### FIELD

Embodiments of a method for using nucleic acid molecules to control the growth and shape of nanoparticles are disclosed, as well as nanoparticles and methods of using such nanoparticles.

### BACKGROUND

Metal nanoparticles have unique physicochemical properties leading to potential applications in selective catalysis, sensitive sensing, enhanced imaging, and medical treatment.^{1-9,53,54} The properties of a metal nanoparticle typically are affected by its size, shape, and crystal structure, and therefore it is possible to tune the properties of the particle by controlling its growth process. Molecular capping agents such as organic surfactants and polymers have been used to direct nanocrystal growth in a face-selective fashion to produce shape-controlled nanoparticle synthesis.^{8,9} Despite tremendous progress made, the mechanism of the shape control is not well understood, in part due to the difficulty in defining structures and conformations of these surfactants and polymers in solution and in systematic variation of functional groups.

DNA is a biopolymer with more defined structure and conformation in solution and unique programmable nature to tune its functional properties.¹⁰⁻¹³ Because of these advantages, DNA has been used as a template to position nanoparticles through DNA metallization,^{14,15} or nanoparticle attachment,¹⁶⁻²¹ or to control the sizes and/or the photo-luminescent properties of quantum dots.²²⁻²⁸ However, in contrast to proteins or peptides,²⁹⁻³² DNA has been much less explored to control the shape or morphology of metal nanoparticles, and, therefore the promise of this field remains to be fully realized. Such an investigation may result in new nanoparticles with new shapes and offer deeper insights into mechanisms of shape control.

### SUMMARY

Embodiments of a method to use nucleic acid oligomer sequences (such as DNA, PNA, or RNA) for modulating the shape of nanoparticles are disclosed, as well as shaped nanoparticles made by embodiments of the method and methods of using the shaped nanoparticles. The disclosure provides a novel “genetic code” for designing nanoparticles having a particular morphology. Systematic variations of the nucleic acid sequences offer mechanistic insights into the morphology control.

One embodiment of a method for making a shaped nanoparticle includes providing a metal nanoseed, selecting a nucleic acid oligomer having a sequence composition comprising at least two unique sequence segments, each sequence segment comprising nucleobases selected from the group consisting of A, C, G, T, U, and modified nucleobases, adsorbing a plurality of the nucleic acid oligomers onto the metal nanoseed to produce an oligomer-functionalized nanoseed, and depositing metal onto the oligomer-functionalized nanoseed to make a shaped nanoparticle, wherein the shaped nanoparticle has a morphology based at least in part on the oligomer sequence composition. In some embodiments, the nanoparticle's morphology—i.e. shape, surface characteristics, or a combination thereof—is subsequently determined. In some embodiments, the nanoparticle is subsequently detected (for example using microscopy, such as SEM or TEM).

In one embodiment, the method is repeated using a subsequent metal nanoseed having a substantially similar morphology to the first nanoseed and a subsequent nucleic acid oligomer comprising at least two subsequent sequence segments, each subsequent sequence segment comprising nucleobases selected from the group consisting of A, C, G, T, U, and modified nucleobases, wherein the subsequent oligomer sequence has a sequence variation in length of at least one sequence segment, type(s) of nucleobases in at least one sequence segment, or both, compared to the oligomer sequence. A shape effect and/or a surface characteristic effect of the sequence variation can then be determined based at least in part on the shaped nanoparticle's morphology and the subsequent shaped nanoparticle's morphology. Additionally, based at least in part on the oligomer sequence composition, the subsequent oligomer sequence composition, the shaped nanoparticle's morphology, and the subsequent shaped nanoparticle's morphology, a shape effect rule, a surface characteristic rule, or both may be determined. The rule may further be based at least in part on the nanoseed's morphology.

In certain embodiments, each sequence segment has a length of at least five nucleobases. In one embodiment, a nucleic acid oligomer having a sequence composition with a poly A sequence segment, a poly C sequence segment, or a combination thereof produces a shaped nanoparticle with surface roughness. In another embodiment, a nucleic acid oligomer having a sequence composition with a poly T sequence segment produces a shaped nanoparticle with a flattened surface, thickened edges relative to a center thickness, or a combination thereof. In yet another embodiment, a nucleic acid oligomer having a sequence composition with a poly G sequence segment produces a shaped nanoparticle with hexagonal characteristics.

In one embodiment, the nanoseed is a nanoprism and the nucleic acid oligomer includes a poly G segment and a poly T segment, thereby producing a hexagon-shaped nanoparticle when the nucleic acid oligomer has a sequence composition that is at least 50% poly G or a six-pointed bowl-shaped nanoparticle when the nucleic acid oligomer has a sequence composition that is less than 50% poly G, and the shaped nanoparticle has an edge thickness that is greater than a center thickness of the shaped nanoparticle. The edge thickness of the nanoparticle edges increases relative to the center thickness as the percent poly T in the nucleic acid oligomer increases.

In another embodiment, the nanoseed is a nanoprism, and the nucleic acid oligomer has a poly T segment and a poly C segment, thereby producing a flower-shaped nanoparticle comprising a plurality of edge tips having an average length and an average thickness. The average length and the average thickness of the edge tips increases as the percent poly T in the nucleic acid oligomer increases.

In another embodiment, the nanoseed is a nanoprism, and the nucleic acid oligomer includes a poly G sequence segment that is at least 50% of the nucleic acid oligomer, thereby producing a hexagon-shaped nanoparticle. If the nucleic acid oligomer further includes a poly T segment, a hexagon-shaped nanoparticle with a smooth surface morphology and an edge thickness that is greater than a center thickness of the nanoparticle is produced. The nanoparticle's edge thickness increases relative to its center thickness as the percent poly T in the nucleic acid oligomer increases. If the nucleic acid oligomer instead includes a poly A segment, a poly C segment, or a combination thereof, a hexagon-shaped nanoparticle with a rough surface morphology is produced. Further including a poly T segment produces an edge thickness that is greater than a center thickness of the nanoparticle, and the nanoparticle's edge thickness increases relative to the center thickness as the percent poly T in the nucleic acid oligomer increases.

In another embodiment, the nanoseed is a nanoprism, and the nucleic acid oligomer includes a poly A segment and a poly T segment, or a poly A segment and a poly C segment, thereby producing a round, plate-shaped nanoparticle with a rough surface morphology.

In one embodiment, a method for making a shaped nanoparticle having a desired morphology further includes first selecting a desired nanoparticle morphology, and selecting a nucleic acid oligomer having a sequence composition based at least in part on a morphology of the metal nanoseed and the desired nanoparticle morphology. Desired morphology may include a desired nanoparticle shape, desired nanoparticle surface characteristics, or a combination thereof.

In one embodiment, the desired nanoparticle morphology includes surface roughness, and selecting the oligomer sequence composition includes selecting a sequence composition including at least one sequence segment of poly A, poly C, or a combination of A and C. In another embodiment, the desired nanoparticle morphology includes surface flattening, thickened edges relative to a center thickness of the shaped nanoparticle, or a combination thereof, and selecting the oligomer sequence composition comprises selecting a sequence composition including at least one sequence segment of poly T. In one embodiment, increasing the percent poly T in the nucleic acid oligomer increases an edge thickness of the shaped nanoparticle relative to the center thickness of the shaped nanoparticle.

In one embodiment, the desired nanoparticle morphology includes a hexagonal shape, and selecting the nucleic acid oligomer includes selecting a nucleic acid oligomer having a sequence composition with at least one sequence segment of poly G, wherein the nucleic acid oligomer is at least 50% poly G. In another embodiment, the desired nanoparticle morphology further includes a flattened surface, thickened edges relative to a center thickness of the shaped nanoparticle, or a combination thereof, and selecting the oligomer sequence composition further comprises selecting a sequence composition including at least one sequence segment comprising poly T, and wherein increasing the percent poly T in the nucleic acid oligomer increases the edge thickness relative to the center thickness. In another embodiment, the desired nanoparticle morphology further includes a rough surface, and selecting the oligomer sequence composition further comprises selecting a sequence composition including at least one sequence segment of poly A, poly C, or a combination thereof. If the desired nanoparticle morphology further includes an edge thickness that is greater than a center thickness, then selecting the nucleic acid oligomer includes selecting a sequence composition including at least one sequence segment comprising poly T, wherein increasing the percent poly T in the nucleic acid oligomer increases the edge thickness relative to the center thickness.

In one embodiment, the desired nanoparticle morphology is a six-pointed bowl shape, and selecting the oligomer sequence composition comprises selecting a sequence composition including at least one sequence segment of poly T and at least one sequence segment of poly G, wherein the nucleic acid oligomer is at least 50% poly T, wherein increasing the percent poly T in the nucleic acid oligomer increases an edge thickness of the shaped nanoparticle relative to a center thickness of the shaped nanoparticle.

In one embodiment, the desired nanoparticle morphology is a flower shape with multiple edge tips having an average length and an average thickness, and selecting the oligomer sequence composition comprises selecting a sequence composition including at least one sequence segment comprising poly T and at least one sequence segment comprising poly C, wherein increasing the percent poly T in the nucleic acid oligomer increases the average length and the average thickness of the edge tips.

Methods are provided for using the disclosed shaped nanoparticles made by the methods provided herein, for example for delivery of a desired agent to a target cell. Exemplary agents include antibodies, therapeutic drugs, aptamers, proteins, peptides, nucleic acids (such as DNA or RNA, for example mRNA, inhibitory RNAs such as siRNAs, genes, and gene fragments). Thus provided are methods for delivering a shaped nanoparticle to a target cell, wherein the method can include providing a shaped nanoparticle-agent (e.g., antibody) conjugate, wherein the antibody is specific for a protein on the surface of a target cell; and contacting the shaped nanoparticle with the target cell under conditions that allow the shaped nanoparticle to bind to and/or enter the cell, thereby delivering the shaped nanoparticle to the target cell.

In another example, the desired agent is a drug, and the method can include providing a shaped nanoparticle-drug conjugate and contacting the shaped nanoparticle-drug conjugate with the cell, wherein the shaped nanoparticle-drug conjugate is contacted with the cell under conditions sufficient to allow the cell to internalize the shaped nanoparticle-drug conjugate.

In another example, a subject is administered a shaped nanoparticle comprising a metal nanoparticle, and a plurality of nucleic acid oligomers, each nucleic acid oligomer having an oligomer sequence composition comprising at least two unique sequence segments, each sequence segment having a length of at least five nucleobases selected from the group consisting of A, C, G, T, U, and modified bases, and the shaped nanoparticle is detected in the subject (e.g., by CT or x-ray imaging).

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1a depicts UV-visible spectra of gold nanoparticle solutions prepared with A30 oligomers (SEQ ID NO: 4) (AuNF_A30, dark blue line), C30 oligomers (SEQ ID NO: 6) (AuNF_C30, blue line), T30 oligomers (SEQ ID NO: 5) (AuNF_T30, red line), in the absence of DNA (AuNF_No DNA, pink line), or before reduction (AuNS/No reduction, light pink line); FIG. 1b is a series of color images of the corresponding gold nanoparticles.

FIGS. 2a-2d are a series of transmission electron microscopy (TEM) images of gold nanoparticles prepared from gold nanospheres with (2a) A30 oligomers (SEQ ID NO: 4), (2b) C30 oligomers (SEQ ID NO: 6), (2c) T30 oligomers (SEQ ID NO: 5), (2d) in the absence of DNA. The scale bar indicates 20 nm.

FIG. 3 is a TEM image of gold nanoparticles prepared with G10 (SEQ ID NO: 18) oligomers. The scale bar indicates 20 nm.

FIG. 4a is a TEM image of 200-nm gold nanoseeds (AuNS).

FIG. 4b is a TEM image of gold nanoparticles prepared in the absence of DNA but with the addition of 20 mM NaCl. It is noted that aggregation of the gold nanoparticles occurred during synthesis.

FIGS. 5a-5d are color images of AuNS solutions incubated with (a) A30 oligomers (SEQ ID NO: 4), (b) C30 oligomers (SEQ ID NO: 6), (c) T30 oligomers (SEQ ID NO: 5), and (d) in the absence of DNA before (left image of each pair) and after (right image of each pair) the addition of 0.1 M NaCl.

FIG. 5e is a series of UV-visible spectra of the corresponding nanoparticle solutions with and without the presence of 0.1 M NaCl.

FIGS. 6a-6f are TEM images of gold nanoparticles prepared by reducing (a) 0.05 μL, (b) 0.1 μL, (c) 0.4 μL, (d) 0.6 μL, (e) 1.2 μL, and (f) 2.0 μL of 1% HAuCl₄ aqueous solution with an excess amount of NH₂OH (20 mM). Before the reduction reaction, 100 μL of 0.5 nM AuNS solution was incubated with 1 μM poly A30 (SEQ ID NO: 4). The scale bar indicates 20 nm.

FIGS. 7a-7f are TEM images of gold nanoparticles prepared by incubating AuNS solutions with poly A30 (SEQ ID NO: 4) at different molar ratios: AuNS:DNA=(a) 1:20, (b) 1:100, (c) 1:500, (d) 1:1000, (e) 1:2000, (f) 1:4000. The AuNS solutions (0.5 nM) were incubated with DNA for 30 minutes, followed by addition of 20 mM NH₂OH and 167 μM HAuCl₄ to complete the nanoparticle synthesis. The scale bar indicates 20 nm.

FIGS. 8a-8b are TEM images of gold nanoparticles prepared with (a) adenosine monophosphate (AMP), and (b) random 30-mer DNA. A similar synthesis procedure was followed except that 0.5 nM AuNS was incubated with 30 μM AMP or 1 μM random DNA with the sequence 5′-AGT CAC GTA TAC AGC TCA TGA TCA GTC AGT-3′ (SEQ ID NO: 3). The scale bar indicates 20 nm.

FIG. 9 depicts the time-dependent evolution of the UV-visible spectra of gold nanoflowers (AuNF) grown in the presence of A30 oligomers (SEQ ID NO: 4). From bottom to top, the spectra illustrate the absorbance of the growth solution after initiation of the reaction for 0 s, 3 s, 5 s, 10 s, 30 s, 60 s, 120 s, 240 s, 480 s, 720 s, and 840 s, respectively.

FIGS. 10a-10r are TEM images of the nanoparticle intermediates prepared by stopping the nanoparticle growth with mercaptopropionic acid (1.5 mM) after 0.5 s (a, g, m), 2 s (b, h, n), 5 s (c, i, o), 30 s (d, j, p), 5 min. (e, k, q) and 15 min. (f, l, r) of the reaction. The images in the top row (a-f) represent the intermediates synthesized in the presence of poly A30 oligomers (SEQ ID NO: 4); the images in the second row (g-1) represent the intermediates synthesized in the presence of poly T30 oligomers (SEQ ID NO: 5); the images in the last row (m-r) represent the intermediates synthesized in the absence of DNA. Before initiation of the reduction reaction, 100 μL of 0.5 nM AuNS solution was incubated with 1 μM DNA. The scale bar indicates 20 nm.

FIG. 11 is a TEM image of small gold nanoparticles produced from the conversion of Au(I)-mercaptopropionic acid complexes into metal particles on the TEM grid upon electron-beam irradiation during TEM imaging. HAuCl₄ (167 μM) was mixed with mercaptopropionic acid (1.5 mM), and the mixture was dropped on the TEM grid. The TEM image was taken after the sample was dried. The scale bar indicates 20 nm.

FIG. 12 is a schematic illustration of one embodiment of a method for DNA-mediated shape control of gold nanoparticles. Poly A (SEQ ID NO: 4); Poly T (SEQ ID NO: 5); Poly C (SEQ ID NO: 6).

FIG. 13 depicts melting curves of the DNA on AuNFs (circles) and free DNA in solution (squares). Both melting curves were obtained using buffer containing 10 mM HEPES buffer (pH 7.1) and 50 mM NaCl.

FIGS. 14a-14d are TEM images of nanoassemblies: (a) AuNF_A30 with AuNS₅ₙₘ_{_{—}}S_T30; (b) AuNF_A30 with non-complementary AuNS₅ₙₘ_{_{—}}S_A30; (c) AuNS_T30 with AuNS₅ₙₘ_{_{—}}S_A30; (d) AuNS_T30 with non-complementary AuNS₅ₙₘ_{_{—}}S_T30. The scale bar indicates 20 nm.

FIGS. 15a-15d are TEM images of nanoassemblies: (a, b) AuNF_A30 with AuNS₅ₙₘ_{_{—}}S_T30; (c, d) AuNF_A30 with non-complementary AuNS₅ₙₘ_{_{—}}S_A30. The scale bar indicates 100 nm.

FIG. 16 depicts Raman spectra of the Raman tag (Trama) from AuNFs (upper line) and AuNSs (lower line). The samples were excited with 603 nm laser.

FIG. 17 is a dark-field light-scattering image of gold nanoflowers. The scale bar indicates 2 μm.

FIGS. 18a-18b are dark-field images of Chinese hamster ovary (CHO) cells (a) treated with AuNF particles, (b) without nanoparticle treatment. The scale bar indicates 10 μm.

FIGS. 19a-19h are optical and confocal fluorescence images of CHO cells treated with AuNF nanoparticles synthesized with FAM-A30 (19a-19d) or without nanoparticle treatment (19e-19h). FIG. 19a is a brightfield image of the AuNF treated cells; FIGS. 19b-19d are corresponding 3-D reconstructed confocal fluorescence images of the AuNF treated cells (19b: top view; 19c, 19d: side views; unit scale: 1 μm); FIG. 19e is a brightfield image of the control cells; FIGS. 19f-19h are corresponding 3-D reconstructed confocal fluorescence images of the control cells (19f: top view; 19g, 19h: side views; unit scale: 1 μm). The scale bars in FIGS. 19a and 19e indicate 10 μm. The AuNFs (1 nM) were incubated with CHO cells for 20 hours before imaging. The fluorescence arises from the incomplete quenching of fluorophore by the gold nanoparticles. It was shown that the fluorescent dots were distributed inside the cells, indicating that the AuNFs were taken up by the cells after incubation. As a comparison, the control cells without nanoparticle treatment showed little fluorescence.

FIGS. 20a-20d are TEM images of nanoparticles synthesized with A30 oligomers (SEQ ID NO: 4) (20a), T30 oligomers (SEQ ID NO: 5) (20b), C30 oligomers (SEQ ID NO: 6) (20c) and G10 oligomers (SEQ ID NO: 18) (20d) by using gold nanoprisms as seeds. FIGS. 21a-21c are TEM images of nanorod seeds before reaction (21a), and nanoparticles synthesized with A30 oligomers (SEQ ID NO: 4) (21b), and T30 oligomers (SEQ ID NO: 5) (21c) using the gold nanorod seeds.

FIGS. 22a-22d are TEM images of nanoflowers synthesized with increasing concentrations of gold.

FIGS. 23a-23b are graphs of size versus gold salt concentration, demonstrating a linear relationship between gold salt concentration and nanoflower size. The nanoflowers were synthesized with a randomized DNA construct (SEQ ID NO: 1) (23a) or an AS1411 aptamer (SEQ ID NO: 2) (23b); 50 particles were counted to determine size.

FIGS. 24a-24c are TEM images of gold nanoflowers grown from 15-nm, 30-nm, and 50-nm gold nanoparticles, respectively.

FIG. 25 is a graph illustrating the absorption spectra of gold nanoflowers grown from 15-nm, 30-nm, and 50-nm gold nanoparticles.

FIGS. 26a-26b are dark-field optical images of MCF-7 cells incubated with nanoflowers comprising control DNA (SEQ ID NO: 1) (26a) or nanoflowers comprising the AS1411 aptamer (SEQ ID NO: 2) (26b). The images were obtained under identical conditions and microscope settings.

FIG. 27 is a schematic diagram illustrating one embodiment of a method for making a DNA-shaped nanoparticle.

FIGS. 28a-28f are a series of scanning electron microscopy (SEM) images of gold nanoparticles prepared from gold nanoprisms with (28a) A30 oligomers (SEQ ID NO: 4), (28b) T30 oligomers (SEQ ID NO: 5), (28c) C30 oligomers (SEQ ID NO: 6), (28d) G20 oligomers (SEQ ID NO: 7), (28e) in the absence of DNA, (28f) in the absence of gold salt. The scale bar indicates 200 nm.

FIGS. 29a-29d are a series of SEM images of gold nanoparticles prepared from gold nanoprisms with oligomers of varying length from 5 bases to 30 bases: (29a) poly A series (SEQ ID NOs: 8-10, 4), (29b) poly T series (SEQ ID NOs: 11-13, 5), (29c) poly C series (SEQ ID NOs: 14-16, 6), (29d) poly G series (SEQ ID NOs: 17-19, 7). The scale bar indicates 200 nm.

FIGS. 30a-30d are a series of SEM images of gold nanoparticles prepared from gold nanoprisms in the presence of 60 μM of (30a) dAMP, (30b) dTMP, (30c) dCMP and (30d) dGMP. The scale bar indicates 200 nm.

FIGS. 31a-31d are UV-Vis-NIR absorbance spectra illustrating the time-dependent absorbance evolution of gold nanoparticle growth solutions in the presence of (31a) A20 (SEQ ID NO: 10), (31b) T20 (SEQ ID NO: 13), (31c) C20 (SEQ ID NO: 16), and (31d) G20 (SEQ ID NO: 7). The absorbance was monitored for 15 minutes with a time interval of 1 minute.

FIG. 32 is a series of SEM images of nanoparticle intermediates grown with A20 (SEQ ID NO: 10), T20 (SEQ ID NO: 13), C20 (SEQ ID NO: 16), and G20 (SEQ ID NO: 7). The nanoparticle growth was stopped with 1.5 mM mercaptopropionic acid at different time points. The scale bar indicates 200 nm.

FIGS. 33a-33f are a series of SEM images of gold nanoparticles synthesized with DNA containing two types of deoxyribonucleotides. (33a) A, T combinations: T5A25 (SEQ ID NO: 20), T10A20 (SEQ ID NO: 21), T15A15 (SEQ ID NO: 22), T20A10 (SEQ ID NO: 23), T25A5 (SEQ ID NO: 24) (from left to right); (33b) A, C combinations: A5C25 (SEQ ID NO: 25), A10C20 (SEQ ID NO: 26), A15C15 (SEQ ID NO: 27), A20C10 (SEQ ID NO: 28), A25C5 (SEQ ID NO: 29) (from left to right); (33c) A, G combinations: A5G25 (SEQ ID NO: 30), A10G20 (SEQ ID NO: 31), A15G15 (SEQ ID NO: 32), A20G10 (SEQ ID NO: 33), A25G5 (SEQ ID NO: 34) (from left to right). (33d) T, C combinations: T5C25 (SEQ ID NO: 35), T10C20 (SEQ ID NO: 36), T15C15 (SEQ ID NO: 37), T20C10 (SEQ ID NO: 38), T25C5 (SEQ ID NO: 39) (from left to right); (33e) T, G combinations: T5G25 (SEQ ID NO: 40), T10G20 (SEQ ID NO: 41), T15G15 (SEQ ID NO: 42), T20G10 (SEQ ID NO: 43), T25G5 (SEQ ID NO: 44) (from left to right); (33f) C, G combinations: C5G25 (SEQ ID NO: 45), C10G20 (SEQ ID NO: 46), C15G15 (SEQ ID NO: 47), C20G10 (SEQ ID NO: 48), C25G5 (SEQ ID NO: 49) (from left to right). The scale bar indicates 200 nm.

FIG. 34 is a diagram illustrating the representative nanoparticles produced by different DNA sequences containing one or two types of deoxyribonucleotides and their interplay effects.

FIG. 35 is an SEM image of gold nanoparticles synthesized with a DNA oligomer having the sequence T10A10T10 (SEQ ID NO: 50). The scale bar indicates 200 nm.

FIGS. 36a-36b are a series of SEM images of gold nanoparticles synthesized with (36a) T20G5 (SEQ ID NO: 51), T20G10 (SEQ ID NO: 43), T20G15 (SEQ ID NO: 52) and T20G20 (SEQ ID NO: 53) (from left to right); (36b) A20G5 (SEQ ID NO: 54), A20G10 (SEQ ID NO: 33), A20G15 (SEQ ID NO: 55) and A20G20 (SEQ ID NO: 56) (from left to right). The scale bar indicates 200 nm.

FIGS. 37a-37b are SEM images of gold nanoparticles synthesized with (12a) T20G10A10 (SEQ ID NO: 57), and (12b) A10T20C10 (SEQ ID NO: 58). The scale bar indicates 200 nm.

FIG. 38 is a flowchart illustrating a decision process for selecting an oligomer sequence based on a nanoseed with a nanoprism morphology and a desired nanoparticle morphology.

FIG. 39 is a flowchart illustrating one embodiment of an investigative method for determining a genetic rule.

### SEQUENCE LISTING

The nucleic acid sequences provided herein are shown using standard letter abbreviations for nucleotide bases as defined in 37 C.F.R. 1.822. The sequence listing is submitted as an ASCII text file, named “Sequence.txt,” created on Dec. 17, 2012, ˜16 KB, which is incorporated by reference herein.

SEQ ID NO: 1 is a randomized control DNA sequence.

SEQ ID NO: 2 is a DNA sequence including the AS1411 aptamer sequence.

SEQ ID NO: 3 is a randomized DNA sequence.

SEQ ID NO: 4 is a Poly A sequence, A30.

SEQ ID NO: 5 is a Poly T sequence, T30.

SEQ ID NO: 6 is a Poly C sequence, C30.

SEQ ID NO: 7 is a poly G sequence, G20.

SEQ ID NO: 8 is a poly A sequence, A5.

SEQ ID NO: 9 is a poly A sequence, A10.

SEQ ID NO: 10 is a poly A sequence, A20.

SEQ ID NO: 11 is a poly T sequence, T5.

SEQ ID NO: 12 is a poly T sequence, T10.

SEQ ID NO: 13 is a poly T sequence, T20.

SEQ ID NO: 14 is a poly C sequence, C5.

SEQ ID NO: 15 is a poly C sequence, C10.

SEQ ID NO: 16 is a poly C sequence, C20.

SEQ ID NO: 17 is a poly G sequence, G5.

SEQ ID NO: 18 is a poly G sequence, G10.

SEQ ID NO: 19 is a poly G sequence, G15.

SEQ ID NO: 20 is a poly A/T sequence, T5A25.

SEQ ID NO: 21 is a poly A/T sequence, T10A20.

SEQ ID NO: 22 is a poly A/T sequence, T15A15.

SEQ ID NO: 23 is a poly A/T sequence, T20A10.

SEQ ID NO: 24 is a poly A/T sequence, T25A5.

SEQ ID NO: 25 is a poly A/C sequence, A5C25.

SEQ ID NO: 26 is a poly A/C sequence, A10C20.

SEQ ID NO: 27 is a poly A/C sequence, A15C15.

SEQ ID NO: 28 is a poly A/C sequence, A20C10.

SEQ ID NO: 29 is a poly A/C sequence, A25C5.

SEQ ID NO: 30 is a poly A/G sequence, A5G25.

SEQ ID NO: 31 is a poly A/G sequence, A10G20.

SEQ ID NO: 32 is a poly A/G sequence, A15G15.

SEQ ID NO: 33 is a poly A/G sequence, A20G10.

SEQ ID NO: 34 is a poly A/G sequence, A25G5.

SEQ ID NO: 35 is a poly T/C sequence, T5C25.

SEQ ID NO: 36 is a poly T/C sequence, T10C20.

SEQ ID NO: 37 is a poly T/C sequence, T15C15.

SEQ ID NO: 38 is a poly T/C sequence, T20C10.

SEQ ID NO: 39 is a poly T/C sequence, T25C5.

SEQ ID NO: 40 is a poly T/G sequence, T5G25.

SEQ ID NO: 41 is a poly T/G sequence, T10G20.

SEQ ID NO: 42 is a poly T/G sequence, T15G15.

SEQ ID NO: 43 is a poly T/G sequence, T20G10.

SEQ ID NO: 44 is a poly T/G sequence, T25G5.

SEQ ID NO: 45 is a poly C/G sequence, C5G25.

SEQ ID NO: 46 is a poly C/G sequence, C10G20.

SEQ ID NO: 47 is a poly C/G sequence, C15G15.

SEQ ID NO: 48 is a poly C/G sequence, C20G10.

SEQ ID NO: 49 is a poly C/G sequence, C25G5.

SEQ ID NO: 50 is a poly T/A sequence, T10A10T10.

SEQ ID NO: 51 is a poly T/G sequence, T20G5.

SEQ ID NO: 52 is a poly T/G sequence, T20G15.

SEQ ID NO: 53 is a poly T/G sequence, T20G20.

SEQ ID NO: 54 is a poly A/G sequence, A20G5.

SEQ ID NO: 55 is a poly A/G sequence, A20G15.

SEQ ID NO: 56 is a poly A/G sequence, A20G20.

SEQ ID NO: 57 is a poly T/G/A sequence, T20G10A10.

SEQ ID NO: 58 is a poly A/T/C sequence, A10T20C10.

SEQ ID NO: 59 is a DNA sequence of aptamer S2.2.

SEQ ID NO: 60 is a DNA sequence of aptamer TLS11a-GC.

SEQ ID NO: 61 is a DNA sequence of aptamer sgc8.

### DETAILED DESCRIPTION

Embodiments of a method for making shaped nanoparticles by adsorbing nucleic acid oligomers onto a metal nanoseed and depositing metal onto the oligomer-functionalized nanoseed are disclosed. Embodiments of a method for determining a shape effect rule, a surface characteristic effect rule, or both based on variations in a nucleic acid oligomer sequence composition and the resulting shaped nanoparticle's morphology also are disclosed. The nucleic acid oligomers may be DNA, RNA, or PNA; the oligomers also may include modified bases. In some embodiments, a metal nanoparticle with a desired morphology is made by adsorbing nucleic acid strands onto a nanoseed and then reducing metal onto the nanoseed, wherein the nanoparticle growth pattern and morphology is determined by the nucleic acid sequence and/or the relative abundance of individual nucleotides in the nucleic acid strand.

Nanoparticles made by some embodiments of the disclosed method can be taken up by cells. Because metallic nanoparticles can be visualized by, e.g., darkfield microscopy, such nanoparticles may be useful for intracellular imaging. Additionally, nanoparticles that can be taken up by cells may be useful carriers for delivering drugs, contrast agents, genes, and other molecules into cells.

### I. TERMS AND ABBREVIATIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Definitions of common terms in chemistry may be found in Richard J. Lewis, Sr. (ed.), Hawley's Condensed Chemical Dictionary, published by John Wiley & Sons, Inc., 1997 (ISBN 0-471-29205-2). All references herein are incorporated by reference. In order to facilitate review of the various embodiments of the disclosure, the following explanations of specific terms are provided:

Administration: To provide or give a subject an agent, such as a nanoparticle preparation described herein, by any effective route. Exemplary routes of administration include, but are not limited to, topical, injection (such as subcutaneous, intramuscular, intradermal, intraperitoneal, intratumoral, and intravenous), oral, sublingual, rectal, transdermal, intranasal, ocular, vaginal and inhalation routes.

Adsorption: The physical adherence or bonding of ions and molecules onto the surface of another molecule or substrate. An ion or molecule that adsorbs is referred to as an adsorbate. Adsorption can be characterized as chemisorption or physisorption, depending on the character and strength of the bond between the adsorbate and the substrate surface. Chemisorption is characterized by a strong interaction between an adsorbate and a substrate, e.g., formation of covalent and/or ionic bonds. Physisorption is characterized by weaker bonding between an adsorbate and a substrate. The weaker bond typically results from van der Waals forces, i.e., an induced dipole moment between the adsorbate and the substrate.

Antibody: A polypeptide ligand comprising at least a light chain or heavy chain immunoglobulin variable region which specifically recognizes and binds an epitope of an antigen, such as a tumor-specific protein. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997

Typically, a naturally occurring immunoglobulin has heavy (H) chains and light (L) chains interconnected by disulfide bonds. There are two types of light chain, lambda (2) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

Antibodies also include polyclonal antibodies.

Aptamer: An oligonucleic acid that binds to a specific target. Nucleic acid aptamers are capable of binding to various molecular targets such as small molecules, proteins, nucleic acids, or cells. DNA or RNA aptamers recognize target effector molecules with high affinity and specificity (Ellington and Szostak, Nature 346(6287):818-822, 1990; Tuerk and Gold, Science, 249:505-510, 1990). Aptamers have several unique properties. First, aptamers for a given target can be obtained by routine experimentation. For instance, in vitro selection methods can be used (called systematic evolution of ligands by exponential enrichment (SELEX)) to obtain aptamers for a wide range of target effector molecules with exceptionally high affinity, having dissociation constants in the picomolar range (Brody and Gold, Reviews in Molecular Biotechnology, 74(1)5-13, 2000, Jayasena, Clinical Chemistry, 45(9):1628-1650, 1999, Wilson and Szostak, Ann. Rev. Biochem., 68:611-647, 1999, Ellington et al., Nature 1990, 346, 818-822; Tuerk and Gold Science 1990, 249, 505-510; Liu et al., Chem. Rev. 2009, 109, 1948-1998; Shamah et al., Acc. Chem. Res. 2008, 41, 130-138; Famulok, et al., Chem. Rev. 2007, 107, 3715-3743; Manimala et al., Recent Dev. Nucleic Acids Res. 2004, 1, 207-231; Famulok et al., Acc. Chem. Res. 2000, 33, 591-599; Hesselberth, et al., Rev. Mol. Biotech. 2000, 74, 15-25; Morris et al., Proc. Natl. Acad. Sci. U.S.A. 1998, 95, 2902-2907). Second, aptamers are easier to obtain and less expensive to produce than antibodies, because aptamers can be generated in vitro in short time periods (for example, within days) and at economical cost. Third, aptamers display remarkable structural durability and can be denatured and renatured many times without losing their ability to recognize their targets. The mononucleotides of an aptamer may adopt a particular conformation upon binding to its target. Aptamers that are specific to a wide range of targets from small organic molecules such as adenosine, to proteins such as thrombin, and even viruses and cells, have been identified (Chou et al., Trends in Biochem Sci. 2005, 30(5), 231-234; Liu et al., Chem. Rev. 2009, 109, 1948-1998; Lee et al., Nucleic Acids Res. 2004, 32, D95-D100; Navani and Li, Curr. Opin. Chem. Biol. 2006, 10, 272-281; Song et al., TrAC, Trends Anal. Chem. 2008, 27, 108-117; Tombelli et al., Bioelectrochemistry, 2005, 67(2), 135-141). In one example the aptamer is specific for HIV (such as HIV-tat).

Contacting: Placement in direct physical association, including both a solid and liquid form. Contacting can occur in vitro, for example, with isolated cells, such as tumor cells, ex vivo, for example using a sample obtained from a patient (e.g., a biopsy sample, fine needle aspirate, or the like), or in vivo by administering to a subject (such as a subject with a tumor). Thus, the nanoparticles disclosed herein can be contacted with cells in vivo, ex vivo, or in vitro, under conditions that permit the nanoparticle to bind to or to enter the cell (for example via endocytosis.

DNA melting temperature: The temperature at which a DNA double helix dissociates into single strands, specifically the temperature at which 50% of the DNA, or oligonucleotide, is in the form of a double helix and 50% has dissociated into single strands. The most reliable and accurate determination of melting temperature is determined empirically. Methods for determining the melting temperature of DNA are known to those with ordinary skill in the art of DNA characterization. For single-stranded oligomers, a complementary oligonucleotide is hybridized to the oligomer, and the melting temperature of the double-stranded complex is determined.

Nanobowl: A nanoparticle with a morphology in microscopic view that resembles a bowl, i.e., the edges are thicker than the center portion of the nanoparticle.

Nanoflower (NF): A nanoparticle with a morphology in microscopic view that resembles a flower.

Nanohexagon: A nanoparticle with a hexagonal morphology in microscopic view.

Nanoparticle (NP): A nanoscale particle with a size that is measured in nanometers, for example, a particle that has at least one dimension of less than about 100 nm. Nanoparticles may have different shapes, e.g., nanofibers, nanoflowers, nanohorns, nano-onions, nanopeanuts, nanoplates, nanoprisms, nanorods, nanoropes, nanospheres, nanostars, nanotubes, etc.

Nanoplate: A nanoparticle with a morphology in microscopic view that resembles a substantially flat plate. The plate can have a rough surface or a smooth surface.

Nanoprism: A nanoparticle with a morphology in microscopic view that resembles a prism, i.e., a polyhedron with two congruent and parallel faces (the bases) and whose lateral faces are parallelograms. In some embodiments, the bases are triangular.

Nanoseed (NS): A small nanoparticle used as a starting material for larger nanoparticle synthesis. For example, gold ions may be reduced and deposited onto gold nanoseeds to produce larger gold nanoparticles.

Nanostar: A nanoparticle with a morphology in microscopic view that resembles a star.

Near-infrared (NIR): The infrared spectrum is typically divided into three sections, with near-infrared including the shortest wavelengths. Although the region is not rigidly defined, NIR typically encompasses light with wavelengths ranging from 700-2000 nm.

Oligomer: A general term for a polymeric molecule consisting of relatively few monomers, e.g., 5-100 monomers. In one example, the monomers are nucleotides or ribonucleotides.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of the nanoparticles disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A polymer is a molecule of repeating structural units (e.g., monomers) formed via a chemical reaction, e.g., polymerization.

“Specifically binds” refers to the ability of a molecule to bind with specificity to a particular target. For example, “specifically binds” refers to the ability of an individual aptamer to specifically bind to a molecular target such as a small molecule, a protein, a particular nucleic acid sequence, or a particular cell.

“Specifically binds” also refers to the ability of individual antibodies to specifically immunoreact with an antigen, such as a tumor-specific antigen, relative to binding to unrelated proteins, such as non-tumor proteins, for example β-actin. For example, a HER2-specific binding agent binds substantially only the HER-2 protein in vitro or in vivo. As used herein, the term “tumor-specific binding agent” includes tumor-specific antibodies and other agents that bind substantially only to a tumor-specific protein in that preparation.

The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody”.

In some examples, an antibody (such as an antibody conjugated to a nanoparticle of the present disclosure) specifically binds to a target (such as a cell surface protein) with a binding constant that is at least 10³ M⁻¹ greater, 10⁴M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 1 nM or less. For example, an antibody binds to a target, such as tumor-specific protein with a binding affinity of at least about 0.1×10⁻⁸M, at least about 0.3×10⁻⁸M, at least about 0.5×10⁻⁸M, at least about 0.75×10⁻⁸M, at least about 1.0×10⁻⁸M, at least about 1.3×10⁻⁸M at least about 1.5×10⁻⁸M, or at least about 2.0×10⁻⁸M. Kd values can, for example, be determined by competitive ELISA (enzyme-linked immunosorbent assay) or using a surface-plasmon resonance device such as the Biacore T100, which is available from Biacore, Inc., Piscataway, N.J.

Subject or patient: A term that includes human and non-human mammals. In one example, the subject is a human or veterinary subject, such as a mouse.

Therapeutically effective amount: An amount of a composition that alone, or together with an additional therapeutic agent(s) (such as a chemotherapeutic agent) sufficient to achieve a desired effect in a subject, or in a cell, being treated with the agent. The effective amount of the agent (such as the nanoparticles disclosed herein) can be dependent on several factors, including, but not limited to the subject or cells being treated, the particular therapeutic agent, and the manner of administration of the therapeutic composition. In one example, a therapeutically effective amount or concentration is one that is sufficient to prevent advancement, delay progression, or to cause regression of a disease, or which is capable of reducing symptoms caused by the disease, such as cancer. In one example, a therapeutically effective amount or concentration is one that is sufficient to increase the survival time of a patient with a tumor.

In one example, a desired response is to reduce or inhibit one or more symptoms associated with cancer. The one or more symptoms do not have to be completely eliminated for the composition to be effective. For example, administration of a composition containing a nanoparticle disclosed herein, which in some examples is followed by photothermal therapy can decrease the size of a tumor (such as the volume or weight of a tumor, or metastasis of a tumor) by a desired amount, for example by at least 20%, at least 50%, at least 80%, at least 90%, at least 95%, at least 98%, or even at least 100%, as compared to the tumor size in the absence of the nanoparticle. In one particular example, a desired response is to kill a population of cells by a desired amount, for example by killing at least 20%, at least 50%, at least 60%, at least 70%, at least 80%, at least 90%, at least 95%, at least 98%, or even at least 100% of the cells, as compared to the cell killing in the absence of the nanoparticle. In one particular example, a desired response is to increase the survival time of a patient with a tumor (or who has had a tumor recently removed) by a desired amount, for example increase survival by at least 20%, at least 50%, at least 60%, at least 70%, at least 80%, at least 90%, at least 95%, at least 98%, or even at least 100%, as compared to the survival time in the absence of the nanoparticle.

The effective amount of the disclosed nanoparticles that is administered to a human or veterinary subject will vary significantly depending upon a number of factors associated with that subject, for example the overall health of the subject. An effective amount of an agent can be determined by varying the dosage of the product and measuring the resulting therapeutic response, such as the regression of a tumor. Effective amounts also can be determined through various in vitro, in vivo or in situ immunoassays. The disclosed agents can be administered in a single dose, or in several doses, as needed to obtain the desired response. However, the effective amount of the disclosed nanoparticles can be dependent on the source applied, the subject being treated, the severity and type of the condition being treated, and the manner of administration. In certain examples, a therapeutically effective dose of the disclosed nanoparticles is at least 20 mg per kg body weight, at least 200 mg per kg, at least 2,000 mg per kg, or at least 20 g per kg, for example when administered intravenously (iv).

In particular examples, a therapeutically effective dose of an antibody conjugated to a nanoparticle of the present disclosure is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered iv. However, one skilled in the art will recognize that higher or lower dosages also could be used, for example depending on the particular nanoparticle. In particular examples, such daily dosages are administered in one or more divided doses (such as 2, 3, or 4 doses) or in a single formulation. The disclosed nanoparticle can be administered alone, in the presence of a pharmaceutically acceptable carrier, in the presence of other therapeutic agents (such as other anti-neoplastic agents).

Treating: A term when used to refer to the treatment of a cell or tissue with a therapeutic agent, includes contacting or incubating an agent (such as a nanoparticle disclosed herein) with the cell or tissue. A treated cell is a cell that has been contacted with a desired composition in an amount and under conditions sufficient for the desired response. In one example, a treated cell is a cell that has been exposed to a nanoparticle under conditions sufficient for the nanoparticle to enter the cell, which is in some examples followed by phototherapy, until sufficient cell killing is achieved.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated with the claimed nanoparticles include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma.

The disclosed nanoparticles can also be used to treat liquid tumors, such as a lymphatic, white blood cell, or other type of leukemia.

Under conditions sufficient for: A phrase that is used to describe any environment that permits the desired activity. In one example, “under conditions sufficient for” includes administering a nanoparticle to a subject sufficient to allow the nanoparticle to enter the cell. In particular examples, the desired activity is killing the cells into which the nanoparticles entered, for example following phototherapy of the cells. In another example, “under conditions sufficient for” includes contacting DNA oligomers with a nanoseed sufficient to allow the oligomers to bind to the nanoseed, to form a nanoparticle of the desired shape.

### II. NANOPARTICLE PREPARATION AND NUCLEIC ACID-MEDIATED SHAPE CONTROL

The disclosure provides shaped nanoparticles having attached thereto nucleic acid oligomers. A novel “genetic code” for designing nanoparticles having a particular morphology is also disclosed. By selecting and using particular combinations of oligonucleotides, nanoparticles having a particular morphology, such as a particular shape and/or surface, can be generated. Certain “rules” for controlling the shape and surface characteristics of a nanoparticle by using particular nucleic acid sequences are identified. Thus, DNA, RNA, or PNA oligomers can be used to control the shape of a nanoparticle to which they are attached. Also provided are methods of making and using such shaped nanoparticles, and methods of determining additional “rules” for controlling the shape and surface characteristics of a nanoparticle.

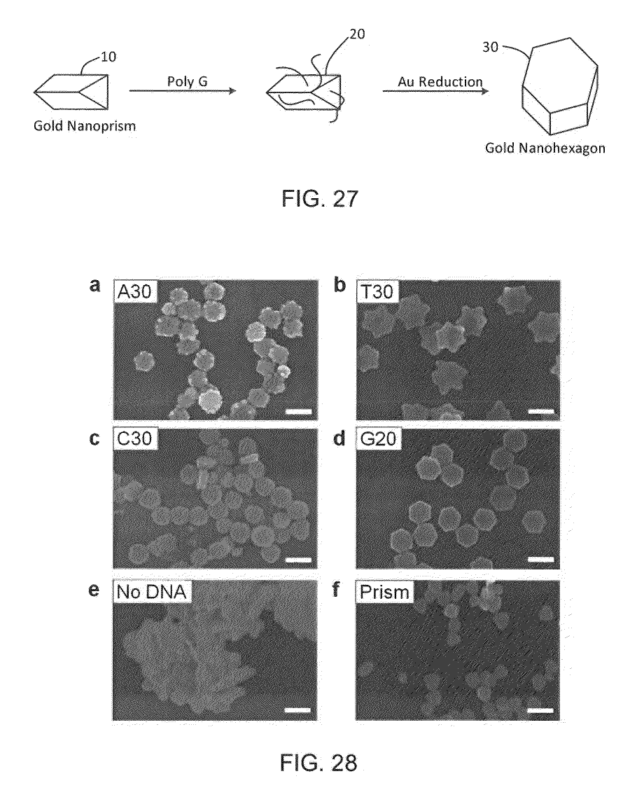

Embodiments of the shaped nanoparticles are made by adsorbing a plurality of nucleic acid oligomers onto a metal nanoseed to produce an oligomer-functionalized nanoseed, and then depositing additional metal, e.g., by reduction, onto the functionalized nanoseed to produce a shaped nanoparticle. The nanoparticle's morphology is determined, at least in part, by the nanoseed shape and the nucleic acid sequence of the oligomers. A brief overview of one embodiment of a method for producing a nucleic acid-shaped gold nanoparticle is illustrated in FIG. 27. A gold nanoprism 10 is used as the nanoseed. The gold nanoprism 10 is combined with a plurality of poly G oligomers (e.g., G30) to produce an oligomer-functionalized nanoprism 20. Additional gold is reduced and deposited onto the oligomer-functionalized nanoprism 20, thereby producing a gold nanohexagon 30.

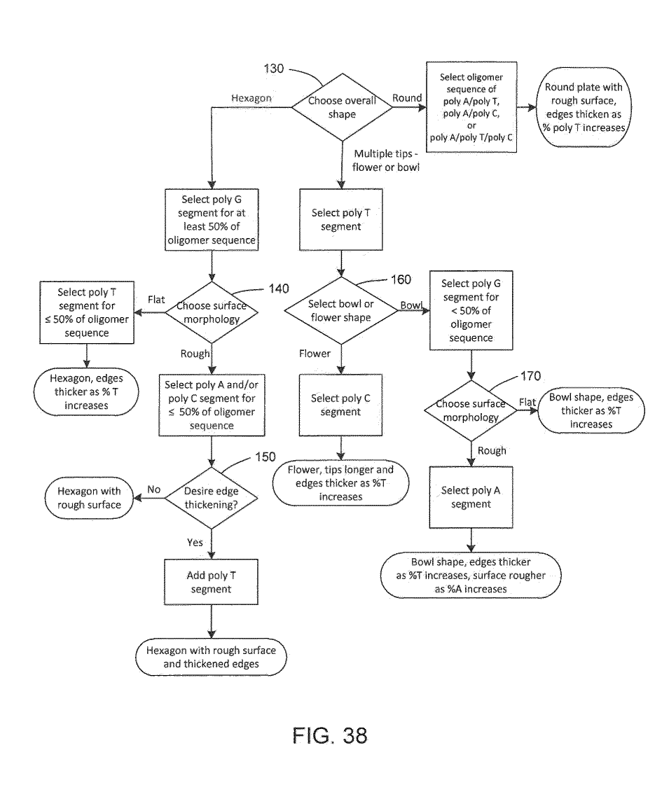

FIG. 38 is a flowchart illustrating one embodiment of a decision process for selecting a genetic code, i.e., an oligomer sequence, to produce a desired morphology for a nanoparticle prepared from an oligomer-functionalized nanoprism. In some embodiments, the oligomer sequence includes at least two different sequence segments, each segment being at least five nucleotides in length and including a single type of nucleobase selected from A, C, T (or U), and G. In step 130, an overall shape is selected. If the selected shape is a hexagon, a poly G segment is selected for at least 50% of the oligomer sequence, such as at least 65%, at least 80%, 50-100%, 50-80%, or 50-90% of the oligomer sequence.

In step 140, a surface morphology (i.e., flat or rough) is selected for the hexagon. Surface “roughness” refers to a surface that is irregular, uneven, bumpy, and/or textured. A rough surface has ridges, irregularities, and/or protrusions that project from a surface baseline. In some embodiments, the ridges, irregularities, and/or protrusions project at least 2 nm, such as 2-100 nm, from the surface baseline. A “flattened” surface refers to a surface that is uniform, even, smooth, and/or lacking texture. In one example, a flattened surface varies by less than 2 nm from the surface baseline. If a flattened surface is selected, a poly T segment is selected for the remainder of the oligomer sequence (such no more than 10%, no more than 20%, no more than 50%, 5-50%, 10-40%, or 20-40% of the oligomer sequence). Poly T also produces edge thickening, and the hexagon will have thicker edges as the percent poly T increases. “Edge” refers to an outer portion of the surface, e.g., the portion of the surface having a radius that is at least 50%, at least 70%, at least 90%, or at least 95% of the nanoparticle's average radius, such as the portion having a radius that is 50-100%, 70-100%, 90-100%, or 95-100% of the nanoparticle's average radius. “Thicker” edges means the edges have an average thickness that is greater than the average thickness of the nanoparticle's center portion (i.e., the portion having a radius less than 50% of the nanoparticle's average radius). For example, the edges may have a thickness that is at least 1.1 times, at least 1.3 times, at least 1.5 times, or at least 2 times the average thickness of the nanoparticle's center portion, or the edges may have an average thickness at least 5 nm greater, at least 10 nm greater, or at least 25 nm greater than the nanoparticle's average center portion thickness. If a rough surface is chosen, a poly A segment, a poly C segment, or a combination thereof is selected for the oligomer sequence. Both A and C interact competitively with G, and the surface roughness will increase proportionally as the poly A or poly C segment length increases from at least 5 nucleobases in length to less than 50% of the oligomer sequence (such no more than 10%, no more than 20%, no more than 50%, 5-50%, 10-40%, or 20-40% of the oligomer sequence). However, the surface roughness effect generally reaches a maximum when the percentage of poly A or poly C is greater than 80% of the oligomer sequence.

In step 150, a decision is made concerning edge thickness of the hexagon. If thickened edges relative to the nanoparticle's center thickness are desired, a poly T segment is added to the oligomer sequence (i.e., the oligomer sequence will comprise three segments—a poly G segment, a poly C segment, and a poly T segment). As the percent poly T increases, the thickness of the edges will increase. If poly T is not included, the hexagon will have an average edge thickness that is substantially similar to its average center thickness.

If a morphology comprising multiple tips (e.g., a flower morphology or a six-pointed bowl-like morphology) is chosen in step 130, a poly T segment is selected for the oligomer sequence. In step 160, a decision is made between a six-pointed bowl shape or a flower shape. If a flower shape is selected, a poly C segment is added to the poly T segment. Poly C and poly T interact synergistically, and a flower morphology is achieved for all ratios of C:T as long as each sequence segment is at least 5 nucleobases in length. However, as the percent poly T increases relative to the percent poly C in the oligomer, the flower tips will become longer and the edges will become thicker relative to the center thickness.

If a six-pointed bowl shape is chosen at step 160, a poly G segment is selected for less than 50% of the oligomer sequence, such as less than 35% or less than 25% of the oligomer length. A combination of poly T and poly G produces a six-pointed bowl with thickened edges relative to the center thickness. As the percent poly T in the oligomer increases relative to the percent poly G, the edge thickness will increase.

At step 170, a decision is made between a smooth surface and a roughened surface. If a roughened surface is selected, a poly A segment is selected for the oligomer. A has a dominating effect over T and G. Thus, the poly A segment can be relatively short, e.g., at least five nucleobases in length, but less than 25% of the oligomer length, such as less than 20% or less than 10%.

If a round morphology is chosen in step 130, an oligomer sequence comprising a combination of poly A and poly T segments, a combination of poly A and poly C segments, or a combination of poly A, poly C, and poly T segments is selected. Because the poly A effect dominates, all combinations of A and C will produce a round nanoplate with a rough surface. As the percent poly T increases, the nanoplate edges will thicken relative to the nanoplate center. If the percent poly T reaches 80% or greater, the poly T may exert a flattening effect on the surface in addition to thickening the nanoplate edges.

In some embodiments, the nanoseeds (NSs), or starting materials, for nanoparticle growth are nanoprisms. In other embodiments, the nanoseeds are, e.g., nanospheres or nanorods. A person of ordinary skill in the art of nanoparticle technology will understand that NSs of any shape may be used; however, the final nanoparticle's morphology may depend at least in part upon the shape of the NS. Nanoseeds may comprise any material to which nucleic acid oligomers can be attached. If the nanoparticles will be administered to living subjects, it can be advantageous to prepare nanoparticles (NPs) that do not have significant cellular toxicity. In particular embodiments, gold NPs are produced from gold NSs. Gold has very low cellular toxicity, and thus can be used for applications in living subjects. Other suitable materials include other metals, such as silver and platinum, as well as inorganic compounds (e.g., silica, metal oxide).

Typically, the nanoseeds have a largest dimension, or diameter, between 1 nm to 1000 nm, such as from 1 nm to 25 nm, 1 nm to 50 nm, 1 nm to 100 nm, 1 nm to 250 nm, 1 nm to 500 nm, 5 nm to 20 nm, 5 nm to 50 nm, 5 nm to 100 nm, 5 nm to 150 nm, 10 nm to 50 nm, 10 nm to 100 nm, 10 nm to 500 nm, 10 nm to 1000 nm. In some embodiments, AuNSs with a diameter of 1-200 nm are used.

In some embodiments, nucleic acid oligomers comprising a single type of nucleobase (or base) are used (e.g., poly A). In other embodiments, the oligomers include more than one type of base (such as an oligomer containing any combination of A, T, U, C, and/or G). Thus, the nucleic acid oligomers can include one, two, three, four, or five different bases. In some embodiments, the oligomers comprise a particular nucleobase sequence. In other embodiments, the oligomers include a particular composition, or percentage, of nucleobases, e.g., 20% A and 80% T.

A nucleobase (or base) is a nitrogen-based molecule that forms the basic building blocks of DNA, RNA, and peptide nucleic acid (PNA) oligomers and polymers. DNA and RNA nucleosides are formed when a nucleobase is bonded to deoxribose or ribose, respectively.

A nucleotide is a nucleoside bonded to one or more phosphate groups. PNA is formed from repeating N-(2-aminoethyl)-glycine units linked by peptide bonds, wherein the nucleobases are linked to the backbone by methylene carbonyl bonds. Nucleobases provide the molecular structure necessary for the hydrogen bonding of complementary DNA, RNA, and PNA strands. The primary nucleobases are cytosine, guanine, adenine (DNA, RNA, PNA), thymine (DNA, PNA) and uracil (RNA), abbreviated as C, G, A, T, and U, respectively. Adenine and guanine belong to the double-ringed class of molecules called purines. Cytosine, thymine, and uracil are pyrimidines.

If the nucleic acid oligomer is a DNA molecule, then reference to “A”, “G”, “T” or “C” herein refers to nucleotides comprising deoxyadenosine, deoxyguanosine, deoxycytidine or deoxythymidine, respectively. If the nucleic acid oligomer is a RNA molecule, then reference to “A”, “G”, “C” or “U” herein refers to nucleotides comprising adenosine, guanosine, cytidine or uridine, respectively. If the nucleic acid oligomer is a PNA molecule, then reference to “A”, “G”, “T” or “C” herein refers to an adenine, guanine, thymine, and cytosine, respectively, linked to an N-(2-aminoethyl)-glycine unit by a methylene carbonyl bond.

As an alternative to using nucleobases, modified nucleobases, including modified nucleotides or ribonucleotides, can be used. Modified nucleobases include those nucleotides containing modified bases, modified sugar moieties and/or modified phosphate backbones. Examples of modified base moieties which can be used in the methods herein, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, N6-methyladenine, 1-methylguanine, 1-methylinosine, 2,2-dimethylguanine, 2-methyladenine, 2-methylguanine, 3-methylcytosine, 5-methylcytosine, 5-hydroxymethylcytosine, β-D-glucosyl-5-hydroxymethylcytosine, 5-hydroxymethyluracil, N6-adenine, 1-methylguanine, 7-methylguanine, 7-methyladenine, 5-methylaminomethyluracil, methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, 5,6-dihydrouracil, 2-aminopurine, 2,6-diaminopurine, 6-thioguanine, ribothymidine, and 7-chloro-1,8-naphthyridin-2-(1H)-one.

Examples of modified sugar moieties which may be used to modify nucleotides or ribonucleotides include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Oligomers containing five or more bases are suitable for use in the disclosed embodiments. An oligomer is a general term for a polymeric molecule consisting of relatively few monomers, e.g., 5-100 monomers. In one example, the monomers are nucleotides or ribonucleotides. Oligomers with fewer than five bases are too short to significantly influence the nanoparticle morphology. The oligomers disclosed herein can be at least 5 bases in length, such as at least 10, at least 20, at least 30, or at least 40 bases in length, such as 5 to 100 bases in length, 5 to 40 bases, or 5 to 30 bases in length. In some embodiments, all of the oligomers bound to the NS have the same sequence and the same length. In other embodiments, oligomers of differing sequences and/or differing lengths may be used.

In some embodiments, the oligomers may be modified or labeled with a detectable label. Suitable detectable labels may include, but are not limited to, fluorophores (e.g., fluorescein dyes, Alexa Fluor® dyes, etc.), radioisotopes, biotin, photo-sensitive linkers, and chemical functional groups (e.g., alkynyl, azide, carboxyl, etc.).

In some embodiments, gold nanoseeds are coated with citrate during nanoseed synthesis. Oligomers adsorb to the citrate-coated AuNS surface via physisorption. In some embodiments, gold nanoprisms are used as the nanoseeds. Gold nanoprisms are single crystalline with {111} facets on the top and bottom flat surfaces, and twin planes and stacking faults on the side faces. Thus, gold nanoprisms are capable of both vertical and horizontal growth.

After nucleic acid (NA) oligomers are adhered to the NS surface, additional material is deposited onto the nucleic acid-nanoseed (NA-NS) construct to produce nanoparticle growth. In some embodiments, hydrogen tetrachloroaurate(III) (HAuCl₄) was used as a gold ion source to deposit additional gold onto the NA-NS construct. However, other soluble gold salts also can be used. Hydroxylamine (NH₂OH) is a suitable reducing agent for reducing HAuCl₄ catalyzed by the gold surface.³⁵ Other reducing agents also may be used, e.g., ascorbic acid, amines (poly(allylamine) hydrochloride⁵¹, sodium diphenylamine sulfonate⁵²), and the like.

Nanoparticle size is controlled by varying the nanoseed size and/or the growth conditions. For example, the amount of HAuCl₄ can be limited and controlled to precisely control the size of the resulting nanoparticle. In certain embodiments, the nanoparticle has a largest dimension, or diameter of 5-1,000 nm, such as 10-500 nm, 10-250 nm, or 20-200 nm. Typically, particles with a largest dimension between 20 nm and 200 nm are suitable for in vivo applications. Nanoparticle size is a result-effective variable that may influence uptake activity for nanoparticles having a particular shape, surface functionalization, and/or environment.

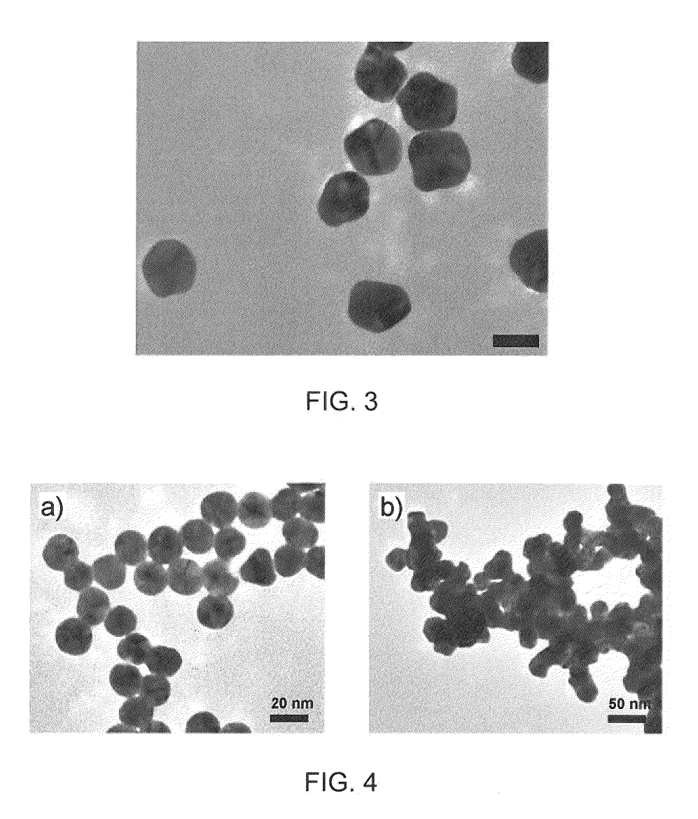

The nanoseed shape, nucleic acid sequence, and/or nucleic acid composition (relative amounts of each nucleotide) affect the nanoparticle morphology. For example, gold nanoparticles synthesized from gold nanospheres with adsorbed A30 and C30 are flower shaped (FIGS. 2a-2b), while nanoparticles synthesized with adsorbed T30 oligomers are spherical (FIG. 2c). Nanoparticles synthesized from nanospheres in the absence of DNA also are spherical (FIG. 2d), as are nanoparticles synthesized in the presence of a 10-mer of poly G (FIG. 3).

Nanoparticles synthesized from gold nanoprisms incubated with poly A30, poly T30, poly C30, or poly G20 form round nanoplates with bumpy surfaces (FIG. 28a), six-pointed nanostars (FIG. 28b), round nanoplates with smooth surfaces (FIG. 28c), and hexagonal nanoplates (FIG. 28d), respectively. Gold nanoparticles synthesized with each type of DNA sequence are uniformly shaped and mono-dispersed. In contrast, when nanoprisms are grown in the absence of DNA, but under the same growth conditions, the nanoparticles produced are larger than micro-sized gold agglomerations and have irregular shapes (FIG. 28e). In contrast, if the purified nanoprisms are incubated with DNA in the absence of a reducing agent or gold salts under otherwise identical conditions, minimal change in the particle morphology is observed (FIG. 28f). Thus, it is apparent that the nucleic acid sequence mediates nanoparticle growth and controls the resulting shape of the nanoparticle.

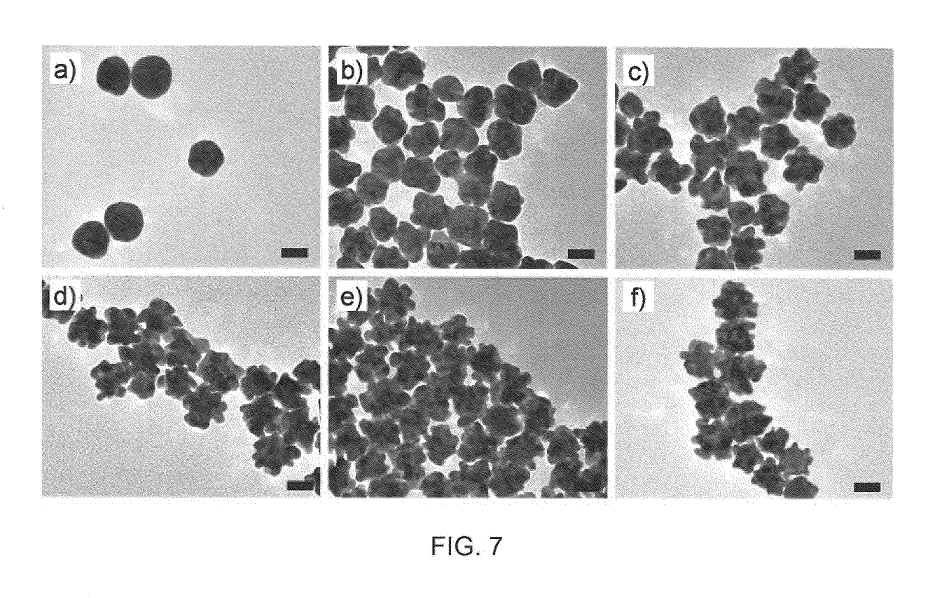

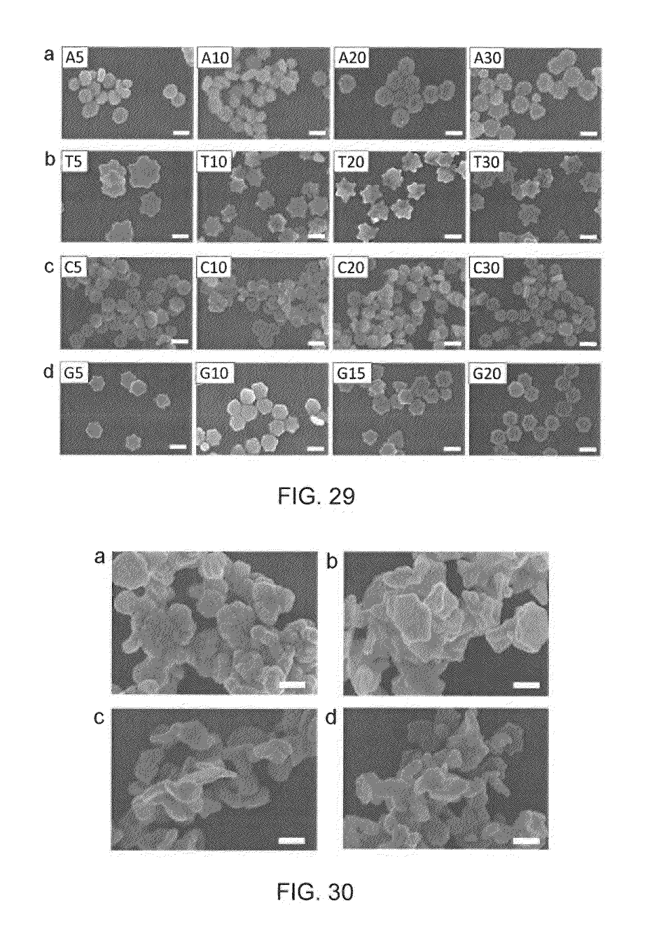

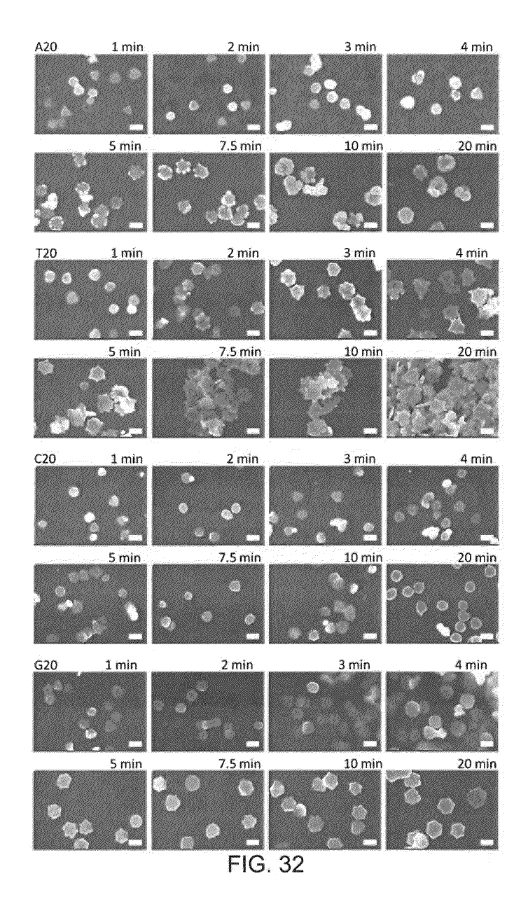

The length and number of oligomers adsorbed to the nanoseed also significantly affect the nanoparticle shape. To affect morphology, the nucleic acid oligomer has at least five bases. Nucleic acid oligomers with fewer bases are not long enough to significantly influence the nanoparticle morphology. However, as shown in FIGS. 29a-29d, when the oligomer length is extended from 5 to 20 bases, or from 5 to 30 bases, the shape remains substantially the same. Thus, different DNA bases, regardless of oligomer length, direct the growth of the nanoprism into different shapes based on the identity of the DNA nucleotide. In some embodiments, the morphology changes over time as the nanoparticle grows (FIG. 32). For example, nanoprisms incubated with T20 initially grow into hexagonal plates and then into six-pointed nanostars. Nanoprisms incubated with G20 first grow into round nanoplates and then into nanohexagons. Furthermore, the number of oligomers adsorbed to the nanoseed significantly affects the nanoparticle morphology. As shown in FIGS. 7a-f, shape control becomes increasingly evident as the number of oligomers increases.

Different nucleobases (e.g., nucleotides) have different binding affinities on gold surfaces. For instance A, C, and G have a stronger binding affinity for gold than T.^{62,63} Thus, different nucleic acid sequences interact differently with the nanoseeds, and shape them into different morphologies during growth. In some embodiments, two of more different bases combined in a nucleic acid oligomer interact in a synergistic, competitive, or additive manner to shape the nanoparticle and produce a morphology that differs from the morphologies produced by oligomers including only one type of base. In another embodiment, one type of base may exert a dominant effect over another base, thereby producing a nanoparticle having a morphology characteristic of a single base.

Thiol chemistry can be used to conjugate DNA, RNA, and PNA to gold surfaces. However, when thiolated (i.e., thiol-modified) DNA was adsorbed onto gold nanospheres (see Example 2), all of the thiolated DNA was displaced by mercaptoethanol. In contrast, embodiments of AuNFs produced with unmodified poly A oligonucleotides by the methods disclosed herein were resistant to mercaptoethanol displacement, and incubation with mercaptoethanol overnight displaced less than one-third of the DNA strands. Thus, some embodiments of the disclosed in situ synthesis and controlled reduction methods advantageously can be used to prepare stable nucleic acid-functionalized gold surfaces. Certain embodiments of shaped gold nanoparticles produced by the disclosed methods are very stable in aqueous solution, even in the presence of 0.3 M salt, demonstrating that nucleic acid oligomers can be attached to the nanoparticles during their synthesis, and act as stabilizing ligands.

Considering the remarkably high binding affinity of nucleic acid oligomers to the AuNPs (higher than thiol-gold binding), it was hypothesized that the oligomers in situ attached to AuNPs during reduction could be partially buried in the shaped nanoparticles. As additional gold is deposited onto the oligomer-functionalized nanoseed, a portion of the oligomer strand becomes buried in the deposited gold, thereby firmly attaching the oligomer to the nanoparticle during nanoparticle growth. Because the melting point of an oligonucleotide bound to a complementary oligonucleotide increases with the length of the oligonucleotide, an attached oligonucleotide may have a lower melting point than that of a free oligonucleotide if a portion of the attached oligonucleotide is buried within the gold nanoparticle. In some embodiments, the attached oligonucleotides have a melting point that is at least 10% or at least 20% (such as 10-20%) lower than that of the corresponding free oligonucleotides, substantiating the hypothesis that a portion of the oligonucleotide strand is embedded within the gold nanoparticle during nanoparticle growth. In certain embodiments, it is preferable to control nanoparticle size by varying the nanoseed size rather than by varying the thickness of the deposited gold. Varying the nanoseed size while minimizing the thickness of the deposited gold allows minimal “trapping” of the oligomer sequence by the growing gold layer.

In some embodiments, it is beneficial to maximize the morphology while minimizing the thickness of the deposited gold. Growth can be monitored by the nanoparticle's UV absorbance. For example, gold spherical nanoparticles exhibit specific UV absorbance in the 500-600 nm range, and the absorption at this wavelength is a good indicator of the size and the polydispersity of the nanoparticles. As spherical nanoseeds grow into nanoflowers, the absorption peak will blue shift (increase in wavelength) and the absorbance at the original wavelength will decrease. By monitoring the subsequent shifted peak that corresponds to the formation of the nanoflower structure as well as the original peak of the nanosphere, it is possible to assign a quality factor to track the growth of nanoflowers that is expressed as:

Quality factor=Abs<Nanoflower>/Abs<Nanosphere>

The optimum gold concentration that maximizes nanoflower morphology with minimum gold growth can be determined by plotting this quality factor vs. the amount of gold salt added.

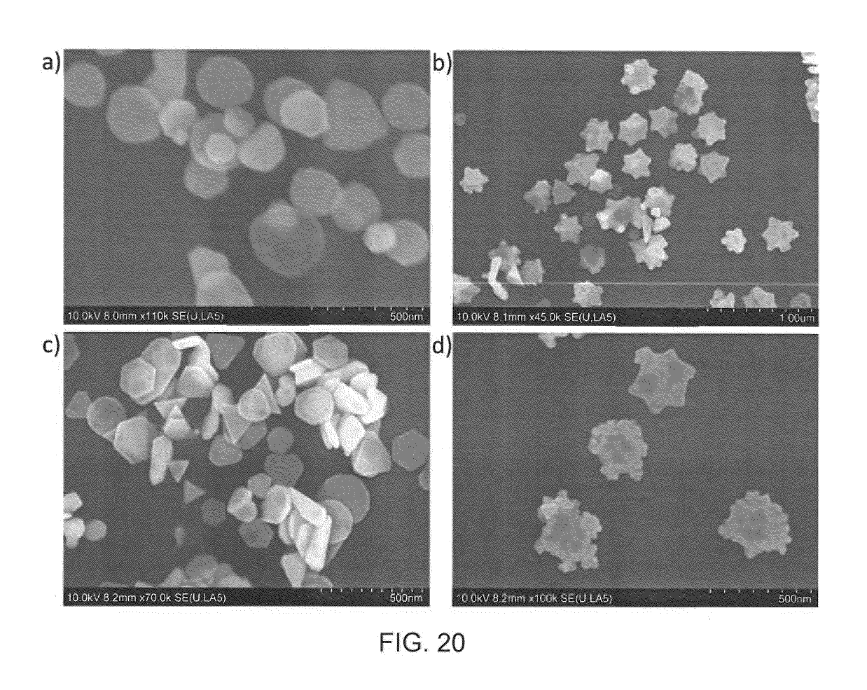

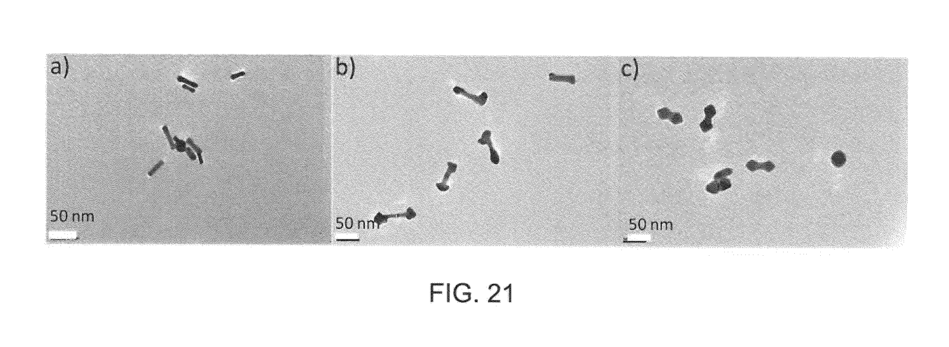

The sequence of the nucleic acid also mediates growth and morphology of nanoparticles synthesized from non-spherical seeds. In a working embodiment, when gold nanoprisms were functionalized with A30 or C30 DNA oligomers and additional gold was deposited, flat nanoplates were formed (FIGS. 20a, 20c). Thus, DNA oligomers of poly A or poly C, or a mixture of A and C (such as a DNA oligomer of at least 75% A and C), can be attached to gold nanoprisms to make flat nanoplates. In contrast, gold nanoprisms functionalized with T30 or G10 DNA oligomers formed multi-pointed nanostars (FIG. 20b, 20d). Thus, DNA oligomers of poly T or poly G, or a mixture of T and G (such as a DNA oligomer of at least 75% T and G), can be attached to gold nanoprisms to make multi-pointed nanostars. In another working embodiment, gold nanorods functionalized with A30 DNA oligomers produced bone-shaped, or dumbbell-shaped, nanoparticles (FIG. 21b), whereas nanorods functionalized with T30 oligomers produced nanoparticles resembling peanuts (FIG. 21c). Thus, DNA oligomers of poly A, or a mixture of A with other nucleotides (such as a DNA oligomer of at least 75% A), can be attached to gold nanorods to make dumbbell-shaped, nanoparticles, while DNA oligomers of poly T, or a mixture of T with other nucleotides (such as a DNA oligomer of at least 75% T), can be attached to gold nanorods to make nanoparticles resembling peanuts.

In certain embodiments, a nucleic acid sequence is selected based at least in part on its ability to bind to a target, e.g., a target protein. In such embodiments, it is desirable to control nanoparticle size by selecting an appropriately sized nanoseed and then depositing a thin layer of gold so that only a minimal portion of the oligomer is buried in the deposited gold. For example, aptamer AS1411 (SEQ ID NO: 2) recognizes and binds to nucleolin, a eukaryotic nucleolar phosphoprotein involved in the synthesis and maturation of ribosomes. In order to facilitate binding to its target, the entire AS1411 sequence preferably is fully exposed. Thus, in some embodiments, the nanoseed is functionalized with a plurality of oligomers comprising the aptamer plus an additional “tail” of nucleotides, e.g., a poly C tail, such that a portion of the tail is embedded in the deposited metal while the aptamer sequence remains fully exposed. Thus, in one example, an aptamer sequence is attached at its 3′-end to the 5′-end of the oligomer (e.g., DNA, RNA, or PNA) sequence selected to obtain the desired shape/structure. In another example, an aptamer sequence is attached at its 5′-end to the 3′-end of the oligomer (e.g., DNA, RNA, or PNA) sequence selected to obtain the desired shape/structure. Based on this teaching, one can select an appropriate aptamer based on the target, and incorporate the selected aptamer into the disclosed nanoparticles.

In some embodiments, the method is investigative. Varying the oligomer sequence composition, the nanoseed shape, and/or the reaction conditions (e.g., concentration of the metal ion source, reaction temperature, reaction time, etc.) may produce variations in the resulting nanoparticle morphology. “Genetic rules” can be determined by evaluating changes in the nanoparticle morphology and correlating the changes with the variations in the oligomer sequence composition, the nanoseed shape, and/or the reaction conditions. A genetic rule may be a shape effect rule, a surface characteristic effect rule, or both. The rules can be used, singly or in combination, to predict morphologies of nanoparticles formed from other oligomer sequences.

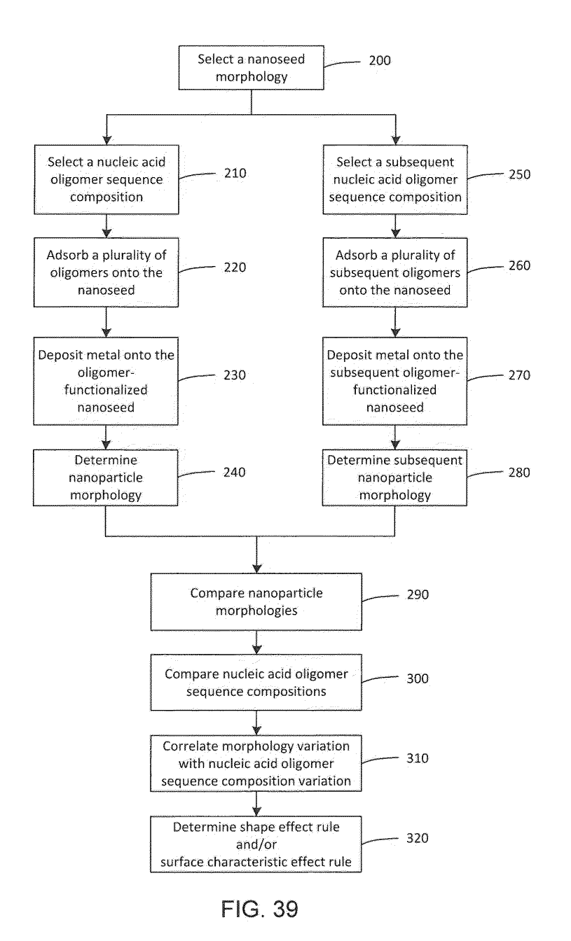

FIG. 39 is a flowchart illustrating one embodiment of an investigative method for determining a genetic rule based upon variations in the nucleic acid oligomer sequence composition. In step 200, a nanoseed morphology is selected. In step 210, a nucleic acid oligomer sequence composition is selected. In certain embodiments, the nucleic acid oligomer has an oligomer sequence composition comprising at least two unique sequence segments, each sequence segment comprising one type of nucleobase selected from the group consisting of A, C, G, T, and U. In step 220, a plurality of nucleic acid oligomers is adsorbed onto the nanoseed to produce an oligomer-functionalized nanoseed, and additional metal is reduced onto the oligomer-functionalized nanoseed in step 230 to produce a shaped nanoparticle. In step 240, the nanoparticle morphology is determined by visualizing the nanoparticle, e.g., by TEM or SEM. In step 250, a subsequent nucleic acid oligomer sequence composition is selected. The subsequent nucleic acid oligomer sequence composition has a variation in the length of at least one sequence segment, the type of nucleobase in at least one sequence segment, or both, compared to the first nucleic acid oligomer sequence composition. In step 260, a plurality of the subsequent nucleic acid oligomers is adsorbed onto the nanoseed to produce a subsequent oligomer-functionalized nanoseed, and additional metal is reduced onto the subsequent oligomer-functionalized nanoseed in step 270 to produce a subsequent shaped nanoparticle. The morphology of the subsequent nanoparticle is determined in step 280. The nanoparticle morphologies are compared in step 290, and the nucleic acid oligomer sequence compositions are compared in step 300. In step 310, the morphology variation is correlated with the nucleic acid oligomer sequence composition variation. Based on the correlation, a shape effect rule, a surface characteristic effect rule, or both is determined in step 320.

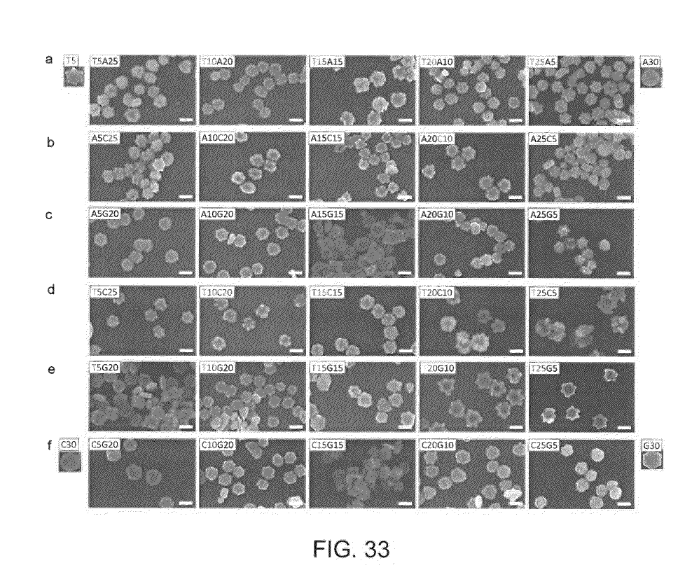

Several rules were determined by varying the nucleic acid oligomer sequence composition and determining subsequent nanoparticle morphologies as described in Example 13 below and illustrated in FIGS. 33a-33f. For instance, it was determined that A has a dominating effect over T. AT combinations produce round nanoplates with bumpy surfaces, similar to those produced by poly A alone. A and C have similar effects when present in oligomers containing two types of bases. AC combinations produce round nanoplates with bumpy surfaces. A and G compete with one another, producing a nanoparticle morphology determined by the nucleotide present in greater amount (FIG. 33c). When the sequence is predominantly A, the resulting morphology is a round, bumpy nanoplate. When the sequence is predominantly G, however, the nanoparticle morphology is a hexagonal plate. C and G also interact competitively (FIG. 33f) to form round, bumpy nanoplates when C is dominant and nanohexagons when G is dominant. T and C have a synergistic effect when combined, and form a new shape that is different from the shapes produced by either poly T or poly C. Whereas poly T produces six-pointed nanostars and poly C produces round, smooth nanoplates, an oligomer sequence combining T and C produces nanoflowers with thin central areas and thick edges having multiple tips (FIG. 33d). T and G combine with a competitive effect (FIG. 33e) to produce nanoparticles with characteristics attributable to both T and G. T-G oligomer sequences that have a large percentage of G, i.e., at least 50%, produce nanohexagons, similar to poly G. However, as the percentage of T increases and the percentage of G falls below 50%, the morphology transforms into a bowl-shaped, six-pointed nanostar as the edges thicken and develop a star shape.

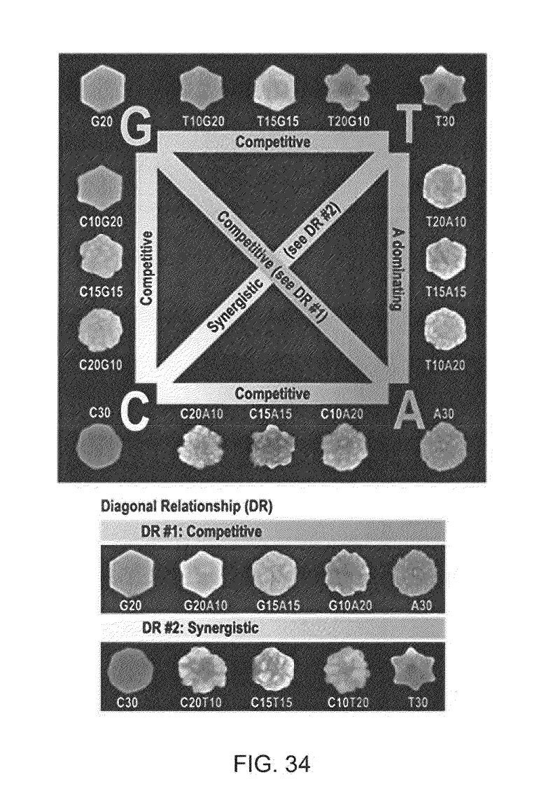

By comparing the newly formed nanoparticle morphologies with the morphology of the original nanoprism seeds, the shaping and surface characteristic effects (or genetic rules) of G, T, A, C in poly G, poly T, poly A, poly C by themselves or in combinations were identified (see Table 1). Since the poly G series could shape the original nanoprism seeds into flat nanohexagons, the shape effect of G is “formation of a flat nanohexagon.” In the case of polydeoxyribonucleotides containing two types of bases, G produced nanoparticles with flat surfaces, and directed the formation of hexogonal plates; thus the shape effect of G is “hexagon forming” and its surface effect is “flattening.” G's morphological effects are clear in examples of TG, AG, and CG combinations (FIG. 34). The poly T series produced six-pointed nanostars with smooth surfaces and thicker edges; thus, T's shaping effect is “six-pointed star forming” and its surface effect is “smoothing, edge thickening.” Such shaping effects by T have been demonstrated in TC and TG combinations. Poly A induces the formation of round nanoplates with rough surfaces, and thus its shape effect is “rounding” and its surface effect is “roughening.” These effects were confirmed in AT, AG, and AC combinations. The one exception in these predictions by base is the effect of poly C. Even though poly C was shown to induce round nanoplates with flat surfaces, once combined with T or G (as in the case of TC and GC combinations), C induced the formation of rough nanoplates; thus, its morphological effects were similar to those of A in combination with other bases.

TABLE 1

| | Shaping effect in | Shaping and surface |
|---|---|---|
| | DNA containing | characteristic effects in |
| | one type of | DNA containing two types |
| Nucleotide | deoxyribonucleotide | of deoxyribonucleotides |
| A | Formation of rough | Rounding, surface |
| | round plates | roughening |
| T | Formation of 6- | Formation of 6-pointed |
| | pointed nanostars | stars, surface smoothing, |
| | | edge thickening |
| G | Formation of flat | Formation of nanohexagons, |
| | nanohexagons | surface flattening |
| C | Formation of flat | Rounding, surface |
| | round plates | roughening |

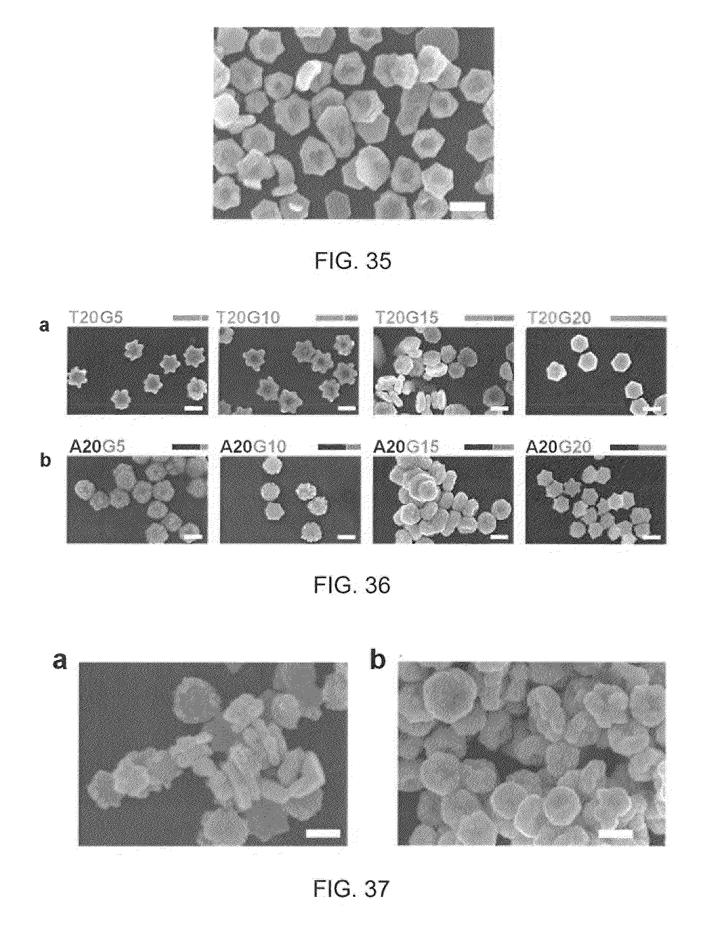

The shaping effects of A, T, C, and G, and the interplay between the bases, are summarized in FIG. 34. In TG, AG, CG and AC combinations, the morphological influence of each deoxyribonucleotide competed directly with one another, and the longer segment dominated the shaping process. The AT combinations were also competitive, but the A segment's shaping effect dominated even if it was shorter than the T segment. T's morphological effect could be enhanced by placing poly T segment at both ends of the 30 mer DNA. For example, T10A10T10 produced nanoplates with thick edges and thin centers; this result could be attributed to the morphological effects of the T segments (FIG. 35). The TC combinations showed a synergistic interaction, producing a flower-like nanoparticle. T and C enhanced the growth effects of the nanoparticles, producing large petals on the sides of the plates and creating a morphology distinctly different from either the poly T or poly C case (FIG. 34).

Thus, in some embodiments, application of the genetic rules to the nucleobase sequence used during synthesis predicts nanoparticle morphology and/or surface roughness. In one embodiment, surface roughness is enhanced by including an A segment and/or a C segment in the DNA oligomer. In another embodiment, a hexagonal morphology is produced by including a G segment in the DNA oligomer. G segments also flatten surfaces, and increasing the length of the G segment can smooth surface roughness. In yet another embodiment, edges can be flattened and/or thickened by including a T segment.

In some embodiments, the morphology of a nucleic acid-shaped nanoparticle is predicted based on the nanoseed morphology, the relationships outlined in FIG. 34, and/or the shape and surface characteristic effect rules shown in Table 1. For example, a nanoparticle synthesized from a nanoprism functionalized with an oligomer comprising T, G, and A nucleotides can be predicted to have flattening and edge-thickening from T, surface roughening from A, and a flat hexagonal shape from G, producing a roughly hexagonal nanoparticle with a thin center, thick edges, and a bumpy surface (see, e.g., FIG. 37a). In another embodiment, a nanoparticle synthesized from a nanoprism functionalized with an oligomer comprising A, T, and C can be predicted to have flattening and edge-thickening from T, and surface roughening from A and C, producing a rounded, donut-shaped nanoparticle with a thin center, thick edges, and a rough surface (FIG. 37b).

In some embodiments, a nanoseed is provided and a desired nanoparticle morphology is selected based at least in part on the nanoseed's morphology. Based on the nanoseed morphology, the relationships outlined in FIG. 34, and/or the shape and surface characteristic effects shown in Table 1, a suitable oligomer sequence can be selected to produce a nanoparticle having the desired morphology.

In some examples, the disclosed nanoparticles further include other molecules. In one example, the disclosed nanoparticles further include antibodies or fragments thereof that can be used to target a nanoparticle to a target cell. In one example, the antibody is specific for a cell surface receptor, such as a receptor on a cancer cell. Such nanoparticles can be used for example to image or treat (e.g., kill) the cancer cell. In another example, the disclosed nanoparticles further include a therapeutic molecule that can be used to treat a target cell. For example, the therapeutic molecule can be a drug that is used to treat a disease, such as a chemotherapeutic agent (e.g., cisplatin, doxorubicin, fluorouracil). In another example, the therapeutic molecule is a nucleic acid molecule used for gene therapy.

Chemotherapeutic agents are known in the art (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Exemplary chemotherapeutic agents that can be conjugated to a nanoparticle provided herein include but are not limited to, carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone and vinorelbine.

### III. NANOPARTICLE USES

Bio-functionalization of nanomaterials can provide the nanomaterials with target recognition ability, and can enable their controlled assembly.⁴¹ This functionalization step typically involves chemical modifications of the nanoparticles or the biomolecules to allow conjugation. For example, some embodiments of the disclosed shaped nanoparticles (e.g., nanoflowers, nanoplates, nanospheres, and/or nanostars), are capable of binding to and/or entering a target cell. In one embodiment, a nucleic-acid functionalized nanoparticle includes a molecule of interest (e.g., an antibody, antibody fragment, peptide, protein, aptamer, drug molecule, or nucleic acid molecule [for example a therapeutic nucleic acid, such as a DNA or RNA molecule, such as an mRNA molecule, inhibitory RNA molecule (such as an siRNA or miRNA), gene, cDNA, or gene fragment]). For example, the molecule of interest may be conjugated to the nucleic acid oligomer extending from the nanoparticle (for example from the 5′-end or 3′-end of the oligomer) or the molecule of interest may be conjugated directly to the nanoparticle surface. Certain embodiments of the disclosed shaped nanoparticles are capable of forming larger nano-assemblies comprising a plurality of shaped nanoparticles. Additionally, some embodiments of the disclosed shaped nanoparticles have unique optical and/or electrical properties that may provide utility for imaging and/or biosensing applications, e.g., surface-enhanced Raman spectroscopy-based biosensing.

Nucleic acid-shaped nanoparticles (NPs) have several advantages over nanospheres. For example, nanoflowers, nanostars, and nanoplates with bumpy surfaces have a higher surface area than nanospheres of a similar size. Therefore, more biomolecules or drug can be loaded on each NP. In addition, the tips of nanoscale protrusions and nanocavities on the surface of some embodiments of the disclosed nucleic acid-shaped gold nanoparticles (AuNPs) have strong localized near-field enhancement effects, and they give a much stronger Raman signal enhancement effect than gold nanospheres. Furthermore, preliminary studies indicate that some shaped NPs, e.g., gold nanoflowers, are more easily taken up and internalized by cells via endocytosis than non-functionalized gold nanospheres.

In some embodiments, nucleic acid-shaped nanoparticles also have different optical properties than nanospheres. For example, gold nanospheres have a maximum absorbance at 520-530 nm, whereas round nanoplates with bumpy surfaces (synthesized with A20) have a maximum absorbance at about 800 nm (FIG. 31a), and six-pointed nanostars synthesized with T20 have a maximum absorbance at about 1200 nm (FIG. 31b). The absorbance shift allows visualization of some nucleic acid-shaped nanoparticles with near-infrared or infrared radiation, and also may make nucleic acid-shaped nanoparticles suitable candidates for photothermal therapies since near-IR absorption increases the temperature of the nanoparticles.

Nucleic acid-functionalized nanoflowers may be used as imaging agents and nano-carriers in a cellular environment. For example, some embodiments of nucleic acid-shaped nanoparticles, e.g., DNA-functionalized AuNFs, can be taken up by cells. Without being bound by any particular theory, it is believed that this cellular uptake ability might be due to high DNA loading on the AuNP surface and/or the morphology of the AuNP. Intracellular AuNPs scatter light and can be visualized, for example, using dark-field microscopy. The cellular uptake ability and light scattering property allow the AuNPs to be used as nano-carriers for drug or gene delivery and contrast agents for intracellular imaging.

In certain embodiments, a nucleic-acid functionalized nanoparticle comprises an aptamer capable of binding to an antigen of interest. Nucleic acid aptamers have been shown to be a useful targeting ligand for many biologically and medically relevant targets, and have shown potential for in vivo targeting applications.^{57,58} Thus, an aptamer-functionalized nanoparticle can be used to deliver the nanoparticle to a desired target (such as a particular cell type, for example a cancer cell). In one example the aptamer is specific for a protein on a cancer cell, such as aptamer S2.2 (5′-GCA GTT GAT CCT TTG GAT ACC CTG G-3′; SEQ ID NO: 59) specific for breast cancer cells (see Ping et al., Anal. Chem., 2012, 84:7692-7699), aptamer TLS11a-GC (5′-ACAGCATCCCCATGTGAACAATCGCATTGTGATTG TTACGGTTTCCGCCTCATGGACGTGCTG-3′; SEQ ID NO: 60) specific for hepatocellular carcinoma (see Meng et al., PLOS One, 7:e33434, 2012), or aptamer sgc8 (TAA CTG CTG CGC CGC CGG GAA AAT ACT GTA CGG TTA GAT TTT TTT TTT; SEQ ID NO: 61) for T-cell acute lymphoblastic leukemia (see Shangguan et al., J. Proteome Res. 7:2133-9, 2008 and Phillips et al. Anal. Chem. 81:1033-9, 2009). In one embodiment, a gold nanoflower comprises an AS1411 aptamer, which binds specifically to nucleolin, a protein that is over expressed ˜20-fold on the surface of certain cancer cells and is an exemplary binding target for human breast cancer cells, e.g., MCF-7.

Molecules of interest (e.g., antibodies, peptides, proteins, aptamers, drug molecules, mRNA molecules, siRNA molecules, genes, or gene fragments) may be attached to nucleic acid-shaped gold nanoparticles by conventional coupling techniques. For example, molecules of interest can be attached to nucleic acid-shaped AuNPs by conventional gold or nucleic acid coupling techniques. In some embodiments, the nucleic acid oligomers may be chemically modified to facilitate functionalization with the molecules of interest. In other embodiments, the molecules of interest may be attached directly to the NP surface.

Nanoparticle-antibody conjugates may be used to deliver the nucleic acid-shaped nanoparticles to desired targets. For example, an antibody that recognizes a particular target antigen on a cell surface may be conjugated to the NP. Alternatively, a first antibody specific for a target antigen may be administered where it binds to the target antigen. Subsequently, a NP conjugated to a second antibody that recognizes and binds to the first antibody is administered, thereby delivering the NP to the target. For example, a mouse monoclonal antibody specific for a target antigen may be administered to a subject where it binds to the target antigen, followed by administration of an anti-mouse antibody-NP conjugate.

In one embodiment, an antibody-NP conjugate may be used for detecting and/or imaging target cells. For example, antibodies to an antigen found on the surface of cancer cells may be conjugated to nucleic acid-shaped nanoparticles. The antibody-NPs may be administered to a subject, with the antibody then recognizing and binding to the cancer cell antigens. The cancer cells may be detected and/or imaged by any suitable method, such as CT or x-ray imaging. Alternatively, the cells can be analyzed ex vivo. For example, a sample from the patient can be obtained, and then contacted with the antibody-NPs under conditions that allow the antibody-NPs to bind to a target in the sample, and such binding visualized or detected (e.g., using microscopy). In one embodiment, an antibody-NP conjugate may be used in photothermal and/or radiotherapy, e.g., for treatment of cancer. Photothermal therapy is a technique that converts electromagnetic radiation (usually in the form of infrared) into thermal energy as a therapeutic technique for medical conditions, such as cancer. Gold and silver nanoparticles have emerged as powerful platforms for in vitro, ex vivo, and in vivo biomedical applications, due to their high stability, low toxicity, and ability to be taken up by cells.⁵⁹ As the dimensions decrease in metals, the properties of the surface become dominant and give nanoparticles new properties. In noble metals, the coherent collective oscillation of electrons in the conduction band induces large surface electric fields which greatly enhance the radiative properties of gold and silver nanoparticles when they interact with resonant electromagnetic radiation. This makes the absorption cross section of these nanoparticles orders of magnitude stronger than that of the most strongly absorbing molecules and the light scattering cross section orders of magnitude more intense than that of organic dyes. It was realized that this intense absorption provided a path to efficiently convert IR light to an intense local heating around the nanoparticle.⁶⁰ Photothermal therapy places these metal nanoparticles only in and around diseased and/or cancerous cells to create localized heating that would selectively kill the targeted cells without damaging the surrounding area.⁶¹

In order to be considered applicable for in vivo applications, nanoparticles should absorb EM radiation most efficiently from 700 nm to 900 nm, also known as the near IR window where skin, tissues, and hemoglobin have minimum absorption and scattering, allowing the radiation to penetrate deep into the tissue. The efficiency with which a nanoparticle can convert near IR radiation to thermal energy is partly determined by electric fields that arise from the oscillations of surface electrons. Sharp, pointed features, such as the morphological features of nanoflowers and nanostars, behave as focusing points for such oscillations and can dramatically increase the radiative properties at these locations.

Some embodiments of the disclosed nucleic acid-shaped nanoparticles absorb energy in the near-infrared region. For example, gold nanoflowers, bumpy round nanoplates, smooth round nanoplates, and nanohexagons have been shown to absorb energy in the near-infrared region (see, e.g., FIGS. 31a, 31b, 31d). Absorption of NIR energy will increase the temperature of the AuNP. Thus, an antibody-AuNP conjugate bound to a cancer cell may be irradiated with NIR radiation, thereby heating the nanoparticle and destroying the cancer cell. Alternatively, the AuNPs may be used to increase the dose of x-ray radiation received by the cancer cells relative to the dose received by normal tissue. The absorption characteristics of AuNPs may allow effective treatment (e.g., cancer cell destruction) with less radiation than conventional gold nanospheres.

In other embodiments, nucleic acid-shaped nanoparticles may be used to deliver molecules of interest (e.g., a drug, peptide, protein, aptamer, or a nucleic acid molecule, such as a DNA or RNA molecule, for example an mRNA molecule, miRNA molecule, cDNA molecule, siRNA molecule, gene, or gene fragment) to a cell. For example, a therapeutic drug molecule may be conjugated to the NP surface or to the nucleic acid oligomers protruding from the NP surface. Because cells can take up nucleic acid-functionalized AuNPs, such as gold nanoflowers (see Example 6), the AuNP may be used to deliver a drug molecule to the cell interior. Alternatively, coupling a molecule of interest to an NP-antibody conjugate may be used to deliver the molecule of interest to the immediate environment, or vicinity, of a targeted cell. Thus, an anti-cancer drug-NP-antibody conjugate, for example, could be delivered specifically to a tumor site rather than disseminated throughout the body. Such methods can be used in combination with other therapies, such as other anti-neoplastic therapies, such as radiation therapy, chemotherapy immunosuppressants (such as Rituximab, steroids), cytokines (such as GM-CSF, IL-4), and members of the Bcl-2 family (such as Bax or Bad).

A gene or gene fragment may be conjugated to a nucleic acid-shaped nanoparticle to deliver the gene or gene fragment into a cell for gene therapy (for example to increase expression of a desired protein), or for detection of complementary DNA in the cell. Similarly, an inhibitory RNA-nanoparticle conjugate (such as an siRNA-nanoparticle or miRNA-nanoparticle conjugate) may be used to deliver the inhibitory RNA for therapy, e.g., to decrease or silence expression of a specific disease-related gene or to treat a viral infection.

Nanoparticles prepared by embodiments of the disclosed method also can be used to make nano-assemblies. Nucleic acid-directed nano-assemblies may be used for biosensing and nanoscale photonic device applications. A nucleic acid-shaped nanoparticle can be functionalized with oligomers of a given sequence. If at least a portion of the oligomer sequence extends from the nanoparticle surface, or additional oligomers are adsorbed to the shaped nanoparticle's surface, the exposed portions of the oligomers can act as ligands to bind and attach additional nanoparticles to which complementary oligomers are attached. For example, nanoparticles functionalized with oligomers comprising a poly T segment that extends from the nanoparticle surface can bind to a nanoparticles functionalized with oligomers comprising a poly A segment that extends from the nanoparticle surface via the interaction between the poly A and poly T oligomers. However, if the added nanoparticles include non-complementary oligomers, then little or no binding occurs. For example, nanoparticles with bound poly A oligomers will not bind to another poly A-NP. Thus, formation of the nano-assemblies is sequence specific. Additionally, the number of oligomers on the “central” nanoparticle determines in part how many “peripheral” nanoparticles including complementary oligomers can be attached to form the nano-assembly. As the number of oligomers on the central nanoparticle increases, so does the number of peripheral nanoparticles that can assemble onto it. One of ordinary skill in the art will understand that the number of nanoparticles in the nano-assembly also depends at least in part upon space constraints and the relative sizes of the nanoparticles. A larger central nanoparticle can accommodate more peripheral nanoparticles than a smaller central nanoparticle. Similarly, using smaller peripheral nanoparticles allows more nanoparticles to assemble onto the central nanoparticle.

Nanoparticles with different shapes have different physiochemical properties. Thus, nanoparticle shapes such as nanoplates, nanostars, nanobowls, etc., have unique optical and/or electrical properties that are significantly different from nanospheres or nanoflowers. For example, some embodiments of the disclosed nucleic acid-shaped nanoparticles may have promising applications in SERS (Surface Enhanced Raman Spectroscopy) based biosensing. Raman spectroscopy is a useful technique that detects and identifies molecules based on their vibrational energy levels and corresponding Raman fingerprints. However, Raman scattering from the molecules themselves without enhancement is very weak. Colloidal Au nanospheres have been used to increase the scattering efficiencies of Raman-active molecules by as much as 10¹⁴-10¹⁵-fold.⁴⁴ Compared to these nanospheres, nucleic acid-shaped nanoparticles may be a better candidate for fabricating SERS-active tags for a number of reasons: (i) the tips of the nanoscale bumps and the nanocavities are likely to have strong localized near-field enhancement effects^{36,37}; (ii) embodiments of the disclosed DNA-shaped nanoparticles have a larger total surface area than a nanosphere with a similar diameter; and/or (iii) some embodiments of the DNA-shaped nanoparticles are expected to have surface plasmon resonance peaks that are nearer to the excitation wavelength, which will provide stronger enhancement effects.

Embodiments of the disclosed nucleic acid-shaped nanoparticles also may have an improved performance in imaging and drug delivery in comparison with nanospheres. Furthermore, these nanoparticles with different light scattering properties may be used collectively for multiplex sensing or imaging by encoding each target with a different type of nanomaterial. For example, a nanoplate may be functionalized (e.g., with an antibody or an oligonucleotide probe) to couple to one target, while a nanostar may be functionalized to couple to a different target.

### IV. EXAMPLES

### Chemicals and Materials

A10 oligodeoxyribonucleotides used herein were purchased from Integrated DNA Technologies Inc. (Coralville, Iowa). Solutions of 20-nm and 5-nm gold nanospheres (AuNSs) were purchased from Ted Pella (Redding, Calif.) and purified using a centrifuge before use. Hydrogen tetrachloroaurate(III) hydrate (HAuCl₄.3H₂O, 99.999%; Sigma-Aldrich), hydroxylamine hydrochloride (NH₂OH.HCl, 99.9999%; Sigma-Aldrich), sodium hydroxide (NaOH, 98%; Sigma-Aldrich), adenosine 5′-monophosphate sodium salt (AMP, 99%; Sigma-Aldrich), thymidine 5′-monophosphate sodium salt (TMP, 99%; Sigma-Aldrich), cytidine 5′-monophosphate sodium salt (CMP, 99%; Sigma-Aldrich), guanosine 5′-monophosphate sodium salt (GMP, 99%; Sigma-Aldrich), cetyltrimethlyammonium bromide (CTABr); sodium iodide (99%), L-ascorbic acid (99%); sodium borohydride (NaBH4, 99.99%), tris(2-carboxyethyl)phosphine hydrochloride (TCEP, C₉H₁₅O₆P.HCl; Sigma-Aldrich), 2-mercaptoethanol (ME, 98%; Sigma-Aldrich) and mPEG thiol (CH₂O—(CH₂CH₂O)₆—CH₂CH₂SH, Mw=356.5; Polypure) were used without further purification.

### Synthesis and Purification of Gold Nanoprisms

Gold nanoprisms were prepared by following previously published literature.⁶² The nanoprism synthesis included the following four steps. Step A: 2.733 g of CTABr was added in 150 mL Millipore® water, followed by addition of NaI to reach a concentration of 50 μM. The solution was then heated to dissolve the CTABr. Step B: 5 nm gold nanoparticle seeds were synthesized by reducing 1 mL of 10 mM HAuCl₄ with 1 mL of 100 mM NaBH₄, in the presence of 1 mL of 10 mM sodium citrate and 36 mL of Millipore® water. The gold nanoseeds were aged for at least 2 hours to hydrolyze the unreacted NaNH₄. Step C: Three growth solutions were prepared for nanoprism growth. Solutions 1 and 2 contained 0.25 mL of 10 mM HAuCl₄, 0.05 mL of 100 mM NaOH, 0.05 mL of 100 mM ascorbic acid, and 9 mL of the prepared CTABr solution. Solution 3 contained 2.5 mL of 10 mM HAuCl₄, 0.50 mL of 100 mM NaOH, 0.50 mL of 100 mM ascorbic acid, and 90 mL of the prepared CTABr solution. Step D: For nanoprism synthesis, 1 mL of 5 nm gold nanoparticle solution was added to Solution 1, and then 1 mL of this solution was quickly added to Solution 2. After shaking, Solution 2 in its entirety was added to Solution 3, and the reaction was allowed to progress for at least for at least 30 minutes. The as-prepared nanoprism solution was stored in a glass flask overnight to allow the nanoprisms to settle. Afterwards, the supernatant was removed, and the gold nanoprisms were redispersed in Millipore® water.

### Characterization Methods

The morphology of the gold nanoparticles as well as the nano-assemblies was analyzed using a JEOL 2010LaB6 transmission electron microscope (TEM) operated at 200 kV. Samples were prepared by putting a drop of a nanoparticle solution onto a carbon-coated copper TEM grid (Ted pella). In some examples, morphology was analyzed using a Hitachi 54800 scanning electron microscope (SEM).

Absorbance of the nanoparticle solutions was characterized using UV-Vis-NIR spectrophotometry (Hewlett-Packard 8453 or Cary 5000).

Darkfield light-scattering images were acquired using a Zeiss Axiovert 200M inverted microscope coupled with a CCD digital camera. The individual nanoparticles on a glass coverslip were imaged using an EC Epiplan 50×HD objective (NA=0.7), and the Chinese hamster ovary (CHO) cells were imaged with a Plan-Neofluar 10× objective (NA=0.3). Prior to acquisition, the digital camera was white-balanced using Zeiss Axiovision software so that colors observed in the digital images represented the true color of the scattered light.

Z-stacks of fluorescence images of the cells were acquired using Andor Technology Revolution System Spinning Disk Confocal Microscope at 100× objective (oil immersion, excitation wavelength 488 nm). The collected z-stacks of images were then deconvoluted and assembled into a 3D image using Autoquant X software and Imaris software.

### Example 1

### Nanoparticle Synthesis and Characterization

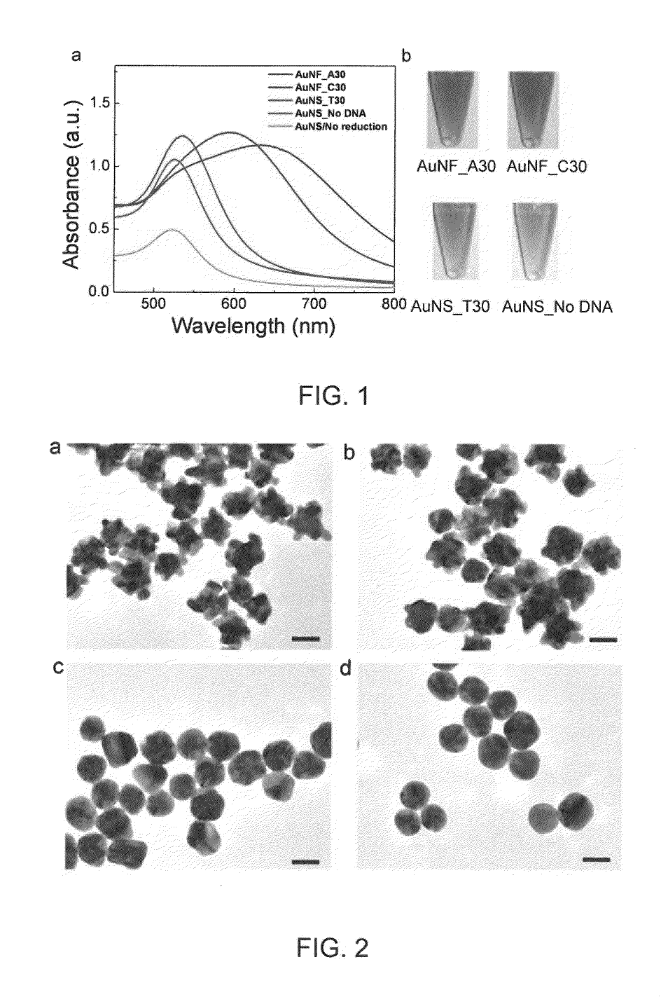

The concentration of purified 20-nm citrate-coated gold nanospheres (AuNSs) was calculated based on the Beer-Lambert law (extinction coefficient of 20-nm AuNS at 520 nm is 9.406*10⁸ M⁻¹cm⁻¹) and then adjusted to 0.5 nM and resuspended in pure water. A 300 μL aliquot of 0.5 nM 20-nm AuNS solution was first incubated with 1 μM of DNA (poly A30, poly C30 or poly T30) for 15 min to let DNA adsorb onto the AuNS surface. This step was followed by addition of 15 μL of 400 mM NH₂OH (adjusted to pH 5 with NaOH) to produce a final concentration of 20 mM NH₂OH. Three types of 30-mer DNAs consisting of poly A, poly C, or poly T (designated as A30, C30, and T30, respectively) were used. After vortexing, 2.1 μL 1% (wt/wt) HAuCl₄ was introduced to AuNS mixture solution (final concentration of HAuCl₄ was 167 μM), and the mixture was rigorously vortexed to facilitate the reduction. A color change was observed in seconds. The mixture solution was constantly vortexed for another 15 min until the reaction was complete. Based on the DNA sequences used and their shape, the synthesized gold nanoparticles were called AuNF_A30, AuNF_C30 or AuNS_T30 respectively. Surprisingly, nanoparticle solutions synthesized in the presence of A30 or C30 were blue colored, while the nanoparticle solution synthesized with T30 was red colored (FIG. 1b). The resultant solutions were stable for days without showing any nanoparticle aggregation or color change.

To determine the morphology of the nanoparticles prepared with different DNA sequences, transmission electron microscopy (TEM) was employed to investigate each of the resulted nanoparticle solutions. Surprisingly, those particles synthesized with A30 or C30 were flower shaped (designated as AuNF_A30 and AuNF_C30) (FIGS. 2a, 2b), while particles synthesized with T30 were spherical (AuNP_T30, FIG. 2c). The flower-shaped gold nanoparticles had a broad surface plasmon absorbance that peaked at 600 nm (for AuNF_C30) or 630 nm (for AuNF_A30) (FIG. 1a), which is consistent with the absorbance of gold nanoflowers prepared by other reported methods.³⁶

Poly G30 was not tested due to synthetic difficulties caused by the formation of a guanine tetraplex structure.³⁷ Instead, a shorter DNA consisting of 10-mer poly G was tested, and the resulting nanoparticles were nearly spherical (FIG. 3). In contrast, only spherical nanoparticles were formed in the absence of DNA (FIG. 2d) or in the presence of salt only (FIGS. 4a and 4b).

No metal nanoparticles were formed upon mixing DNA, NH₂OH and HAuCl₄ together, without the addition of AuNS as seeds. These results demonstrated that the DNA mediates the morphology of the gold nanoparticles, and the nanoparticle shape is sequence dependent.

To understand the DNA sequence-dependent nanoparticle formation and to determine the stability of DNA-adsorbed AuNs, the adsorption step of single-stranded DNA (ssDNA) on AuNS was investigated. Unmodified ssDNA is able to adsorb onto AuNS, and enhances the electrostatic repulsion between AuNSs, thereby reducing or preventing salt-induced aggregation.³⁸ First, 100 μL of 1 nM, 20 nm AuNS solutions were incubated with 1 μM DNA (either poly A30, poly C30, or poly T30, respectively). After 15 min incubation, 0.1 M NaCl was introduced to each of the solutions. UV-vis spectroscopy was used to record the absorbance of each solution before and after the addition of NaCl.

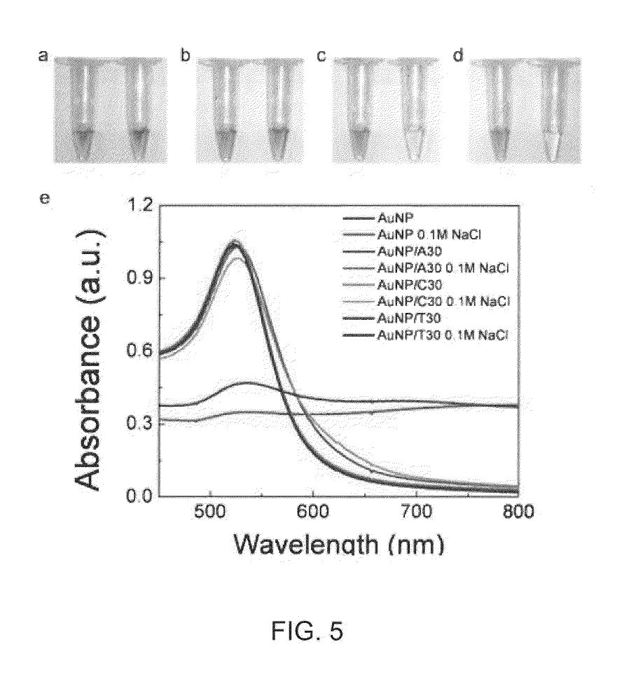

As shown in FIGS. 5a-5e, aggregation of AuNS happened immediately when the T30 DNA sequence was used for incubation with the AuNS, while AuNS incubated with A30 or C30 sequences remained stable. Since the stability of the AuNS at the same salt concentration is determined by the number of DNA adsorbed on its surface,³⁹ it was concluded that many fewer T30 molecules were adsorbed onto the AuNS surface compared to A30 or C30, which is consistent with the lower binding affinity of T30 towards the gold nanoparticle surface. This result explains the differences in shaping the gold nanoparticle by the T30 sequence in comparison with A30 or C30.

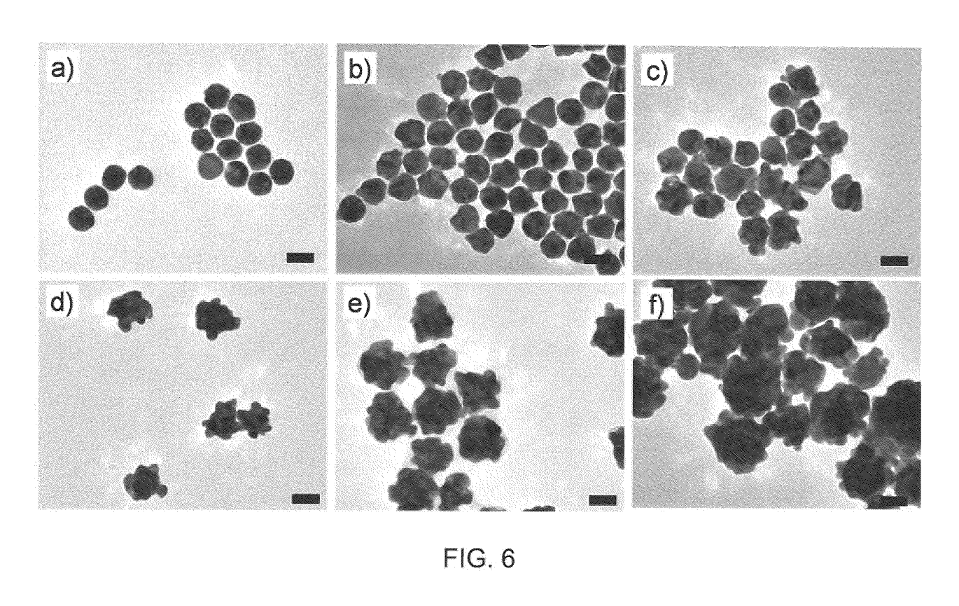

To further evaluate the mechanism of shape control process of the flower-shaped nanoparticle directed by DNA, varying amounts of HAuCl₄ were added to A30, which was incubated with AuNS and 20 mM NH₂OH to initiate the reduction. Since NH₂OH was in large excess, it was expected that the HAuCl₄ would be completely reduced to gold metal in the presence of AuNS seeds.³⁵ As shown in FIGS. 6a-6f, with the addition of increasing amount of HAuCl₄, the resultant nanoparticle evolved from sphere shape to a bud sphere and then into the flower-like shape. Upon further increase of the HAuC14 amount, the flower shaped nanoparticle would grow even bigger.

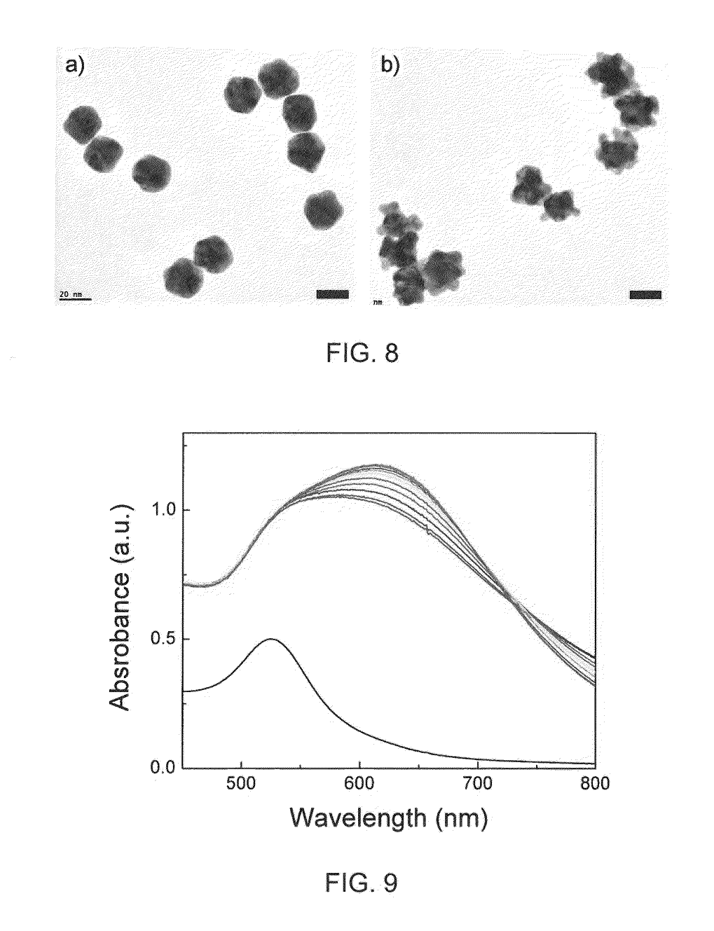

In order to investigate how the nanoparticle morphology was affected by the number of DNA oligomers adsorbed on AuNS, varying amounts of A30 were incubated with AuNS and followed by reduction of equal amounts of HAuCl₄. FIGS. 7a-7f show that the nanoparticle shape changed from spherical to flower-like with increasing numbers of DNA oligomers adsorbed on AuNS, while the size of the gold nanoparticle remained the same. From the above observations, it was determined that DNA of chain-like structure was able to direct the deposition of the reduced gold metal on the AuNS and guide the nanoparticle growth from a spherical into a flower-like shape. This conclusion was further supported by the control experiments, which showed that when the single deoxynucleotide, adenosine monophosphate (AMP) was incubated with AuNS instead of a DNA chain, the nanoparticles obtained were nearly spherical, while a random 30-mer DNA sequence of mixed A, T, G, C caused the formation of flower-shaped nanoparticles (FIGS. 8a-8b).

To further probe this DNA mediated AuNF growing process, the absorbance of AuNF growth solution was monitored using UV-visible spectroscopy. As shown in FIG. 9, after initiation of the reaction for 3 seconds, the intensity of the nanoparticle absorbance increased significantly, and the peak of the AuNSs at 520 nm broadened and red-shifted. With growth of the AuNS, a new absorbance peak at 630 nm from the resultant AuNFs appeared, and the reaction completed in about 15 minutes.

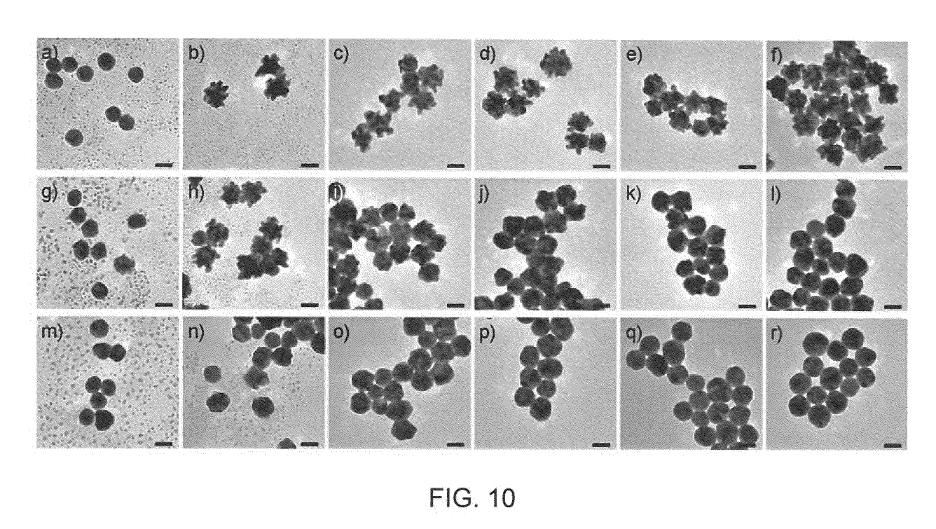

This time-dependent AuNF growth process was further studied using TEM by stopping the reaction at the early stages of NP growth with excess mercaptopropionic acid (MPA). MPA has been shown to quench the NP growth effectively by forming the less reactive Au(I)-MPA complex with gold ion.⁴⁰ As shown in FIGS. 10a-10r, both the 20-nm AuNSs and 1-3 nm small nanoparticles (SNPs) could be observed after initiation of the reaction at 0.5 second.

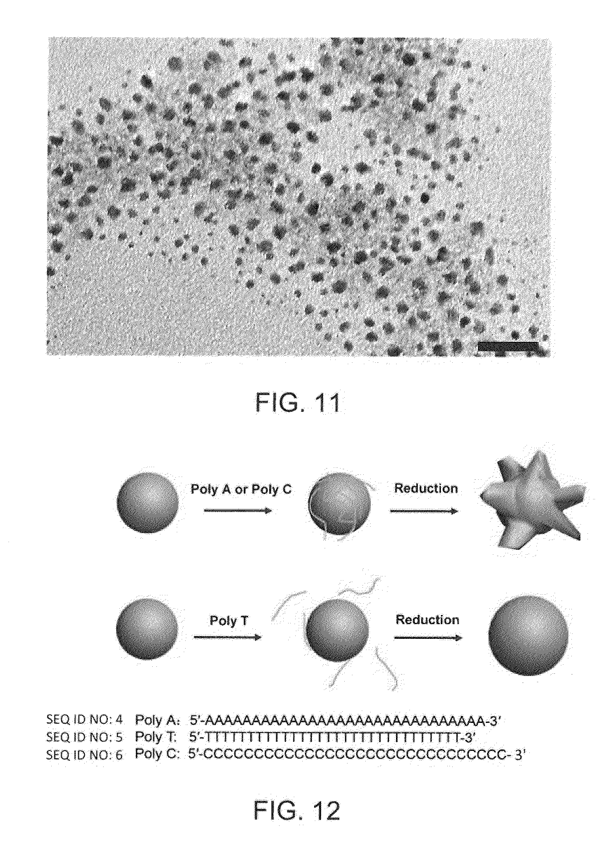

A further control experiment showed that formation of the SNPs could be due to the conversion of Au(I)-MPA complexes into metal particles on the TEM grid upon electron-beam irradiation during TEM imaging (FIG. 11). Flower-like nanoparticle intermediates were observed after 2 seconds of reaction in both A30 and T30 mediated syntheses. Interestingly, the flower-like intermediates prepared with T30 grew further into nanospheres within 30 s while the intermediates prepared with A30 maintained their flower-like structure and stable AuNFs were produced. In the absence of DNA, the AuNSs grew into bigger nanospheres and no flower-like intermediate was observed. These results suggest that DNA adsorbed on the AuNS surface acts as a template to mediate the formation of flower-like gold nanoparticles. The formation of the AuNF results from either selective deposition of the reduced gold metal on AuNS templated by surface-bound DNA or from uneven growth of the AuNS due to the binding of DNA to the surface.

As depicted in FIG. 12, due to the strong binding affinity of poly A (SEQ ID NO: 4) or poly C (SEQ ID NO: 6) to AuNS, a number of A30 or C30 bind tightly to AuNS and induce the inhomogeneous growth of AuNS, producing the flower-like nanoparticles. In contrast, fewer poly T molecules bind weakly and loosely to AuNS. The weakly bound poly T molecules produce the flower-like intermediates at a very initial stage. However, they are not able to stabilize the flower-like structures, and the spherical particles are eventually formed.

### Example 2

### Determination of the Number and Stability of Thiolated and Unmodified Oligonucleotides on Gold Nanoflowers

### Preparation of Thiolated DNA-Gold Nanoflowers

Functionalization of thiolated DNA (HS-A30 or HS-T30) on 5-nm gold nanospheres was carried out by following a published protocol⁵⁵ with slight alterations. Briefly, 9 μL of 1 mM thiolated DNA was first mixed with 1.5 μL of 10 mM TCEP (tris(2-carboxyethyl)phosphine) solution and 1 μL of 500 mM acetate buffer (pH 5.2) to activate the thiolated DNA. After a 30-minute reaction, the mixture was transferred into 3 mL of 5-nm AuNS solution (82 nM, in pure water) followed by addition of 10 mM Tris-HCl buffer (Tris=2-amino-2-hydroxymethyl-1,3-propanediol, pH 8.2). The nanoparticle solution was incubated overnight, and the NaCl concentration was then increased to 0.1 M. The functionalized 5-nm AuNS solutions (designated as AuNS₅ₙₘ_{_{—}}S_A30 or AuNS₅ₙₘ_{_{—}}S_T30) were incubated for another 12 h before usage. To purify the nanospheres from the unreacted DNA, a Microcon® centrifugal filter (Ultracel YM-100, MWCO=100K; Millipore, Billerica, Mass.) was used by following the instructions from the manufacturer.

### Preparation of Unmodified DNA Gold Nanoflowers

Fluorophore (FAM) labeled poly A30 was used for AuNF synthesis. The AuNFs were synthesized by incubating 1 μM of Fluorophore (FAM) labeled poly A30 (FAM-A30) with 300 μL of 0.5 nM 20 nm AuNS solution for 15 min. 15 μL of 400 mM NH2OH (pH 5) and 2.1 μL 1% (wt/wt) HAuCl4 were added to the nanoparticle solution to initiate the AuNF formation (three samples were prepared separately). Meanwhile, 300 μL 1 μM FAM-A30 solutions were prepared with the addition of 15 μL of 400 mM NH₂OH (pH 5) and 2.1 μL pure water and these solutions were used as control solutions. After AuNF synthesis, the supernatants were collected by removing the nanoparticles with centrifugation. The oligonucleotide concentrations in both the collected supernatants and the control solutions were quantified and compared by using UV absorbances at 260 nm. The DNA concentration in the supernatants was 825.6 nM, so the DNA attached to the AuNFs during synthesis were 174.4 nM. Dividing this number by the AuNS concentration (0.5 nM), it was estimated that the average number of attached oligonucleotides on each AuNF was ˜349.

### Stability of Attached Oligonucleotides.

To probe the stability of the DNA attached to AuNFs, the number of oligonucleotides on AuNFs after treatment with mercaptoethanol was quantified using a fluorescence-based method.⁵⁶ The AuNF solutions (0.5 nM) were treated with mercaptoethanol (ME) to a final concentration of 14 mM overnight. The solutions containing the displaced oligonucleotides were separated from AuNFs by centrifugation. Each supernatant (100 μL) was added to 400 μL 62.5 mM phosphate buffer (pH 7.2). The pH and ionic strength of the sample and calibration standard solutions were kept the same for all measurements due to the sensitivity of the fluorescent properties of FAM to these conditions. The fluorescence maximums (520 nm) were measured and then converted to molar concentrations of the FAM labeled oligonucleotides by using a standard linear calibration curve. Standard curves were carried out with known concentrations of fluorophore-labeled oligonucleotides under same buffer pH, salt, and mercaptoethanol concentrations.

The average number of displaced oligonucleotides for each AuNF was obtained by dividing the calculated oligonucleotide molar concentration by the original AuNF concentration. The results demonstrated only ˜110 strands were displaced by mercaptoethanol (ME), and the majority (˜240 strands) was still bound to the AuNF after the treatment. Thiol-gold chemistry is the most used method to conjugate DNA to gold surface. Under the same ME (14 mM) treatment, however, all of the thiolated DNA oligonucleotides were displaced by ME from the gold surface.⁴²

### Example 3

### Melting Point Determination of DNA-Functionalized Gold Nanoflowers

Considering the remarkably high binding affinity of DNA to the AuNFs (higher than thiol-gold binding), it was hypothesized that the DNA in situ attached to AuNFs during reduction could be partially buried in the AuNFs. To test this hypothesis and also the functionality of the DNA on the AuNFs, experiments were performed to test the melting point of the DNA in-situ attached on the AuNFs.

AuNFs were first treated with thiolated PEG (polyethylene glycol, 6 μM) molecules overnight to displace any weakly bound DNA on AuNF surfaces.⁴³ Purified AuNF_A30 (2 nM) was hybridized with fluorophore (FAM) labeled Poly T30 (FAM-T30) (1 μM) in a buffer solution containing 10 mM HEPES buffer (pH 7.1) and 50 mM NaCl. The mixture solution was heated up to 65° C. and cooled down to room temperature in about two hours. The unhybridized fluorophore strands were removed by centrifugation, and the AuNFs (2 nM) were redispersed in the same buffer solution.

A fluorimeter (FluoroMax-P; Horiba Jobin Yvon, Edison, N.J.) coupled with a temperature controller was used to obtain the melting curve of the DNA hybridization on AuNFs. Since a gold nanoparticle can effectively quench the fluorescence from its surrounding fluorophores, the release of the fluorophore labeled DNA from AuNFs due to DNA melting will result in a fluorescence increase of the nanoparticle solution. The sample was kept at target temperatures for 72 seconds after the temperature was reached to ensure that the sample was at the stated temperature during data collection at each temperature. As a comparison, free A30 labeled with an organic quencher (Blank Hole Quencher-1, 200 nM)) was hybridized with FAM-T30 (200 nM) in the same buffer under identical conditions, and its melting curve was collected as well.

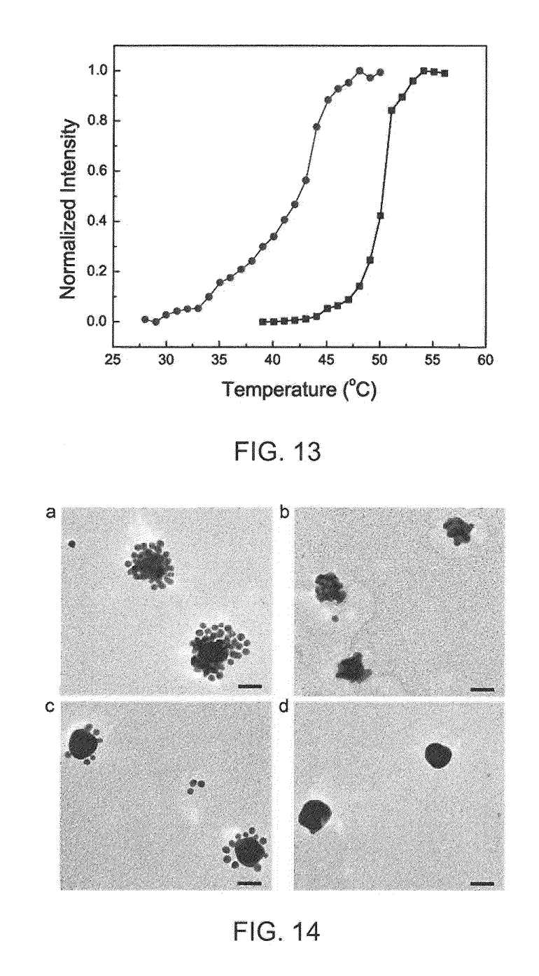

As shown in FIG. 13, the melting temperature of the DNA in situ attached to AuNFs (around 42° C.) was significantly lower than the free DNA (around 50° C.). This result indicated that a small segment of DNA might be buried in the AuNFs during the nanoparticle growth, while the majority part of DNA exposed outside was still functional for DNA hybridization.

### Example 4

### Nano-Assembly of DNA-Functionalized Gold Nanoparticles

The synthesized AuNF_A30 solution was first purified by centrifugation (9000×g, 5 min.) twice and then redispersed in water. The AuNF_A30 particles were then treated with 6 μM mPEG thiol for 2 hours and purified. After purification, AuNF_A30 (0.5 nM) was mixed with purified AuNS (50 nM) modified with thiolated complementary DNA (AuNS₅ₙₘ_{_{—}}S_A30 or AuNS₅ₙₘ_{_{—}}S_T30 respectively) in the presence of 10 mM phosphate buffer (pH 8) and 0.1 M NaCl. The mixture solution was incubated overnight to allow nano-assembly. The same procedure was used to assemble AuNS_T30 with AuNS₅ₙₘ_{_{—}}S_A30 or AuNS₅ₙₘ_{_{—}}S_T30. After incubation, the nanoparticle mixture solution was centrifuged at (9000×g, 2 min.) to remove free 5-nm gold nanoparticles in the supernatant, and the pellet was redispersed in buffer solution for TEM sample preparation.

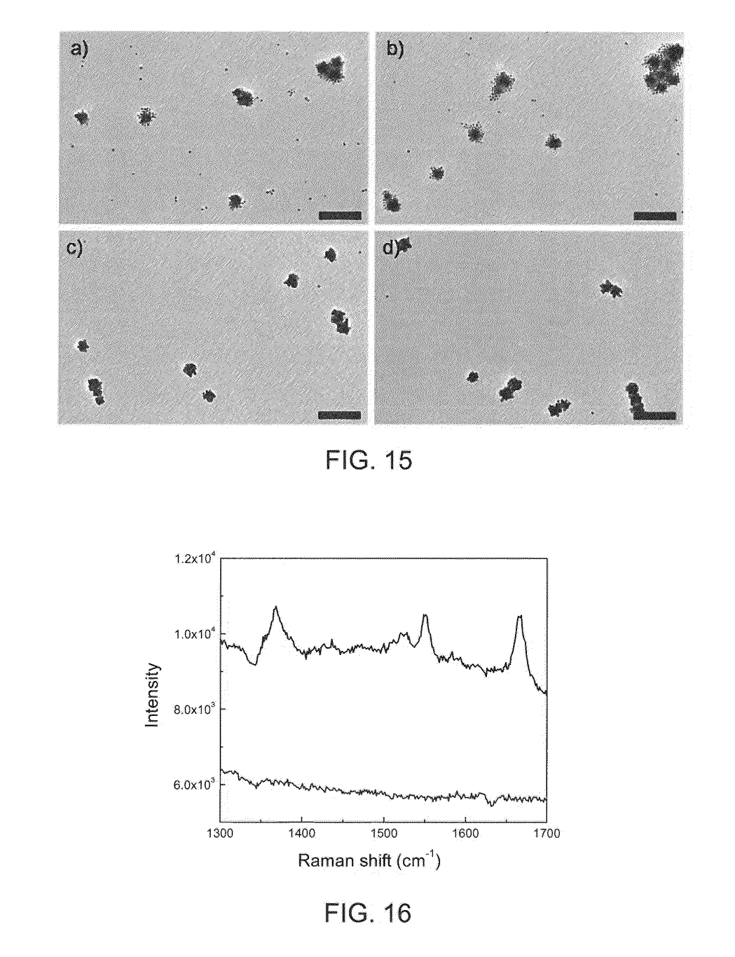

TEM was then employed to assess the assembly of the nanoparticles. As shown in FIG. 14a, AuNF_A30 was surrounded by a number of AuNS5 nm_S_T30, forming the satellite structure. As a comparison, when 5-nm AuNS functionalized with non-complementary DNA A30 (AuNS5 nm_S_A30) were used to incubate with AuNF_A30, no assembly was observed (FIG. 14b). Additional large-area TEM images containing multiple satellite assembled nanostructures are shown in FIGS. 15a-15d. These results further confirmed that the DNA molecules were not only densely functionalized to AuNFs in a large number, but also retained their molecule recognition properties. Interestingly, when AuNS_T30 were incubated with AuNS5 nm_S_A30 under similar conditions, only a few 5-nm particles were assembled on AuNS_T30, while little assembly was observed with non-complementary AuNS5 nm_S_T30 (FIGS. 14c, 14d). This observation indicates that fewer numbers of T30 oligonucleotides were attached during synthesis, consistent with the fact that fewer T30 oligonucleotides were adsorbed on AuNS compared to A30 or C30.

### Example 5

### Surface Enhanced Raman Spectroscopy of DNA-Functionalized Gold Nanoflowers

SERS enhancement from DNA functionalized AuNFs was compared with AuNSs. Raman tag labeled DNA (Trama-A30) was used to grow AuNFs and then the Raman signal was collected. As shown in FIG. 16, under the same conditions (excitation (603 nm), nanoparticle concentration (0.5 nM), etc.), the Raman signal from the Raman tag with the AuNFs was clearly observed while the signal from the Raman tag with AuNS was too low to distinguish. These results indicated that AuNFs provide a much stronger SERS effect over the AuNS.

### Example 6

### Cellular Uptake of Gold Nanoflowers

AuNFs were synthesized with 1 μM of fluorophore (FAM) labeled poly A30 (FAM-A30) by following the procedure in Example 2. The AuNFs were purified by centrifugation.

CHO (Chinese hamster ovary) cells were cultured in Dulbecco's modified eagle medium (DMEM; Cell Media Facility, University of Illinois at Urbana-Champaign, Urbana, Ill.) supplemented with 10% fetal bovine serum (FBS), penicillin (50 U/ml), and streptomycin (50 μg/ml), at 37° C. in a humidified atmosphere of 5% CO2. Cells were seeded at a density of 1×10⁵ cells/cm² on 4 well Lab-Tek chambered #1 Borosilicate coverglass system (Fisher Scientific), and the cells were grown for 24 hours before treatment with nanoparticles. After 18 hours, the cells were washed with 1×PBS buffer and fresh media was added.

To investigate the cell uptake of the AuNFs, nanoparticles (0.5 nM or 1 nM) synthesized with fluorophore (FAM) labeled A30 were added to the cells and incubated for 18 hours. Excess AuNFs were removed by washing the cells with 1×PBS five times prior to imaging.

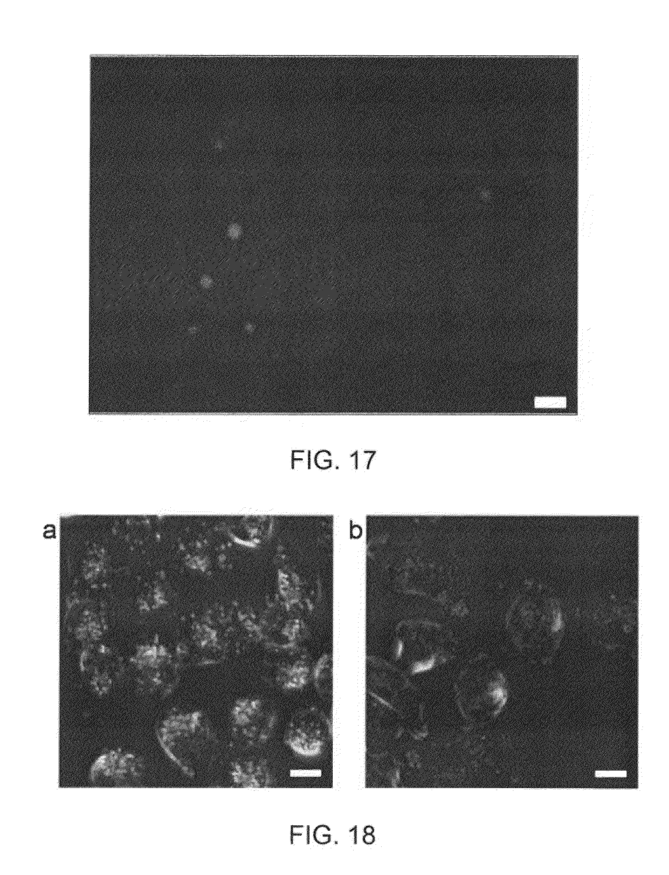

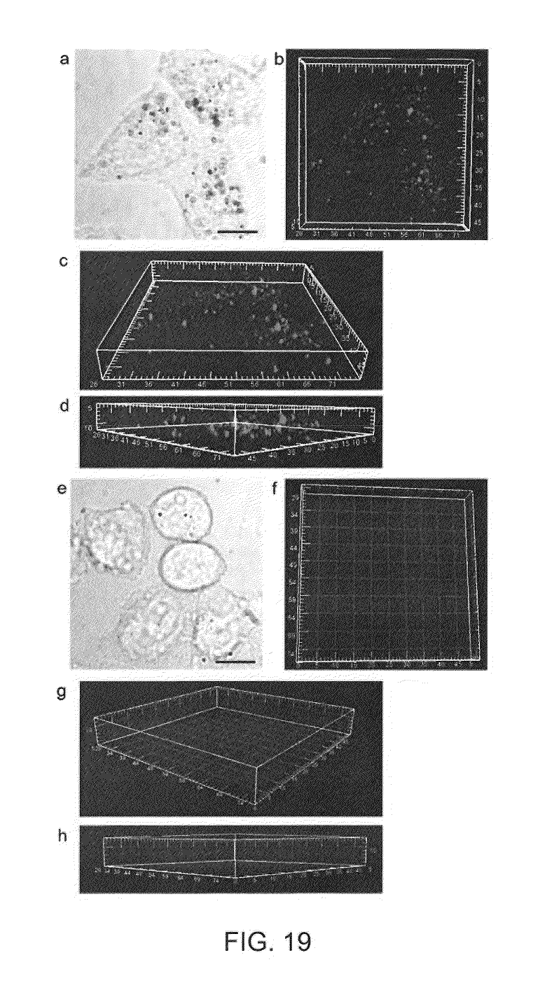

Dark-field light-scattering images were taken to visualize the AuNF uptake by the cells.⁴⁷ The light scattering property of the AuNFs was first investigated using a dark-field microscope coupled to a CCD digital camera. The digital camera was white-balanced so that the observed colors represented the true color of the scattered light. The AuNFs showed bright orange color in the dark field image (FIG. 17). As shown in FIG. 18a, the orange dots representing the AuNFs were observed in the intracellular region of the cells while the untreated control cells appeared dim yellow to green color due to the intrinsic cellular scattering (FIG. 18b). This nanoparticle cellular uptake was further confirmed by the 3-D reconstructed confocal microscope images of the AuNF treated cells, showing that the AuNFs were distributed inside the cells (FIGS. 19a-19h). The results demonstrated that AuNFs entered into cells during the incubation. This ability of the AuNF to be taken up by the cell could be due to the high DNA loading on the AuNF surface⁴⁸ and/or the shape effect.⁴⁹ The cellular uptake ability and light scattering property of AuNFs make them useful as nanocarriers for drug or gene delivery and contrast agents for intracellular imaging.

### Example 7

### Synthesis of Non-Spherical Nanoparticles

Gold nanoprisms were synthesized in the presence of surfactants and iodine by following a previously reported method.⁵⁰ After removing the free surfactant with centrifugation, these purified nanoseeds were incubated with DNA of different sequences (A30, T30, C30) respectively for 15 minutes. NH₂OH and HAuCl₄ were then added to the nanoparticle solution to initiate the particle growth.

The morphologies of the prepared nanoparticles were studied using scanning electron microscopy (SEM). Surprisingly, the nanoprisms incubated with A30 or C30 grew into thicker round nanoplates, while nanoprisms incubated with T30 grew into 2-D six-angled nanostars (FIGS. 20a-20c). Nanoprisms incubated with G10 also produced 2-D multiple angled nanostars were produced (FIG. 20d). These results demonstrated that DNA of different sequences can direct the growth of the nanoprism into different shapes, and that each sequence encodes the formation of nanoparticles with certain shapes.

Nanoparticle growth was also tested using gold nanorods as seeds. Remarkably, the nanorods (FIG. 21a) were converted into dogbone-like nanoparticles in the presence of A30 after growth (FIG. 21b), while the nanorods were converted into peanut-like nanoparticles in the presence of T30 (FIG. 21c).

These results indicate that embodiments of the DNA-mediated shape-control method can be readily adapted to synthesize other non-spherical nanoparticles. Thus, this method can be used as a general methodology to control growth of metal nanoparticles, permitting production of a series of novel nanoparticles with different shapes and unique properties.

### Example 8

### Nanoflower Size and Quality Control

Nanoflower size can be precisely controlled by controlling the growth conditions for nanoflowers, e.g., by varying the amount of gold available and/or by varying the nanoseed size.

In one example, nanoflowers were synthesized using 300 μL of a 0.5 nM solution of 13-nm gold nanoseeds (synthesized according to available protocols) with increasing amounts of a 1% w/v solution of HAuCl₄, and the resulting nanoflowers were analyzed by TEM. The nanoseeds were incubated with an AS1411 aptamer (1 μM; SEQ ID NO: 2) or a randomized control construct (1 μM; SEQ ID NO: 1) prior to gold salt reduction. The protocol described above in Example 1 was followed during synthesis.

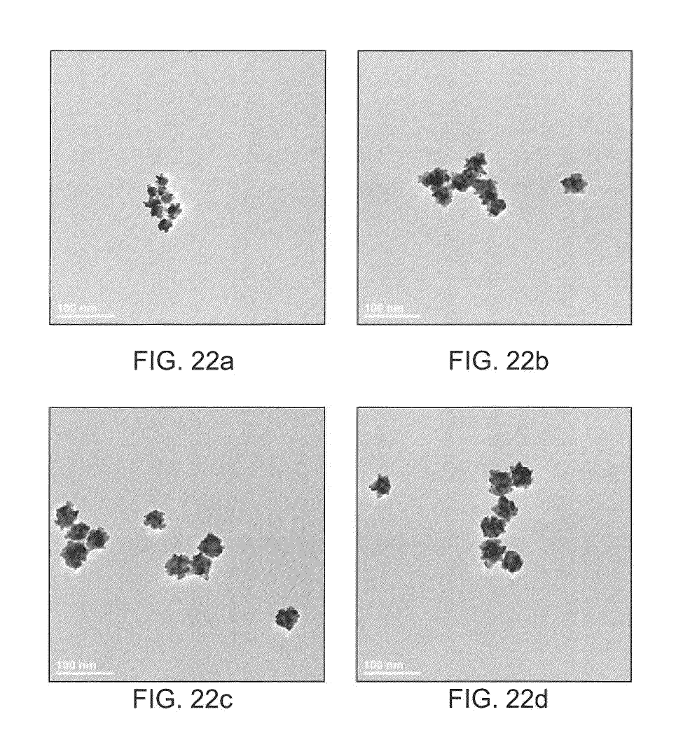

As shown in FIGS. 22a-22d, increasing gold salt concentration under identical conditions leads to increasing nanoflower size with good uniformity. Using additional gold resulted in non-uniform structures. FIGS. 22a-22d are TEM images of nanoflowers synthesized with the AS1411 aptamer under the following conditions:

TABLE 2

| | 0.5 nM, 13 nm seed | NH₂OH (400 mM) | 1% w/v HAuCl₄ |
|---|---|---|---|
| FIG. 22a | 300 μL | 15 μL | 0.7 μL |
| FIG. 22b | 300 μL | 15 μL | 0.9 μL |
| FIG. 22c | 300 μL | 15 μL | 1.3 μL |
| FIG. 22d | 300 μL | 15 μL | 1.5 μL |

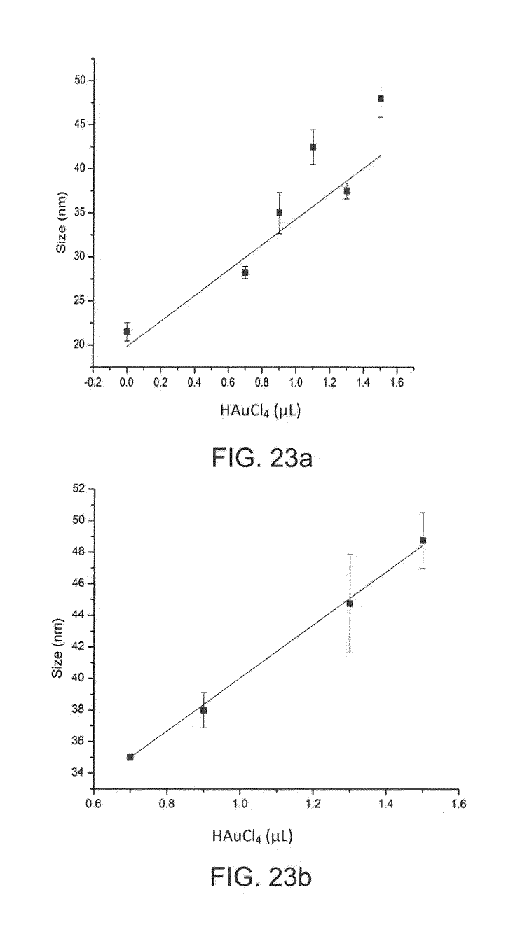

The relationship between gold salt concentration and nanoflower size was determined to be linear (FIGS. 23a-23b). The nanoflowers in FIG. 23a were synthesized with the randomized DNA construct, and the nanoflowers in FIG. 23b were synthesized with the AS1411 aptamer.

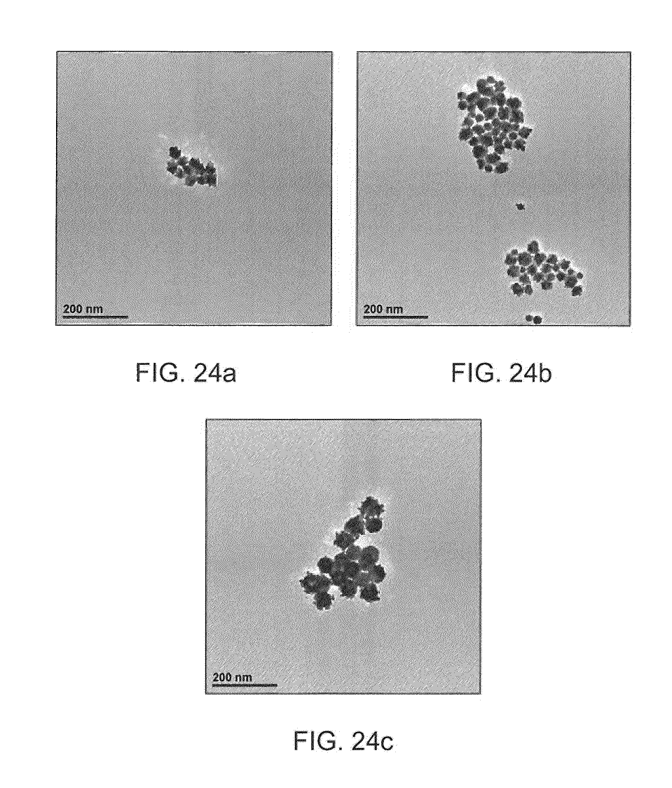

In another example, nanoflower size was controlled by varying the size of the nanoseed. Nanoflowers were synthesized using 1 μM AS1411 aptamer and 1% w/v HAuCl₄ with 15-nm, 30-nm, and 50-nm gold nanoparticles as nanoseeds. FIGS. 24a-24c are TEM images of the nanoflowers grown from 15-nm, 30-nm, and 50-nm gold nanoparticle seeds synthesized with the AS1411 aptamer under the conditions shown in Table 3. The protocol described above in Example 1 was followed during synthesis. As seen in FIGS. 24a-24c, the nanoflower size increased with increasing nanoseed size.

TABLE 3

| | 200 μL AuNP | NH₂OH (400 mM) | 1% w/v HAuCl₄ |
|---|---|---|---|
| FIG. 24a | 15 nm, 0.5 nM | 15 μL | 3 μL |
| FIG. 24b | 30 nm, 0.31 nM | 15 μL | 3 μL |
| FIG. 24c | 50 nm, 0.06 nM | 15 μL | 4 μL |

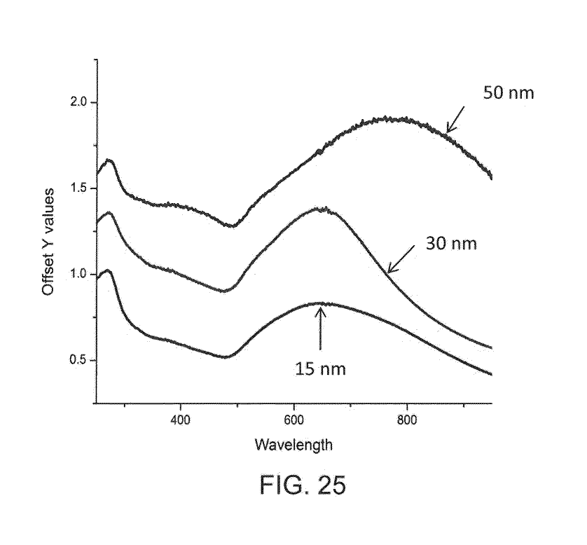

The nanoflower structure is ideally suited for photothermal applications, and embodiments of the synthesized nanoflowers can be tuned to absorb strongly within the near-IR window (i.e., from 700 nm to 900 nm). As shown in FIG. 25, the nanoflowers grown from 50-nm gold nanoparticle seeds are candidates for photothermal applications with an absorption peak at 800 nm.

### Example 9

### Cancer-Selective Targeted Uptake In Vitro

Two types of nanoflowers were synthesized. The first nanoflower included the AS1411 apatmer (SEQ ID NO: 2); the second construct was identical except the aptamer sequence was randomized (SEQ ID NO: 1). The DNA sequences are shown in Table 4 below. Both types of nanoflowers were grown from 15 nm gold seeds and incubated with MCF-7 cells (human breast cancer cells). Nanoflowers were synthesized following the protocol described above in Example 1.

Cells were incubated and grown according to standard procedures and plated on glass cover slips inside a 6-well plate (−100,000 cells per well). Cells were incubated for 12 hours in cell medium (10% FBS) and washed with PBS buffer. After washing, the cells were incubated with 100 μL of nanoflower solution (10 nM suspended in deionized water) diluted with 900 μL of Opti-MEM for 2 hours at 37° C. and 5% CO₂. After incubation, the cells were washed 3× with PBS to remove excess nanoflowers, and the glass slides were processed for imaging under fluorescence microscope and dark-field optical microscope.

TABLE 4

| | | |
|---|---|---|
| Control- | SEQ ID | 5′-/56-FAM/TTG GTA GTA GTG ATT GTA ATG GTA GTG |
| DNA | NO: 1 | A TTTTT TTTTT TTTTT CCCCC CCCCC CCCCC CCCCC CCCCC |
| | | CCCCC-3′ |
| Aptamer- | SEQ ID | 5′-/56-FAM/TTG GTG GTG GTG GTT GTG GTG GTG GTG |
| DNA | NO: 2 | G TTTTT TTTTT TTTTT CCCCC CCCCC CCCCC CCCCC CCCCC |
| | | CCCCC-3′ (AS1411 aptamer sequence in bold) |

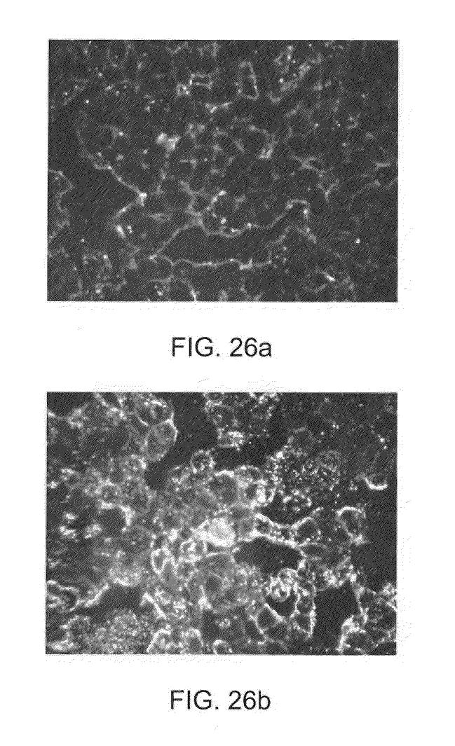

As shown in FIGS. 26a and 26b, nanoflowers functionalized with the AS1411 aptamer (FIG. 26b) exhibited superior binding to the MCF-7 cells compared to nanoflowers comprising control DNA (FIG. 26a).

### Example 10

### Nanoparticle Synthesis

A freshly redispersed gold nanoprism solution was purified by centrifugation twice, and the absorbance of the purified gold nanoprisms was measured by UV-vis spectrometer. The absorbance of the nanoprism solution at 800 nm was adjusted to 0.7 with appropriate dilutions. 100 μL of the diluted gold nanoprism solution was incubated with 2 μM of DNA for 15 min to let DNA adsorb onto the gold nanoseeds. 1 μL of 200 mM NH₂OH (adjusted to pH 5 with NaOH) was then added to the nanoprism solution. After vortexing, 2.5 μL 1% (wt/wt) HAuCl₄ was introduced to the solution to initiate the reduction reaction. A color change (depending on the sequence of the DNA used in the incubation step) was observed in a few minutes, and the reaction was allowed to proceed for at least 15 minutes.

### Example 11

### Nanoparticle Synthesis with Oligomers of a Single Nucleotide

Purified gold nanoprism seeds were incubated with poly A30, poly T30, poly C30, or poly G20 individually (designated as A30, T30, C30, G20, respectively; G20 was tested instead of G30 due to the difficulty in synthesizing DNA longer than 20 G bases), followed by addition of mild reducing agent hydroxylamine (NH₂OH) and gold salt hydrogen tetrachloroaurate(III) (HAuCl₄) to initiate particle growth, as described in Example 10. As controls, nanoprisms also were reacted with HAuCl₄ in the absence of DNA, or reacted with DNA in the absence of HAuCl₄.

The morphologies of the resulting nanoparticles were determined using scanning electron microscopy (SEM). FIG. 28a shows that nanoprisms incubated with A30 resulted in round nanoplates with bumpy surfaces when additional gold was reduced onto the oligomer-functionalized nanoprism. Nanoprisms incubated with C30 also produced round nanoplates; however, the C-30 derived nanoplates had smooth surfaces (FIG. 28c). Nanoprisms incubated with T30 grew into 6-pointed nanostars (FIG. 28b), and nanoprisms incubated with G20 produced hexagonal nanoplates (FIG. 28d). In each case, the nanoparticles were uniformly shaped and mono-dispersed. However, in the absence of DNA, larger than micro-sized gold agglomerations with irregular shapes were formed (FIG. 28e). In the absence of HAuCl₄, minimal shape change was observed (FIG. 28f).

The effect of DNA length was evaluated by varying the DNA length from 5 bases to 30 bases or (5-20 bases for poly G). As shown in FIGS. 29a-29d, when the length of each type of DNA changed from 5 to 30 bases for poly A, poly T, and poly C (FIGS. 29a-29c, respectively), or from 5 to 20 bases for poly G (29d), the resulting nanoparticles had similar shapes regardless of the length, indicating that it is the nucleotide sequence rather than the DNA length that determines the nanoparticle morphology.

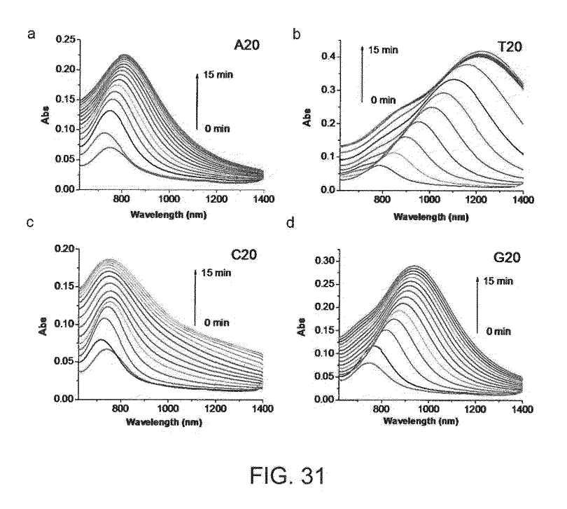

To further support this conclusion, the same procedure was repeated using monomeric dAMP, dTMP, dCMP, or dGMP. As shown in FIGS. 30a-30d, each type of deoxyribonucleotide produced similar-shaped nanoparticles as the corresponding polydeoxyribonucleotides. However, the nanoparticles synthesized with mononucleotides were less stable and tended to aggregate more readily than those synthesized with longer DNA oligomers. Nonetheless, the results demonstrated that DNA of different sequences could direct the growth of the nanoprism into different shapes, and each type of polydeoxyribonucleotides encoded the formation of nanoparticles with certain shapes.

### Example 12

### Kinetics of Nanoparticle Growth Mediated by DNA Molecules

To probe the kinetics of nanoparticle growth process mediated by DNA molecules, the absorbances of nanoparticle solutions growing with polydeoxyribonucleotides were monitored with UV-visible-NIR spectrometer. Nanoparticles were grown in the presence of A20, T20, C20, or G20. Absorbance was monitored for 15 minutes at one-minute time intervals. Gold nanoparticles were synthesized according to the general procedure of Example 10.

For the cases of A20 and C20 (FIGS. 31a, 31c), the absorbance of the nanoprisms at 800 nm blue shifted in the first two minutes and then shifted right back with a peak at 800 nm afterwards. The nanoparticle growth was complete within 10 minutes, and the intensity of the absorbance increased during nanoparticle growth. The absorbance of the sample with G20 also showed an initial blue shift in the first minute of growth, and then red-shifted with a peak at 950 nm (FIG. 31d). In contrast, for the growth solution containing T20, the absorbance increased and the peak at 800 nm red-shifted continuously over the course of particle growth and the final resulted nanoparticle solution showed a broad peak at 1250 nm (FIG. 31b). This difference in absorbance change might indicate different shape evolution pathways for nanoparticle growth from different DNA sequences.

To further study this time-dependent shape evolution, the nanoparticle growth was stopped at different time stages over a 20-minute period by addition of an excess amount of mercaptopropionic acid (MPA). The morphologies of the intermediates were then observed under SEM. As shown in FIG. 32, the nanoprisms with A20 or C20 first grew larger into truncated prisms or round nanoplates in the first three minutes, and then the horizontal growth slowed down while the vertical growth became more evident. While both A20 and C20 produced thicker particles, the A20 induced the formation of round nanoplates with bumpy surfaces, and C20 produced round nanoplates with smooth surfaces. On the other hand, the nanoprisms incubated with T20 grew horizontally first into hexagonal plates in the first two minutes. Each angle of the hexagon then sharpened, and the six-pointed nanostars were well formed after ten minutes of particle growth. In the case of G20, the nanoprisms first evolved into round nanoplates, and then into nanohexagons within ten minutes. These results suggest that DNA can act as a template to mediate the shape evolution of the nanoseeds into different shapes depending on the DNA sequences and/or the reaction time.

### Example 13

### Nanoparticle Synthesis with Di-deoxyribonucleotide Combinations

To explore the possibility to use DNA sequence as a coding system to modulate nanoparticle shape, the growth of nanoparticles using DNA containing two types of deoxyribonucleotides was investigated. Six possible di-deoxyribonucleotide combinations (AT, AC, AG, TC, TG, GC) were tested. The total length of DNA containing two types of deoxyribonucleotide was fixed at 30 nucleotides. The 30 nucleotides were divided into two segments, with each segment containing only one type of deoxyribonucleotide, for example, T5A25 (SEQ ID NO: 20), T10A20 (SEQ ID NO: 21), T15A15 (SEQ ID NO: 22), T20A10 (SEQ ID NO: 23), and T25A5 (SEQ ID NO: 24) for the TA combination. Following the general procedure of Example 10, gold nanoparticles were synthesized from gold nanoprisms in the presence of the di-deoxyribonucleotide oligomers.

As shown in FIG. 33a, nanoparticles grown in the presence of all the above TA combinations gave round nanoplates with rough surfaces, similar to what was observed in the presence of poly A30 alone (FIG. 28a), suggesting that the effect from A was dominating when A and T were combined in one DNA sequence. The dominating effect of A was also apparent in the AC combinations (FIG. 33b), when all tested sequences (A5C25 (SEQ ID NO: 25), A10C20 (SEQ ID NO: 26), A15C15 (SEQ ID NO: 27), A20C10 (SEQ ID NO: 28), A25C5 (SEQ ID NO: 29)) produced round nanoplates with rough surfaces, similar to that of poly A30.

However, for AG combinations (FIG. 33c), the nanoparticles synthesized in the presence of A5G25 (SEQ ID NO: 30) and A10G20 (SEQ ID NO: 31) resulted nanohexagons, similar to that of G20, indicating the effects from G dominated A. When the lengths of A and G were equal (A15G15, SEQ ID NO: 32), the edges of the nanohexagons showed roughness, apparently from equal competition from poly A and poly G segments. When the length of poly A segments increased to 20 (A20G10, SEQ ID NO: 33) or 25 (A25G5, SEQ ID NO: 34), the resulting nanoparticles were round nanoplates with rough surfaces, similar to that of A30, indicating that effects from A dominated G when the sequences contains more A's than G's. The results from AG combinations showed that the shaping effects of A and G compete with each other, and the one with longer length dominates.

For TC combinations (FIG. 33d), all tested DNA sequences (T5C25 (SEQ ID NO: 35), T10C20 (SEQ ID NO: 36), T15C15 (SEQ ID NO: 37), T20C10 (SEQ ID NO: 38), T25C5 (SEQ ID NO: 39)) produced flower-like nanoparticles with thin central areas and thick edges wrapped with multiple tips. With increasing poly T content, the edges grew thicker and the tips grew longer. In contrast to the TA, AC and AG combinations, the shapes produced from TC combinations were different from either poly T or poly C alone; instead the shaping effect from TC combinations was synergistic rather than competitive.

The TG combinations series was among the most interesting. As shown in FIG. 33e, the T5G25 (SEQ ID NO: 40) and T10G20 (SEQ ID NO: 41) produced nanohexagons similar to those formed in the presence of G20, indicating that the G shaping effect dominated. When the T segment and G segment were equal in length, the edges of the nanohexagons started to become thicker (see T15G15, SEQ ID NO: 42). Further increase in T segment length (e.g., T20G10 (SEQ ID NO: 43) and T25G5 (SEQ ID NO: 44)) produced six-pointed star, bowl-like nanoparticles, indicating that poly T's effects dominated when the sequence contained more T than G. The results showed that T and G were competing with one another, and the base present in greater amount determined the final shape.

The shaping effects of the CG combinations (FIG. 33f) shared some similarities with AG combinations (FIG. 33c), as the C10G20 (SEQ ID NO: 46) resulted in nanohexagons, where effects from G dominated. When the C segment was lengthened, round nanoplates with rough surfaces were formed (e.g., C25G5 (SEQ ID NO: 49), FIG. 33f).

The nucleotide effects on morphology and their interplay are summarized in FIG. 34 and Table 1. A competes with C or G, and the dominant nucleotide determines the nanoparticle morphology. C and G also compete with one another. C and T have a synergistic effect on one another, with the shape remaining substantially similar when both nucleotides are present regardless of the ratio of C:T. G and T have a competitive effect on one another. A dominates T, with A determining the morphology. Without wishing to be bound by any particular theory of operation, it is believed that A's dominance may arise from its higher binding affinity to gold surfaces compared to the binding affinity of T. This theory was investigated by placing the A nucleotides in the middle of the 30-mer, i.e., T10A10T10 (SEQ ID NO: 50). When poly T was present on both ends, nanoplates with thick edges and thin centers were formed (FIG. 35). Thus, T can control the shape if it is placed on both ends of the oligomer.

Based on the results and rules summarized in FIG. 34, nanoparticle morphologies were predicted based on particular di-deoxyribonucleotide combinations. The nanoparticles were then synthesized and evaluated. For example, based on the rules of FIG. 34, poly T20 by itself forms six-pointed nanostars, and the shaping effect of T segment is “smoothing, edge thickening”, while the shaping effect of G segment is “formation of a flat nanohexagon”. Because the interplay effect between the T segment and G segment is competitive, it was predicted that T20G5 (SEQ ID NO: 51) and T20G10 (SEQ ID NO: 43) should produce six-pointed star bowls, with the T effect dominating. As the G segment's length increased to 15 (T20G15, SEQ ID NO: 52), the shaping effect from G was predicted to become more evident, eventually forming nanohexagons. Synthesis of the nanoparticles demonstrated the accuracy of the predictions (FIG. 36a). T20G5 and T20G10 resulted in the formation of six-pointed star bowls, and T20G15 produced hexagons with thick edges. Further increasing the G segment length to 20 (T20G20, SEQ ID NO: 53) gave hexagons with flat surfaces. Thus, the particle morphology could be fine-tuned by gradually increasing the G segment's length.

In addition to predicting the nucleic acid sequence's influence on shape, surface roughness can be fine-tuned with the nucleobase composition. FIG. 34 suggests that an A segment has a “roughening” surface effect, while a G segment has a surface effect of “flattening,” and AG combinations have a “competitive” interplay effect. Based upon FIG. 34, it was predicted that the poly A segment should dominate in A20G5 (SEQ ID NO: 54) and A20G10 (SEQ ID NO: 33), thereby producing rough nanoplates. It also was predicted that increasing the G segment length would enhance its influence on the nanoparticle shape, producing flat hexagons. The results shown in FIG. 36b matched these predictions. The A20G5 indeed produced rough nanoplates, showing a shaping effect mainly from the poly A segment. When the poly G segment was increased to 10 or 15 bases, the surface roughness of the resulting nanoplates decreased. When the length of G segment was increased to 20 bases, nanoplates with smooth surfaces were produced. These results demonstrate that both the nanoparticle shape as well as the surface roughness can be fine-tuned by adjusting the oligomer sequence and length.

### Example 14

### Nanoparticle Synthesis with Combinations of Three Deoxyribonucleotides

To further evaluate DNA-encoded shape control of nanoparticles, more complex DNA containing three different types of deoxyribonucleotides was used to prepare gold nanoparticles from gold nanoprisms following the general procedure of Example 10.

Based on the principles in FIG. 34 and Table 1, it was hypothesized that nanoparticles synthesized from T20G10A10 (SEQ ID NO: 57) should display a combined shaping effect from its segments, namely T20 (flattening and edge thickening), G10 (forming flat hexagons), and A10 (surface roughening). This hypothesis was confirmed, as the T20G10A10 produced edge-decorated nanoplates, which have thin central areas and thick edges surrounded with small bumps (FIG. 37a).

As another example, the DNA sequence A10T20C10 (SEQ ID NO: 58) was designed to integrate the edge thickening effect from T20 with the surface roughening effects from A10 and C10. The combination was predicted to form nanoparticles with rough surfaces and thicker edges. As expected, rough donut-like nanoparticles were formed in high yield (FIG. 37b).

These results demonstrate that the shape of the resulted nanoparticles can be predicted by analyzing the shape control effects of each of their DNA components. Therefore nanoparticles with controlled morphologies can be rationally designed and synthesized with this DNA encoding method. One skilled in the art will understand that similar methods can be used with RNAs or PNAs.

### Example 15

### Diagnostic Imaging with Shaped Nanoparticles

Embodiments of the disclosed shaped nanoparticles (e.g., nanoflowers, nanohexagons, nanoplates, nanostars, nanopeanuts, etc.) may be used for diagnostic imaging, such as to visualize the location and/or size of a tumor. DNA-shaped gold nanoparticles can be synthesized as described in Examples 1 and 10. An antibody that recognizes an antigen on a tumor cell may be conjugated to the AuNPs by any suitable method. Tumor-specific antibodies are well known in the art. Alternatively, small molecules that specifically bind to tumor antigens can be used instead of antibodies. In one example, an aptamer specific for cancer cells is used.

Exemplary antibodies and small molecules that can be conjugated to the disclosed nanoparticles are provided in Table 5.

TABLE 5

| Tumor- | | |
|---|---|---|
| Specific | | Exemplary Antibody/Small |
| Antigen | Exemplary Tumors | Molecules |
| HER1 | adenocarcinoma | Cetuximab, panitumamab, |
| | | zalutumumab, nimotuzumab, |
| | | matuzumab. Small |
| | | molecule inhibitors gefitinib, |
| | | erlotinib, and lapatinib can |
| | | also be used. |
| HER2 | breast cancer, ovarian | Trastuzumab (Herceptin ®), |
| | cancer, stomach cancer, | pertuzumab |
| | uterine cancer | |
| CD25 | T-cell lymphoma | Daclizumab (Zenapax) |
| CEA | colorectal cancer, some | CEA-scan (Fab fragment, |
| | gastric cancers, biliary | approved by FDA), colo101 |
| | cancer | |
| Cancer | ovarian cancer, | OC125 monoclonal antibody |
| antigen 125 | mesothelioma, breast | |
| (CA125) | cancer | |
| Alpha- | hepatocellular carcinoma | ab75705 (available from |
| fetoprotein | | Abeam) and other |
| (AFP) | | commercially available AFP |
| | | antibodies |
| Lewis Y | colorectal cancer, biliary | B3 (Humanized) |
| | cancer | |
| TAG72 | adenocarcinomas including | B72.3 (FDA-approved |
| | colorectal, pancreatic, | monoclonal antibody) |
| | gastric, ovarian, | |
| | endometrial, mammary, | |
| | and non-small cell | |
| | lung cancer | |

The antibody (or small molecule) may be conjugated to the gold surface or to a DNA oligomer. The antibody-AuNP conjugates may then be administered to a subject using routine methods, for example by injection (for example intratumorally or i.v.). After waiting for a period of time sufficient to allow the conjugates to travel to and bind to the tumor cell antigens, the conjugates may be visualized by CT or x-ray imaging, thus permitting visualization of the tumor.

Alternatively, an antibody that recognizes a tumor cell antigen may be prepared. A second antibody that recognizes the anti-antigen antibody may be conjugated to the AuNPs. The anti-antigen antibody and the antibody-AuNP conjugates may be administered sequentially or simultaneously to the subject. After waiting for a period of time sufficient to allow the anti-antigen antibody to bind to the tumor cell antigen, and the antibody-AuNP conjugates to bind to the anti-antigen antibody, the conjugates may be visualized by CT or x-ray imaging.

In some examples, the antibody-AuNP conjugates are used to image tumor cells ex vivo. For example, tumor cells from a subject can be obtained (for example during a biopsy), and then incubated with the antibody-AuNP conjugates under conditions that permit the antibody to bind to its target protein. In some examples live cells are incubated with the antibody-AuNP conjugates, while in other examples killed or fixed cells are incubated with the antibody-AuNP conjugates. The cells can be processed for imaging (for example fixed and embedded), for example using electron microscopy.

### Example 16

### Photothermal Therapy with Shaped Nanoparticles

Embodiments of the disclosed shaped nanoparticles (e.g., nanoflowers, nanohexagons, nanoplates, nanostars, nanopeanuts, etc.) may be delivered to a target cell of interest for use in photothermal therapy. A DNA-shaped nanoparticle of a particular size and shape may be selected based on its absorbance of energy within a given wavelength range, e.g., near-infrared radiation. In certain embodiments, the shaped nanoparticle is conjugated to a moiety capable of recognizing and binding to the target cell. Suitable moieties include but are not limited to antibodies and fragments thereof, drug molecules, proteins, peptides, and aptamers.

In one example, AuNP conjugates may be delivered to tumor cells by the methods outlined in Example 15. Suitable AuNP doses may range from 20 mg per kg body weight to 20 g per kg body weight. Because AuNP conjugates are capable of absorbing near-infrared (NIR) radiation, the tumor site may be irradiated with NIR radiation (700 nm-1500 nm), such as from an NIR laser. For example, a red laser that emits light with a wavelength of 790 to 820 nm or 800 nm to 810 nm (such as 800 nm or 810 nm) may be used. In one example, the tumor is irradiated at a dose of at least 0.5 W/cm² for 2 to 60 minutes, for example 5 to 30 minutes or 3 to 10 minutes, such as at least 2 W/cm² for 2 to 60 minutes, for example 5 to 30 minutes or 3 to 10 minutes, at least 10 W/cm² for 2 to 60 minutes, for example 5 to 30 minutes or 3 to 10 minutes, or 0.5 to 50 W/cm² for 2 to 60 minutes, for example 5 to 30 minutes or 3 to 10 minutes. The tumor cells may be destroyed via photothermal heating caused when the AuNPs absorb energy from the laser.

### Example 17

### Drug Delivery with Shaped Nanoparticles

Embodiments of the disclosed shaped nanoparticles (e.g., nanoflowers, nanohexagons, nanoplates, nanostars, nanopeanuts, etc.) may be utilized to deliver a therapeutic drug molecule to a subject. For example, AuNPs can be synthesized as described in Examples 1 and 10. Therapeutic drug molecules may be conjugated to the AuNPs by any suitable means. The drug molecule may be conjugated to the gold surface or to a DNA oligomer. The drug-AuNP conjugate may then be administered to a subject as described above at a therapeutically effective dose. The drug-AuNP conjugates may be taken up by cells (e.g., by endocytosis or receptor-mediated endocytosis), thereby delivering drug to the cell interior. In one example, the drug is a chemotherapeutic agent, and is administered to a subject in order to treat a tumor in the subject.

Alternatively, the drug-AuNP conjugate may further be conjugated to an antibody that recognizes an antigen on a target cell. The drug-AuNP-antibody conjugate may be administered to a subject. The antibody may then bind to the target cell antigen, thereby delivering the drug to the immediate vicinity of the target cell while minimizing drug delivery to non-target cells.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

### REFERENCES

(1) Murray, C. B.; Sun, S. H.; Doyle, H.; Betley, T. Mrs Bulletin 2001, 26, 985-991.
(2) Jin, R. C.; Cao, Y. W.; Mirkin, C. A.; Kelly, K. L.; Schatz, G. C.; Zheng, J. G. Science 2001, 294, 1901-1903.
(3) Jana, N. R.; Gearheart, L.; Murphy, C. J. J. Phys. Chem. B 2001, 105, 4065-4067.
(4) Sun, Y. G.; Xia, Y. N. Science 2002, 298, 2176-2179.
(5) Daniel, M. C.; Astruc, D. Chem. Rev. 2004, 104, 293-346.
(6) Sonnichsen, C.; Reinhard, B. M.; Liphardt, J.; Alivisatos, A. P. Nat. Biotechnol. 2005, 23, 741-745.
(7) Huang, X. H.; El-Sayed, I. H.; Qian, W.; El-Sayed, M. A. J. Am. Chem. Soc. 2006, 128, 2115-2120.
(8) Tao, A. R.; Habas, S.; Yang, P. D. Small 2008, 4, 310-325.
(9) Xia, Y.; Xiong, Y.; Lim, B.; Skrabalak, S. E. Angew. Chem., Int. Ed. 2009, 48, 60-103.
(10) Seeman, N. C.; Nature 2003, 421, 427-431.
(11) Rothemund, P. W. K.; Nature 2006, 440, 297-302;
(12) Lu, J; Liu. J; Acc. Chem. Res. 2007, 40, 315-323.
(13) Wang, Z; Y. Lu, J. Mater. Chem. 2009, 19, 1788-1798.
(14) Braun, E.; Eichen, Y.; Sivan, U.; Ben-Yoseph, G. Nature 1998, 391, 775-778.
(15) Gu, Q.; Cheng, C. D.; Gonela, R.; Suryanarayanan, S.; Anabathula, S.; Dai, K.; Haynie, D. T. Nanotechnology 2006, 17, R14-R25.
(16) Alivisatos, A. P.; Johnsson, K. P.; Peng, X.; Wilson, T. E.; Loweth, C. J.; Bruchez Jr, M. P.; and Schultz, P. G; Nature 1996, 382, 609-611.
(17) Mirkin, C. A.; Letsinger, R. L.; Mucic, R. C.; Storhoff, J. J. Nature 1996, 382, 607-609.
(18) Warner, M. G.; Hutchison, J. E. Nat. Mater. 2003, 2, 272-277.
(19) Le, J. D.; Pinto, Y.; Seeman, N. C.; Musier-Forsyth, K.; Taton, T. A.; Kiehl, R. A. Nano Lett. 2004, 4, 2343-2347;
(20) Zhang, J.; Liu, Y.; Ke, Y.; Yan, H.; Nano Lett. 2006, 6, 248-251;
(21) Lee, J. H.; Wernette, D. P.; Yigit, M. V.; Liu, J.; Wang, Z; Lu, Y. Angew. Chem., Int. Ed. 2007, 46, 9006.
(22) Bigham, S. R.; Coffer, J. L. J. Phys. Chem. 1992, 96, 10581-10584.
(23) Ma, N.; Dooley, C. J.; Kelley, S. O. J. Am. Chem. Soc. 2006, 128, 12598-12599.
(24) Kumar, A.; Jakhmola, A. Langmuir 2007, 23, 2915-2918.
(25) Ma, N.; Yang, J.; Stewart, K. M.; Kelley, S. O. Langmuir 2007, 23, 12783-12787.
(26) Berti, L.; Burley, G. A. Nat. Nanotechnol. 2008, 3, 81-87.
(27) Wang, Q. B.; Liu, Y.; Ke, Y. G.; Yan, H. Angew. Chem., Int. Ed. 2008, 47, 316-319.
(28) Ma, N.; Sargent, E. H.; Kelley, S. O, Nat. Nanotechnol. 2009, 4, 121-125.
(29) Brown, S.; Sarikaya, M.; Johnson, E. J. Mol. Biol. 2000, 299, 725-735.
(30) Naik, R. R.; Stringer, S. J.; Agarwal, G.; Jones, S. E.; Stone, M. O, Nat. Mater. 2002, 1, 169-172.
(31) Banerjee, I. A.; Yu, L. T.; Matsui, H. Proc. Natl. Acad. Sci. U.S.A. 2003, 100, 14678-14682.
(32) Dickerson, M. B.; Sandhage, K. H.; Naik, R. R. Chem. Rev. 2008, 108, 4935-4978.
(33) Storhoff, J. J.; Elghanian, R.; Mirkin, C. A.; Letsinger, R. L. Langmuir 2002, 18, 6666-6670.
(34) Ostblom, M.; Liedberg, B.; Demers, L. M.; Mirkin, C. A. J. Phys. Chem. B 2005, 109, 15150-15160.
(35) Brown, K. R.; Natan, M. J. Langmuir 1998, 14, 726-728.
(36) Bakr, O. M.; Wunsch, B. H.; Stellacci, F. Chem. Mater. 2006, 18, 3297-3301.
(37) Poon, K.; Macgregor, R. B. Biopolymers 1998, 45, 427-434.
(38) Li, H. X.; Rothberg, L. J. J. Am. Chem. Soc. 2004, 126, 10958-10961.
(39) Li, H. X.; Rothberg, L. Proc. Natl. Acad. Sci. U.S.A. 2004, 101, 14036-14039.
(40) Zhao, L.; Ji, X.; Sun, X.; Li, J.; Yang, W.; Peng, X. J. Phys. Chem. C. 2009, 113, 16645-16651.
(41) Katz, E.; Willner, I. Angew. Chem., Int. Ed. 2004, 43, 6042-6108.
(42) Demers, L. M.; Mirkin, C. A.; Mucic, R. C.; Reynolds, R. A.; Letsinger, R. L.; Elghanian, R.; Viswanadham, G. Anal. Chem. 2000, 72, 5535-5541.
(43) Kannan, B.; Kulkarni, R. P.; Majumdar, A. Nano Lett. 2004, 4, 1521-1524.
(44) Nie, S.; Emory, S. R., Science 1997, 275, 1102-1106.
(45) Hao, E. C.; Bailey, R. C.; Schatz, G. G.; Hupp, J. T.; Li, S. Y. Nano Lett. 2004, 4, 327-330.
(46) Bakr, O. M.; Wunsch, B. H.; Stellacci, F. Chem. Mater. 2006, 18, 3297-3301.
(47) Wax, A.; Sokolov, K. Laser & Photon. Rev. 2009, 3, 146-158.
(48) Giljohann, D. A.; Seferos, D. S.; Patel, P. C.; Millstone, J. E.; Rosi, N. L.; Mirkin, C. A. Nano Lett. 2007, 7, 3818-3821.
(49) Chithrani, B. D.; Ghazani, A. A.; Chan, W. C. W. Nano Lett. 2006, 6, 662-668.
(50) Millstone, J. E.; Wei, W.; Jones, M. R.; Yoo, Y; Mirkin, C. A.; Nano. Lett. 2008, 8, 2526-2569.
(51) Newman, J. D. S.; Blanchard G. J. J. Nanoparticle Res. 2007, 9, 861-868.
(52) Liu Q.; et al., Mater. Sci. 2006, 41, 3657-3662.
(53) Popovtzer, R., et al., Nano. Lett. 2008, 8, 4593-4596.
(54) Hainfeld, J. et al., J. Pharmacy and Pharmacology 2008, 60, 977-985.
(55) Liu, J.; Lu, Y., Nat. Protoc. 2006, 1, 246.
(56) Demers, L. M., et al., Anal. Chem. 2000, 72, 5535.
(57) Farokhzad et al., Proc. Natl. Acad. Sci. U.S.A. 2006, 103, 6315.
(58) Giljohann et al., Nano Lett. 2007, 7, 3818.
(59) Hauck, T. S. et al., Small 2008, 4, 153.
(60) Jain et al., J. Phys. Chem. B 2006, 110, 7238.
(61) O'Neal et al., Cancer Letters 2004, 209, 171.
(62) Millstone et al., Nano Lett. August 2008, 8, 2526.
(63) Ostblom et al., J. Phys. Chem. B 2005, 109, 15150.

We claim:

1. A method for making a shaped nanoparticle, comprising:
providing a metal nanoseed;
selecting a nucleic acid oligomer having an oligomer sequence composition comprising at least two unique sequence segments, each sequence segment having a length of at least five nucleobases selected from the group consisting of A, C, G, T, U, and modified nucleobases;
adsorbing a plurality of the nucleic acid oligomers onto the metal nanoseed to produce an oligomer-functionalized nanoseed; and
depositing metal onto the oligomer-functionalized nanoseed to make a shaped nanoparticle, wherein the shaped nanoparticle has a morphology based at least in part on the oligomer sequence composition.

2. The method of claim 1, further comprising determining the morphology of the shaped nanoparticle, wherein the morphology comprises shape, surface characteristics, or a combination thereof.

3. The method of claim 2, further comprising:
providing a subsequent metal nanoseed having a morphology substantially similar to the metal nanoseed;
selecting a subsequent nucleic acid oligomer having a subsequent oligomer sequence composition comprising at least two subsequent sequence segments, each subsequent sequence segment comprising nucleobases selected from the group consisting of A, C, G, T, U, and modified nucleobases, wherein the subsequent oligomer sequence has a sequence variation in length of at least one sequence segment, type of nucleobases in at least one sequence segment, or both, compared to the oligomer sequence;
adsorbing a plurality of the subsequent nucleic acid oligomers onto the subsequent metal nanoseed to produce an oligomer-functionalized nanoseed;
depositing metal onto the oligomer-functionalized nanoseed to make a subsequent shaped nanoparticle, wherein the subsequent shaped nanoparticle has a morphology based at least in part on the subsequent oligomer sequence composition;
determining the morphology of the subsequent shaped nanoparticle, wherein the morphology comprises shape, surface characteristics, or a combination thereof; and
determining, based at least in part on the shaped nanoparticle's morphology and the subsequent shaped nanoparticle's morphology, a shape effect, a surface characteristic effect, or both, of the sequence variation.

4. The method of claim 3, further comprising determining a shape effect rule, a surface characteristic effect rule, or both, based at least in part on the oligomer sequence composition, the subsequent oligomer sequence composition, the shaped nanoparticle's morphology, and the subsequent shaped nanoparticle's morphology.

5. The method of claim 4, where determining the shape effect rule, the surface characteristic effect rule, or both, is further based at least in part on the nanoseed's morphology.

6. The method of claim 1, further comprising:
making a shaped nanoparticle having a desired morphology, comprising selecting a desired nanoparticle morphology before selecting the nucleic acid oligomer, wherein the desired nanoparticle morphology comprises a desired nanoparticle shape, desired nanoparticle surface characteristics, or a combination thereof, and
wherein selecting the nucleic acid oligomer comprises selecting an oligomer sequence composition based at least in part on a morphology of the metal nanoseed and the desired nanoparticle morphology.

7. The method of claim 6, where the oligomer sequence composition comprises at least two types of nucleobases selected from the group consisting of A, C, G, T, U, and modified nucleobases, and each sequence segment has a length of at least 5 nucleobases.

8. The method of claim 6, wherein:
the desired nanoparticle morphology comprises surface roughness, and selecting the oligomer sequence composition comprises selecting a sequence composition including at least one sequence segment comprising poly A, poly C, or a combination of A and C; or
the desired nanoparticle morphology comprises surface flattening, thickened edges relative to a center thickness of the shaped nanoparticle, or a combination thereof, and selecting the oligomer sequence composition comprises selecting a sequence composition including at least one sequence segment comprising poly T.

9. The method of claim 8, where increasing the percent poly T in the nucleic acid oligomer increases an edge thickness of the shaped nanoparticle relative to the center thickness of the shaped nanoparticle.

10. The method of claim 6, where the desired nanoparticle morphology comprises a hexagonal shape, and selecting the oligomer sequence composition comprises selecting a sequence composition including at least 50% poly G.

11. The method of claim 10, where the desired nanoparticle morphology further comprises a flattened surface, thickened edges relative to a center thickness of the shaped nanoparticle, or a combination thereof, and selecting the oligomer sequence composition further comprises selecting a sequence composition including at least one sequence segment comprising poly T, wherein increasing the percent poly T in the nucleic acid oligomer increases the edge thickness relative to the center thickness.

12. The method of claim 10, where the desired nanoparticle morphology further comprises a rough surface, and selecting the oligomer sequence composition further comprises selecting a sequence composition including at least one sequence segment comprising poly A, poly C, or a combination thereof.

13. The method of claim 6, where the desired nanoparticle morphology comprises a six-pointed bowl shape, and selecting the oligomer sequence composition comprises selecting a sequence composition including at least one sequence segment comprising poly T and at least one sequence segment comprising poly G, wherein the nucleic acid oligomer is at least 50% poly T, and wherein increasing the percent poly T in the nucleic acid oligomer increases an edge thickness of the shaped nanoparticle relative to a center thickness of the shaped nanoparticle.

14. The method of claim 6, where the desired nanoparticle morphology comprises a flower shape with multiple edge tips having an average length and an average thickness, and selecting the oligomer sequence composition comprises selecting a sequence composition including at least one sequence segment comprising poly T and at least one sequence segment comprising poly C, wherein increasing the percent poly T in the nucleic acid oligomer increases the average length and the average thickness of the edge tips.

15. A shaped nanoparticle made by the method of claim 1.

16. A method of delivering a shaped nanoparticle to a target cell, comprising:
providing a shaped nanoparticle made by the method of claim 1, and
contacting the shaped nanoparticle with the target cell under conditions that allow the shaped nanoparticle to bind to and/or enter the cell, thereby delivering the shaped nanoparticle to the target cell.

17. The method of claim 16 where providing the shaped nanoparticle comprises providing a conjugate comprising the shaped nanoparticle made by the method of claim 1 and a molecule of interest conjugated to the shaped nanoparticle, wherein the molecule of interest is an antibody, a drug molecule, a protein, a peptide, an aptamer, or a nucleic acid molecule, the method further comprising:
contacting the conjugate with the cell under conditions that allow the conjugate to bind to and/or enter the cell, thereby delivering the shaped nanoparticle and the molecule of interest to the cell.

18. The method of claim 17, where the molecule of interest is an antibody that is capable of recognizing and binding to a target antigen on the cell's surface.

19. The method of claim 18, where the conjugate further comprises a subsequent molecule of interest conjugated to the shaped nanoparticle, wherein the subsequent molecule of interest is a drug molecule, a protein, a peptide, an aptamer, or a nucleic acid molecule.

20. A method, comprising:
administering to a subject a shaped nanoparticle comprising a metal nanoparticle, and a plurality of nucleic acid oligomers, each nucleic acid oligomer having an oligomer sequence composition comprising at least two unique sequence segments, each sequence segment having a length of at least five nucleobases selected from the group consisting of A, C, G, T, U, and modified nucleobases; and
detecting the shaped nanoparticle in the subject.


SEQUENCE LISTING (61 sequences)

SEQ ID NO: 1 - DNA, 73 bp, Artificial Sequence
  Synthetic oligonucleotide
ttggtagtagtgattgtaatggtagtgatttttttttttttttcccccccccccccccccccccccccccccc

SEQ ID NO: 2 - DNA, 73 bp, Artificial Sequence
  Synthetic oligonucleotide
ttggtggtggtggttgtggtggtggtggtttttttttttttttcccccccccccccccccccccccccccccc

SEQ ID NO: 3 - DNA, 30 bp, Artificial Sequence
  Synthetic oligonucleotide
agtcacgtatacagctcatgatcagtcagt

SEQ ID NO: 4 - DNA, 30 bp, Artificial Sequence
  Synthetic oligonucleotide
aaaaaaaaaaaaaaaaaaaaaaaaaaaaaa

SEQ ID NO: 5 - DNA, 30 bp, Artificial Sequence
  Synthetic oligonucleotide
tttttttttttttttttttttttttttttt

SEQ ID NO: 6 - DNA, 30 bp, Artificial Sequence
  Synthetic oligonucleotide
cccccccccccccccccccccccccccccc

SEQ ID NO: 7 - DNA, 20 bp, Artificial Sequence
  Synthetic oligonucleotide
gggggggggggggggggggg

SEQ ID NO: 8 - DNA, 5 bp, Artificial Sequence
  Synthetic oligonucleotide
aaaaa

SEQ ID NO: 9 - DNA, 10 bp, Artificial Sequence
  Synthetic oligonucleotide
aaaaaaaaaa

SEQ ID NO: 10 - DNA, 20 bp, Artificial Sequence
  Synthetic oligonucleotide
aaaaaaaaaaaaaaaaaaaa

SEQ ID NO: 11 - DNA, 5 bp, Artificial Sequence
  Synthetic oligonucleotide
ttttt

SEQ ID NO: 12 - DNA, 10 bp, Artificial Sequence
  Synthetic oligonucleotide
tttttttttt

SEQ ID NO: 13 - DNA, 20 bp, Artificial Sequence
  Synthetic oligonucleotide
tttttttttttttttttttt

SEQ ID NO: 14 - DNA, 5 bp, Artificial Sequence
  Synthetic oligonucleotide
ccccc

SEQ ID NO: 15 - DNA, 10 bp, Artificial Sequence
  Synthetic oligonucleotide
cccccccccc

SEQ ID NO: 16 - DNA, 20 bp, Artificial Sequence
  Synthetic oligonucleotide
cccccccccccccccccccc

SEQ ID NO: 17 - DNA, 5 bp, Artificial Sequence
  Synthetic oligonucleotide
ggggg

SEQ ID NO: 18 - DNA, 10 bp, Artificial Sequence
  Synthetic oligonucleotide
gggggggggg

SEQ ID NO: 19 - DNA, 15 bp, Artificial Sequence
  Synthetic oligonucleotide
ggggggggggggggg

SEQ ID NO: 20 - DNA, 30 bp, Artificial Sequence
  Synthetic oligonucleotide
tttttaaaaaaaaaaaaaaaaaaaaaaaaa

SEQ ID NO: 21 - DNA, 30 bp, Artificial Sequence
  Synthetic oligonucleotide
ttttttttttaaaaaaaaaaaaaaaaaaaa

SEQ ID NO: 22 - DNA, 30 bp, Artificial Sequence
  Synthetic oligonucleotide
tttttttttttttttaaaaaaaaaaaaaaa

SEQ ID NO: 23 - DNA, 30 bp, Artificial Sequence
  Synthetic oligonucleotide
ttttttttttttttttttttaaaaaaaaaa

SEQ ID NO: 24 - DNA, 30 bp, Artificial Sequence
  Synthetic oligonucleotide
tttttttttttttttttttttttttaaaaa

SEQ ID NO: 25 - DNA, 30 bp, Artificial Sequence
  Synthetic oligonucleotide
aaaaaccccccccccccccccccccccccc

SEQ ID NO: 26 - DNA, 30 bp, Artificial Sequence
  Synthetic oligonucleotide
aaaaaaaaaacccccccccccccccccccc

SEQ ID NO: 27 - DNA, 30 bp, Artificial Sequence
  Synthetic oligonucleotide
aaaaaaaaaaaaaaaccccccccccccccc

SEQ ID NO: 28 - DNA, 30 bp, Artificial Sequence
  Synthetic oligonucleotide
aaaaaaaaaaaaaaaaaaaacccccccccc

SEQ ID NO: 29 - DNA, 30 bp, Artificial Sequence
  Synthetic oligonucleotide
aaaaaaaaaaaaaaaaaaaaaaaaaccccc

SEQ ID NO: 30 - DNA, 30 bp, Artificial Sequence
  Synthetic oligonucleotide
aaaaaggggggggggggggggggggggggg

SEQ ID NO: 31 - DNA, 30 bp, Artificial Sequence
  Synthetic oligonucleotide
aaaaaaaaaagggggggggggggggggggg

SEQ ID NO: 32 - DNA, 30 bp, Artificial Sequence
  Synthetic oligonucleotide
aaaaaaaaaaaaaaaggggggggggggggg

SEQ ID NO: 33 - DNA, 30 bp, Artificial Sequence
  Synthetic oligonucleotide
aaaaaaaaaaaaaaaaaaaagggggggggg

SEQ ID NO: 34 - DNA, 30 bp, Artificial Sequence
  Synthetic oligonucleotide
aaaaaaaaaaaaaaaaaaaaaaaaaggggg

SEQ ID NO: 35 - DNA, 30 bp, Artificial Sequence
  Synthetic oligonucleotide
tttttccccccccccccccccccccccccc

SEQ ID NO: 36 - DNA, 30 bp, Artificial Sequence
  Synthetic oligonucleotide
ttttttttttcccccccccccccccccccc

SEQ ID NO: 37 - DNA, 30 bp, Artificial Sequence
  Synthetic oligonucleotide
tttttttttttttttccccccccccccccc

SEQ ID NO: 38 - DNA, 30 bp, Artificial Sequence
  Synthetic oligonucleotide
ttttttttttttttttttttcccccccccc

SEQ ID NO: 39 - DNA, 30 bp, Artificial Sequence
  Synthetic oligonucleotide
tttttttttttttttttttttttttccccc

SEQ ID NO: 40 - DNA, 30 bp, Artificial Sequence
  Synthetic oligonucleotide
tttttggggggggggggggggggggggggg

SEQ ID NO: 41 - DNA, 30 bp, Artificial Sequence
  Synthetic oligonucleotide
ttttttttttgggggggggggggggggggg

SEQ ID NO: 42 - DNA, 30 bp, Artificial Sequence
  Synthetic oligonucleotide
tttttttttttttttggggggggggggggg

SEQ ID NO: 43 - DNA, 30 bp, Artificial Sequence
  Synthetic oligonucleotide
ttttttttttttttttttttgggggggggg

SEQ ID NO: 44 - DNA, 30 bp, Artificial Sequence
  Synthetic oligonucleotide
tttttttttttttttttttttttttggggg

SEQ ID NO: 45 - DNA, 30 bp, Artificial Sequence
  Synthetic oligonucleotide
cccccggggggggggggggggggggggggg

SEQ ID NO: 46 - DNA, 30 bp, Artificial Sequence
  Synthetic oligonucleotide
ccccccccccgggggggggggggggggggg

SEQ ID NO: 47 - DNA, 30 bp, Artificial Sequence
  Synthetic oligonucleotide
cccccccccccccccggggggggggggggg

SEQ ID NO: 48 - DNA, 30 bp, Artificial Sequence
  Synthetic oligonucleotide
ccccccccccccccccccccgggggggggg

SEQ ID NO: 49 - DNA, 30 bp, Artificial Sequence
  Synthetic oligonucleotide
cccccccccccccccccccccccccggggg

SEQ ID NO: 50 - DNA, 30 bp, Artificial Sequence
  Synthetic oligonucleotide
ttttttttttaaaaaaaaaatttttttttt

SEQ ID NO: 51 - DNA, 25 bp, Artificial Sequence
  Synthetic oligonucleotide
ttttttttttttttttttttggggg

SEQ ID NO: 52 - DNA, 35 bp, Artificial Sequence
  Synthetic oligonucleotide
ttttttttttttttttttttggggggggggggggg

SEQ ID NO: 53 - DNA, 40 bp, Artificial Sequence
  Synthetic oligonucleotide
ttttttttttttttttttttgggggggggggggggggggg

SEQ ID NO: 54 - DNA, 25 bp, Artificial Sequence
  Synthetic oligonucleotide
aaaaaaaaaaaaaaaaaaaaggggg

SEQ ID NO: 55 - DNA, 35 bp, Artificial Sequence
  Synthetic oligonucleotide
aaaaaaaaaaaaaaaaaaaaggggggggggggggg

SEQ ID NO: 56 - DNA, 40 bp, Artificial Sequence
  Synthetic oligonucleotide
aaaaaaaaaaaaaaaaaaaagggggggggggggggggggg

SEQ ID NO: 57 - DNA, 40 bp, Artificial Sequence
  Synthetic oligonucleotide
ttttttttttttttttttttggggggggggaaaaaaaaaa

SEQ ID NO: 58 - DNA, 40 bp, Artificial Sequence
  Synthetic oligonucleotide
aaaaaaaaaattttttttttttttttttttcccccccccc

SEQ ID NO: 59 - DNA, 24 bp, Artificial Sequence
  Synthetic oligonucleotide
gcagttgatcctttggataccctg

SEQ ID NO: 60 - DNA, 63 bp, Artificial Sequence
  Synthetic oligonucleotide
acagcatccccatgtgaacaatcgcattgtgattgttacggtttccgcctcatggacgtgctg

SEQ ID NO: 61 - DNA, 48 bp, Artificial Sequence
  Synthetic oligonucleotide
taactgctgcgccgccgggaaaatactgtacggttagatttttttttt